(19) United States
(12) Patent Application Publication
(10) Pub. No.: US 2024/0279572 A1
(43) Pub. Date: Aug. 22, 2024

(54) Polypeptides Having Alpha-Mannan Degrading Activity And Polynucleotides Encoding Same

(71) Applicant: Novozymes A/S, Bagsvaerd (DK)

(72) Inventors: Jesper Salomon, Holte (DK); Klaus Gori, Dyssegaard (DK); Lorena González Palmén, Åkarp (SE); Kenneth Jensen, Oelsted (DK); Nikolaj Spodsberg, Holte (DK); Mary Ann Stringer, Søborg (DK); Kristian Bertel Roemer M. Krogh, Bagsvaerd (DK); Morten Gjermansen, Greve (DK)

(73) Assignee: Novozymes A/S, Bagsvaerd (DK)

(21) Appl. No.: 18/639,702

(22) Filed: Apr. 18, 2024

Related U.S. Application Data
(62) Division of application No. 17/280,798, filed on Mar. 26, 2021, now Pat. No. 11,993,762.

(30) Foreign Application Priority Data
Oct. 3, 2018 (EP) 18198448.5

(51) Int. Cl. C11D 3/386; C12N 9/24; C12N 15/09; C12N 15/11; C12P 21/00; C12P 21/02

(52) U.S. Cl. CPC C11D 3/38636; C12N 9/2402; C12Y 302/01078; C12N 15/09; C12N 15/11; C12P 21/00; C12P 21/02

(57) ABSTRACT

The invention relates to compositions comprising and uses of polypeptides having alpha-mannan degrading activity. The present invention relates to polypeptides having alpha-mannan degrading activity, and polynucleotides encoding the polypeptides. The invention also relates to nucleic acid constructs, vectors, and host cells comprising the polynucleotides as well as methods of producing and using the polypeptides.

### REFERENCE TO A SEQUENCE LISTING

This application contains a Sequence Listing in computer readable form, which is incorporated herein by reference.

### BACKGROUND OF THE INVENTION

### Field of the Invention

The invention relates to compositions comprising and uses of polypeptides having alpha-mannan degrading activity. The present invention relates to polypeptides having alpha-mannan degrading activity, and polynucleotides encoding the polypeptides. The invention also relates to nucleic acid constructs, vectors, and host cells comprising the polynucleotides as well as methods of producing and using the polypeptides.

### Description of the Related Art

Enzymes have been used in detergents for decades. Usually a cocktail of various enzymes is added to detergent compositions. The enzyme cocktail often comprises various enzymes, wherein each enzyme targets it specific substrate, e.g., amylases are active towards starch stains, proteases towards protein stains, and so forth.

Mannans are known in the art as plant polysaccharides with a backbone of β-1,4-linked D-mannopyranosyl residues, which can contain galactose or acetyl substitutions and may have glucose residues in the backbone. The main enzyme type participating in the degradation of mannans are endo-1,4-β-mannanases (EC 3.2.1.78), which hydrolyze the internal glycoside bonds in the mannan backbone. Beta-mannanases, which are often simply referred to in the literature as mannanases, have been used for many years in the detergent industry for effective stain removal of plant-based stains.

Another important type of mannan is the cell wall polysaccharide found, for example, in yeast cell walls, and known as alpha-mannan. In contrast to the plant-based beta-mannan polysaccharides noted above, these alpha-mannans contain mannose residues connected through an alpha-(1,6)-linked linear backbone, with alpha-(1,2) and alpha-(1,3) linked branches, which may also be phosphorylated.

Alpha-mannans are also components of biofilm. Textiles surface and hard surfaces, such as dishes or the inner space of a laundry machine enduring a number of wash cycles, become soiled with many different types of soiling which may compose of proteins, grease, starch etc. One type of soiling may be organic matter, such as biofilm, extracellular polymeric substance (EPS), etc. Organic matter composes different molecules such as polysaccharides, extracellular DNA (eDNA), and proteins. Some organic matter composes an extracellular polymeric matrix, which may be sticky or glueing, which when present on textile, attracts soils and may course redeposition or backstaining of soil resulting in a greying of the textile. Additionally, organic matters such as biofilms often cause malodor issue as various malodor molecules can be adhered by the polysaccharides, extracellular DNA (eDNA), and proteins in the complex extracellular matrix and be slowly released out to cause consumer noticeable malodor issue.

Thus, it could be advantageous to use enzymes in applications where alpha-mannan needs to be degraded.

According to the online carbohydrate-active enzyme (“CAZy”) database (available at cazy.org), alpha-mannan degrading enzymes have been found in glycoside hydrolase families including 76, 92, and 99. The present invention provides polypeptides having alpha-mannanase activity and polynucleotides encoding the polypeptides that are highly active in degrading alpha-mannan, and therefore could be used in the aforementioned applications.

### SUMMARY OF THE INVENTION

The invention relates to cleaning compositions comprising at least one polypeptide having alpha-mannan degrading activity or a variant or a fragment thereof having alpha-mannan degrading activity; and a cleaning composition component.

The present invention provides isolated or purified polypeptides having alpha-mannan degrading activity and polynucleotides encoding the polypeptides. In an embodiment, the polypeptide having alpha-mannan degrading activity belongs to glycosyl hydrolase family 76 (GH76), glycosyl hydrolase family 92 (GH92), glycosyl hydrolase family 99 (GH99), in particular to glycosyl hydrolase family 76.

One aspect relates to polypeptides comprising one or more of the motifs [YND]DD[QINLEM] (SEQ ID NO: 60) and GG[ILMV]X[WS] (SEQ ID NO: 61).

One aspect relates to polypeptides comprises the motif [RK][NLT]XXX[NTV]XP[GTLYISAVFNM] (SEQ ID NO: 64).

One aspect relates to polypeptides of the KNTPA clade and of bacterial origin.

One aspect relates to polypeptides comprising the motif GA]XX[AVL][ML]X[MA][ATV][EATV] (SEQ ID NO: 62) or the motif LA[EQ]X[VL][YF] (SEQ ID NO: 63).

One aspect relates to polypeptides of the AMXAAE clade and of fungal origin.

One aspect relates to polypeptides comprising the motif N[EQD][WFY][HG]E (SEQ ID NO: 66).

Accordingly, the present invention relates to isolated or purified polypeptides having alpha-mannan degrading activity selected from the group consisting of: a polypeptide having at least 60% sequence identity to the polypeptide of SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 54, or SEQ ID NO: 57 or a fragment thereof having alpha-mannan degrading activity.

Accordingly, the present invention relates to isolated or purified polypeptides having alpha-mannan degrading activity selected from the group consisting of:
(a) a polypeptide having at least 60% sequence identity to the mature polypeptide of SEQ ID NO: 2, SEQ ID NO: 5, SEQ ID NO: 8, SEQ ID NO: 11, SEQ ID NO: 14, SEQ ID NO: 17, SEQ ID NO: 20, SEQ ID NO: 23, SEQ ID NO: 26, SEQ ID NO: 29, SEQ ID NO: 32, SEQ ID NO: 35, SEQ ID NO: 38, SEQ ID NO: 41, SEQ ID NO: 44, SEQ ID NO: 47, SEQ ID NO: 53, SEQ ID NO: 56;
(b) a polypeptide encoded by a polynucleotide that hybridizes under medium stringency conditions with the full-length complement of the mature polypeptide coding sequence of SEQ ID NO: 1, SEQ ID NO: 4, SEQ ID NO: 7, SEQ ID NO: 10, SEQ ID NO: 13, SEQ ID NO: 16, SEQ ID NO: 19, SEQ ID NO: 22, SEQ ID NO: 25, SEQ ID NO: 28, SEQ ID NO: 31 or the cDNA sequence thereof, SEQ ID NO: 34 or the cDNA sequence thereof, SEQ ID NO: 37 or the cDNA sequence thereof, SEQ ID NO: 40 or the cDNA sequence thereof, SEQ ID NO: 43 or the cDNA sequence thereof, SEQ ID NO: 46 or the cDNA sequence thereof, SEQ ID NO: 49 or the cDNA sequence thereof, SEQ ID NO: 52 or the cDNA sequence thereof, SEQ ID NO: 55;
(c) a polypeptide encoded by a polynucleotide having at least 60% sequence identity to the mature polypeptide coding sequence of SEQ ID NO: 1, SEQ ID NO: 4, SEQ ID NO: 7, SEQ ID NO: 10, SEQ ID NO: 13, SEQ ID NO: 16, SEQ ID NO: 19, SEQ ID NO: 22, SEQ ID NO: 25, SEQ ID NO: 28, SEQ ID NO: 31 or the cDNA sequence thereof, SEQ ID NO: 34 or the cDNA sequence thereof, SEQ ID NO: 37 or the cDNA sequence thereof, SEQ ID NO: 40 or the cDNA sequence thereof, SEQ ID NO: 43 or the cDNA sequence thereof, SEQ ID NO: 46 or the cDNA sequence thereof, SEQ ID NO: 46 or the cDNA sequence thereof, SEQ ID NO: 49 or the cDNA sequence thereof, SEQ ID NO: 52 or the cDNA sequence thereof, SEQ ID NO: 55;
(d) a fragment of the polypeptide of (a), (b), or (c), that has alpha-mannan degrading activity.

The present invention also relates to isolated or purified polynucleotides encoding the polypeptides of the present invention; nucleic acid constructs; recombinant expression vectors; recombinant host cells comprising the polynucleotides; and methods of producing the polypeptides.

The present invention further relates to granules comprising a core particle and one or more coatings, wherein the granule comprises a polypeptide having alpha-mannan degrading activity as defined above, and liquid compositions comprising a polyol and a polypeptide having alpha-mannan degrading activity, wherein the polypeptide is as defined above.

The invention further relates to uses of the peptide in various applications such as degrading alpha-mannan, laundering, washing, cleaning, feed, food, extracting coffee, degrading cellulosic material, producing a fermentation product, isolated polynucleotides encoding the polypeptides of the invention, recombinant host cells and method of producing the polypeptide of the invention.

### Overview of Sequences

SEQ ID NO: 1 DNA encoding full length polypeptide from Paenibacillus glycanilyticus
SEQ ID NO: 2 polypeptide derived from SEQ ID NO: 1
SEQ ID NO: 3 mature polypeptide obtained from Paenibacillus glycanilyticus
SEQ ID NO: 4 DNA encoding full length polypeptide from Bacillus acidicola
SEQ ID NO: 5 polypeptide derived from SEQ ID NO: 4
SEQ ID NO: 6 mature polypeptide obtained from Bacillus acidicola
SEQ ID NO: 7 DNA encoding full length polypeptide from Bacillus sp.
SEQ ID NO: 8 polypeptide derived from SEQ ID NO: 7
SEQ ID NO: 9 mature polypeptide obtained from Bacillus sp.
SEQ ID NO: 10 DNA encoding full length polypeptide from Paenibacillus sp. A
SEQ ID NO: 11 polypeptide derived from SEQ ID NO: 10
SEQ ID NO: 12 mature polypeptide obtained from Paenibacillus sp. A
SEQ ID NO: 13 DNA encoding full length polypeptide from Paenibacillus sp. B
SEQ ID NO: 14 polypeptide derived from SEQ ID NO: 13
SEQ ID NO: 15 mature polypeptide obtained from Paenibacillus sp. B
SEQ ID NO: 16 DNA encoding full length polypeptide from Paenibacillus sp. C
SEQ ID NO: 17 polypeptide derived from SEQ ID NO: 16
SEQ ID NO: 18 mature polypeptide obtained from Paenibacillus sp. C
SEQ ID NO: 19 DNA encoding full length polypeptide from Microbial community B
SEQ ID NO: 20 polypeptide derived from SEQ ID NO: 19
SEQ ID NO: 21 mature polypeptide obtained from Microbial community B
SEQ ID NO: 22 DNA encoding full length polypeptide from Microbial community H
SEQ ID NO: 23 polypeptide derived from SEQ ID NO: 22
SEQ ID NO: 24 mature polypeptide obtained from Microbial community H
SEQ ID NO: 25 DNA encoding full length polypeptide from Bacillus novalis
SEQ ID NO: 26 polypeptide derived from SEQ ID NO: 25
SEQ ID NO: 27 mature polypeptide obtained from Bacillus novalis
SEQ ID NO: 28 DNA encoding full length polypeptide from Chryseobacterium sp.
SEQ ID NO: 29 polypeptide derived from SEQ ID NO: 28
SEQ ID NO: 30 mature polypeptide obtained from Chryseobacterium sp.
SEQ ID NO: 31 DNA encoding full length polypeptide from Aspergillus aculeatus
SEQ ID NO: 32 polypeptide derived from SEQ ID NO: 31
SEQ ID NO: 33 mature polypeptide obtained from Aspergillus aculeatus
SEQ ID NO: 34 DNA encoding full length polypeptide from Aspergillus aculeatus
SEQ ID NO: 35 polypeptide derived from SEQ ID NO: 34
SEQ ID NO: 36 mature polypeptide obtained from Aspergillus aculeatus
SEQ ID NO: 37 DNA encoding full length polypeptide from Aspergillus aculeatus
SEQ ID NO: 38 polypeptide derived from SEQ ID NO: 37
SEQ ID NO: 39 mature polypeptide obtained from Aspergillus aculeatus
SEQ ID NO: 40 DNA encoding full length polypeptide from Aspergillus aculeatus
SEQ ID NO: 41 polypeptide derived from SEQ ID NO: 40
SEQ ID NO: 42 mature polypeptide obtained from Aspergillus aculeatus
SEQ ID NO: 43 DNA encoding full length polypeptide from Humicola insolens
SEQ ID NO: 44 polypeptide derived from SEQ ID NO: 43
SEQ ID NO: 45 mature polypeptide obtained from Humicola insolens
SEQ ID NO: 46 DNA encoding full length polypeptide from Humicola insolens
SEQ ID NO: 47 polypeptide derived from SEQ ID NO: 46
SEQ ID NO: 48 mature polypeptide obtained from Humicola insolens
SEQ ID NO: 49 DNA encoding full length polypeptide from Humicola insolens
SEQ ID NO: 50 polypeptide derived from SEQ ID NO: 43
SEQ ID NO: 51 mature polypeptide obtained from Humicola insolens
SEQ ID NO: 52 DNA encoding full length polypeptide from Humicola insolens
SEQ ID NO: 53 polypeptide derived from SEQ ID NO: 43
SEQ ID NO: 54 mature polypeptide obtained from Humicola insolens
SEQ ID NO: 55 DNA encoding truncated polypeptide from Microbial community H
SEQ ID NO: 56 polypeptide derived from SEQ ID NO: 55
SEQ ID NO: 57 mature polypeptide obtained from Microbial community H
SEQ ID NO: 58 is a secretion signal
SEQ ID NO: 59 is a His tag
SEQ ID NO: 60 is a motif [YND]DD[QINLEM]
SEQ ID NO: 61 is a motif GG[ILMV]X[WS]
SEQ ID NO: 62 is a motif [GA]XX[AVL][ML]X[MA][ATV][EATV]
SEQ ID NO: 63 is a motif LA[EQ]X[VL][YF]
SEQ ID NO: 64 is a motif [RK][NLT]XXX[NTV]XP[GTLYISAVFNM]
SEQ ID NO: 65 is a motif KNTPANAPA
SEQ ID NO: 66 is a motif N[EQD][WFY][HG]E
SEQ ID NO: 67 is a beta-mannanase obtainable from Bacillus bogoriensis
SEQ ID NO: 68 is a beta-mannanase obtainable from Paenibacillus sp. (PspMan4)
SEQ ID NO: 69 is a beta-mannanase obtainable from Bacillus hemicellulosilyticus

### Definitions

In accordance with this detailed description, the following definitions apply. Note that the singular forms “a,” “an,” and “the” include plural references unless the context clearly dictates otherwise.

Reference to “about” a value or parameter herein includes aspects that are directed to that value or parameter per se. For example, description referring to “about X” includes the aspect “X”.

Unless defined otherwise or clearly indicated by context, all technical and scientific terms used herein have the same meaning as commonly understood by one of ordinary skill in the art to which this invention belongs.

Alpha-Mannan Degrading Enzyme: The term “alpha-mannan degrading enzyme” or “polypeptide having alpha-mannan degrading activity” means an enzyme having hydrolase activity on alpha-mannan. Relevant are enzymes having alpha-mannanase and/or alpha-mannosidase activity. In particular, the polypeptide having alpha-mannan degrading activity includes glycoside hydrolase domains GH76, GH92, or GH99, as defined in CAZY (available at cazy.org, and as described in Lombard V, et al. 2014, Nucleic Acids Res 42:D490-D495). These can include enzyme activities such as alpha-1,6-mannanase (EC 3.2.1.101), alpha-1,2-mannase; mannosyl-oligosaccharide alpha-1,2-mannosidase (EC 3.2.1.113); mannosyl-oligosaccharide alpha-1,3-mannosidase (EC 3.2.1.-); mannosyl-oligosaccharide alpha-1,6-mannosidase (EC 3.2.1.-); alpha-mannosidase (EC 3.2.1.24); alpha-1,2-mannosidase (EC 3.2.1.-); alpha-1,3-mannosidase (EC 3.2.1.-); alpha-1,4-mannosidase (EC 3.2.1.-); mannosyl-1-phosphodiester alpha-1,P-mannosidase (EC 3.2.1.-); glycoprotein endo-alpha-1,2-mannosidase (EC 3.2.1.130); and/or mannan endo-1,2-alpha-mannanase (3.2.1.-) activities.

Beta-Mannanase: The term “beta-mannanase” or “galactomannase” as used herein refers to a beta-mannanase enzyme defined as the officially named mannan endo-1,4-beta-mannosidase and having the alternative names beta-mannanase and endo-1,4-mannase. The beta-mannanase term also means a polypeptide or polypeptide domain of an enzymes that has the ability to catalyze the cleavage or hydrolysis of (1->4) beta-D-mannosidic linkages of mannans, galactomannans, glucomannans, and galactoglucmannans. Thus, it means that the beta-mannanase has beta-mannanase activity (EC 3.2.1.78). For purposes of the present invention, beta-mannanase activity is determined according to the procedure described in the Examples. In one aspect, the variants of the present invention have at least 20%, e.g., at least 40%, at least 50%, at least 60%, at least 70%, at least 80%, at least 90%, at least 95%, or at least 100% of the mannanase activity of the polypeptide of SEQ ID NO: 67.

Binding module: The term “binding module” means the region of an enzyme that mediates binding of the enzyme to amorphous regions of a cellulose substrate. The carbohydrate binding module (CBD) is typically found either at the N-terminal or at the C-terminal extremity of an alpha-mannan degrading enzyme.

Catalytic domain: The term “catalytic domain” means the region of an enzyme containing the catalytic machinery of the enzyme.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Fragment: The term “fragment” means a polypeptide, a catalytic domain, or a carbohydrate binding module having one or more (e.g., several) amino acids absent from the amino and/or carboxyl terminus of a mature polypeptide or domain; wherein the fragment has alpha-mannan degrading activity. In some embodiments, a fragment contains at least 90% of the length of the mature polypeptide, such as at least 317 amino acid residues of SEQ ID NO: 2 or SEQ ID NO: 3, at least 314 amino acid residues of SEQ ID NO: 5 or SEQ ID NO: 6, at least 315 amino acid residues of SEQ ID NO: 8 or SEQ ID NO: 9, at least 431 amino acids of SEQ ID NO: 11 or SEQ ID NO: 12, at least 440 amino acids of SEQ ID NO: 14 or SEQ ID NO: 15, at least 313 amino acids of SEQ ID NO: 17 or SEQ ID NO: 18, at least 484 amino acids of SEQ ID NO: 20 or SEQ ID NO: 21, at least 487 amino acids of SEQ ID NO: 23 or SEQ ID NO: 24, at least 1240 amino acids of SEQ ID NO: 26 or SEQ ID NO: 27, at least 307 amino acids of SEQ ID NO: 29 or SEQ ID NO: 30, at least 332 amino acids of SEQ ID NO: 32 or SEQ ID NO: 33, at least 388 amino acids of SEQ ID NO: 35 or SEQ ID NO: 36, at least 345 amino acids of SEQ ID NO: 38 or SEQ ID NO: 39, at least 342 amino acids of SEQ ID NO: 41 or SEQ ID NO: 42, at least 352 amino acids of SEQ ID NO: 44 or SEQ ID NO: 45, at least 355 amino acids of SEQ ID NO: 47 or SEQ ID NO: 48, at least 345 amino acids of SEQ ID NO: 50 or SEQ ID NO: 51, at least 362 amino acids of SEQ ID NO: 53 or SEQ ID NO: 54, at least 450 amino acids of SEQ ID NO: 56 or SEQ ID NO: 57.

Fusion polypeptide: The term “fusion polypeptide” is a polypeptide in which one polypeptide is fused at the N-terminus or the C-terminus of a polypeptide of the present invention. A fusion polypeptide is produced by fusing a polynucleotide encoding another polypeptide to a polynucleotide of the present invention. Techniques for producing fusion polypeptides are known in the art, and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779). A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

Heterologous: The term “heterologous” means, with respect to a host cell, that a polypeptide or nucleic acid does not naturally occur in the host cell. The term “heterologous” means, with respect to a polypeptide or nucleic acid, that a control sequence, e.g., promoter, or domain of a polypeptide or nucleic acid is not naturally associated with the polypeptide or nucleic acid, i.e., the control sequence is from a gene other than the gene encoding the mature polypeptide of SEQ ID NO: 2, SEQ ID NO: 5, SEQ ID NO: 8, SEQ ID NO: 11, SEQ ID NO: 14, SEQ ID NO: 17, SEQ ID NO: 20, SEQ ID NO: 23, SEQ ID NO: 26, SEQ ID NO: 29, SEQ ID NO: 32, SEQ ID NO: 35, SEQ ID NO: 38, SEQ ID NO: 41, SEQ ID NO: 44, SEQ ID NO: 47, SEQ ID NO: 53, SEQ ID NO: 56.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Hybrid polypeptide: The term “hybrid polypeptide” means a polypeptide comprising domains from two or more polypeptides, e.g., a binding module from one polypeptide and a catalytic domain from another polypeptide. The domains may be fused at the N-terminus or the C-terminus.

Hybridization: The term “hybridization” means the pairing of substantially complementary strands of nucleic acids, using standard Southern blotting procedures. Hybridization may be performed under medium, medium-high, high or very high stringency conditions. Medium stringency conditions means prehybridization and hybridization at 42° C. in 5×SSPE, 0.3% SDS, 200 micrograms/ml sheared and denatured salmon sperm DNA, and 35% formamide for 12 to 24 hours, followed by washing three times each for 15 minutes using 0.2×SSC, 0.2% SDS at 55° C. Medium-high stringency conditions means prehybridization and hybridization at 42° C. in 5×SSPE, 0.3% SDS, 200 micrograms/ml sheared and denatured salmon sperm DNA, and 35% formamide for 12 to 24 hours, followed by washing three times each for 15 minutes using 0.2×SSC, 0.2% SDS at 60° C. High stringency conditions means prehybridization and hybridization at 42° C. in 5×SSPE, 0.3% SDS, 200 micrograms/ml sheared and denatured salmon sperm DNA, and 50% formamide for 12 to 24 hours, followed by washing three times each for 15 minutes using 0.2×SSC, 0.2% SDS at 65° C. Very high stringency conditions means prehybridization and hybridization at 42° C. in 5×SSPE, 0.3% SDS, 200 micrograms/ml sheared and denatured salmon sperm DNA, and 50% formamide for 12 to 24 hours, followed by washing three times each for 15 minutes using 0.2×SSC, 0.2% SDS at 70° C.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance). An isolated substance may be present in a fermentation broth sample; e.g. a host cell may be genetically modified to express the polypeptide of the invention. The fermentation broth from that host cell will comprise the isolated polypeptide.

Mature polypeptide: The term “mature polypeptide” means a polypeptide in its final form following translation and any post-translational modifications, such as N-terminal processing, C-terminal truncation, glycosylation, phosphorylation, etc.

In some aspects, the mature polypeptide is amino acids 1 to 353 of SEQ ID NO: 2 and amino acids −32 to −1 of SEQ ID NO: 2 is a signal peptide. In some aspects, the mature polypeptide is the amino acid sequence shown in SEQ ID NO: 3. In some aspects, the mature polypeptide is amino acids 1 to 349 of SEQ ID NO: 5 and amino acids −29 to −1 of SEQ ID NO: 5 is a signal peptide. In some aspects, the mature polypeptide is the amino acid sequence shown in SEQ ID NO: 6. In some aspects, the mature polypeptide is amino acids 1 to 350 of SEQ ID NO: 8 and amino acids −30 to −1 of SEQ ID NO: 8 is a signal peptide. In some aspects, the mature polypeptide is the amino acid sequence shown in SEQ ID NO: 9. In some aspects, the mature polypeptide is amino acids 1 to 479 of SEQ ID NO: 11 and amino acids −31 to −1 of SEQ ID NO: 11 is a signal peptide. In some aspects, the mature polypeptide is the amino acid sequence shown in SEQ ID NO: 12. In some aspects, the mature polypeptide is amino acids 1 to 489 of SEQ ID NO: 14 and amino acids −31 to −1 of SEQ ID NO: 14 is a signal peptide. In some aspects, the mature polypeptide is the amino acid sequence shown in SEQ ID NO: 15. In some aspects, the mature polypeptide is amino acids 1 to 348 of SEQ ID NO: 17 and amino acids −21 to −1 of SEQ ID NO: 17 is a signal peptide. In some aspects, the mature polypeptide is the amino acid sequence shown in SEQ ID NO: 18. In some aspects, the mature polypeptide is amino acids 1 to 538 of SEQ ID NO: 20 and amino acids −35 to −1 of SEQ ID NO: 20 is a signal peptide. In some aspects, the mature polypeptide is the amino acid sequence shown in SEQ ID NO: 21. In some aspects, the mature polypeptide is amino acids 1 to 542 of SEQ ID NO: 23 and amino acids −35 to −1 of SEQ ID NO: 23 is a signal peptide. In some aspects, the mature polypeptide is the amino acid sequence shown in SEQ ID NO: 24. In some aspects, the mature polypeptide is amino acids 1 to 1378 of SEQ ID NO: 26 and amino acids −33 to −1 of SEQ ID NO: 26 is a signal peptide. In some aspects, the mature polypeptide is the amino acid sequence shown in SEQ ID NO: 27. In some aspects, the mature polypeptide is amino acids 1 to 342 of SEQ ID NO: 29 and amino acids −22 to −1 of SEQ ID NO: 29 is a signal peptide. In some aspects, the mature polypeptide is the amino acid sequence shown in SEQ ID NO: 30. In some aspects, the mature polypeptide is amino acids 1 to 369 of SEQ ID NO: 32 and amino acids −18 to −1 of SEQ ID NO: 32 is a signal peptide. In some aspects, the mature polypeptide is the amino acid sequence shown in SEQ ID NO: 33. In some aspects, the mature polypeptide is amino acids 1 to 432 of SEQ ID NO: 35 and amino acids −25 to −1 of SEQ ID NO: 35 is a signal peptide. In some aspects, the mature polypeptide is the amino acid sequence shown in SEQ ID NO: 36. In some aspects, the mature polypeptide is amino acids xx of SEQ ID NO: 38 and amino acids −24 to −1 of SEQ ID NO: 38 is a signal peptide. In some aspects, the mature polypeptide is the amino acid sequence shown in SEQ ID NO: 39. In some aspects, the mature polypeptide is amino acids 1 to 380 of SEQ ID NO: 41 and amino acids −25 to −1 of SEQ ID NO: 41 is a signal peptide. In some aspects, the mature polypeptide is the amino acid sequence shown in SEQ ID NO: 42. In some aspects, the mature polypeptide is amino acids 1 to 392 of SEQ ID NO: 44 and amino acids −23 to −1 of SEQ ID NO: 44 is a signal peptide. In some aspects, the mature polypeptide is the amino acid sequence shown in SEQ ID NO: 45. In some aspects, the mature polypeptide is amino acids 1 to 395 of SEQ ID NO: 47 and amino acids −30 to −1 of SEQ ID NO: 47 is a signal peptide. In some aspects, the mature polypeptide is the amino acid sequence shown in SEQ ID NO: 48. In some aspects, the mature polypeptide is amino acids 1 to 384 of SEQ ID NO: 50 and amino acids −21 to −1 of SEQ ID NO: 50 is a signal peptide. In some aspects, the mature polypeptide is the amino acid sequence shown in SEQ ID NO: 51. In some aspects, the mature polypeptide is amino acids 1 to 403 of SEQ ID NO: 53 and amino acids −20 to −1 of SEQ ID NO: 53 is a signal peptide. In some aspects, the mature polypeptide is the amino acid sequence shown in SEQ ID NO: 54. In some aspects, the mature polypeptide is amino acids 1 to 500 of SEQ ID NO: 56 and amino acids −35 to −1 of SEQ ID NO: 56 is a signal peptide. In some aspects, the mature polypeptide is the amino acid sequence shown in SEQ ID NO: 57.

It is known in the art that a host cell may produce a mixture of two of more different mature polypeptides (i.e., with a different C-terminal and/or N-terminal amino acid) expressed by the same polynucleotide. It is also known in the art that different host cells process polypeptides differently, and thus, one host cell expressing a polynucleotide may produce a different mature polypeptide (e.g., having a different C-terminal and/or N-terminal amino acid) as compared to another host cell expressing the same polynucleotide.

Mature polypeptide coding sequence: The term “mature polypeptide coding sequence” means a polynucleotide that encodes a mature polypeptide having alpha-mannan degrading activity. In one aspect, the mature polypeptide coding sequence is nucleotides 97 to 1155 of SEQ ID NO: 1 and nucleotides 1 to 96 of SEQ ID NO: 1 encode a signal peptide. In one aspect, the mature polypeptide coding sequence is nucleotides 88 to 1134 of SEQ ID NO: 4 and nucleotides 1 to 87 encode a signal peptide. In one aspect, the mature polypeptide coding sequence is nucleotides 91 to 1140 of SEQ ID NO: 7 and nucleotides 1 to 90 encode a signal peptide. In one aspect, the mature polypeptide coding sequence is nucleotides 94 to 1530 of SEQ ID NO: 10 and nucleotides 1 to 93 encode a signal peptide. In one aspect, the mature polypeptide coding sequence is nucleotides 94 to 1560 of SEQ ID NO: 13 and nucleotides 1 to 93 encode a signal peptide. In one aspect, the mature polypeptide coding sequence is nucleotides 64 to 1107 of SEQ ID NO: 16, and nucleotides 1 to 63 encode a signal peptide. In one aspect, the mature polypeptide coding sequence is nucleotides 106 to 1719 of SEQ ID NO: 19, and nucleotides 1-105 encode a signal peptide. In one aspect, the mature polypeptide coding sequence is nucleotides 106 to 1731 of SEQ ID NO: 22, and nucleotides 1 to 105 encode a signal peptide. In one aspect, the mature polypeptide coding sequence is nucleotides 100 to 4236 of SEQ ID NO: 25, and nucleotides 1 to 99 encode a signal peptide. In one aspect, the mature polypeptide coding sequence is nucleotides 67 to 1092 of SEQ ID NO: 28, and nucleotides 1 to 66 encode a signal peptide. In one aspect, the mature polypeptide coding sequence is nucleotides 55 to 483 and 587 and 1264 of SEQ ID NO: 31, and nucleotides 1 to 54 encode a signal peptide. In one aspect, the mature polypeptide coding sequence is nucleotides 76 to 100, 159 to 853, and 905 to 1480 of SEQ ID NO: 34, and nucleotides 1 to 75 encode a signal peptide. In one aspect, the mature polypeptide coding sequence is nucleotides 73 to 97, 163 to 857, and 921 to 1304 of SEQ ID NO: 37, and nucleotides 1 to 72 encode a signal peptide. In one aspect, the mature polypeptide coding sequence is nucleotides 76 to 100, 233 to 477, 543 to 992, and 1054 to 1473 of SEQ ID NO: 40, and nucleotides 1 to 75 encode a signal peptide. In one aspect, the mature polypeptide coding sequence is nucleotides 70 to 100, 179 to 900, 967 to 1389 of SEQ ID NO: 43, and nucleotides 1 to 69 encode a signal peptide. In one aspect, the mature polypeptide coding sequence is nucleotides 91 to 118, 180 to 1336 of SEQ ID NO: 46, and nucleotides 1 to 90 encode a signal peptide. In one aspect, the mature polypeptide coding sequence is nucleotides 64 to 85, 161 to 640, 696 to 937, and 1015 to 1422 of SEQ ID NO: 49, and nucleotides 1 to 63 encode a signal peptide. In one aspect, the mature polypeptide coding sequence is nucleotides 61 to 85 and 154 to 1337 of SEQ ID NO: 52, and nucleotides 1 to 60 encode a signal peptide. In one aspect, the mature polypeptide coding sequence is nucleotides 106 to 1605 of SEQ ID NO: 55, and nucleotides 1 to 105 encode a signal peptide.

Native: The term “native” means a nucleic acid or polypeptide naturally occurring in a host cell.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Purified: The term “purified” means a nucleic acid or polypeptide that is substantially free from other components as determined by analytical techniques well known in the art (e.g., a purified polypeptide or nucleic acid may form a discrete band in an electrophoretic gel, chromatographic eluate, and/or a media subjected to density gradient centrifugation). A purified nucleic acid or polypeptide is at least about 50% pure, usually at least about 60%, about 65%, about 70%, about 75%, about 80%, about 85%, about 90%, about 91%, about 92%, about 93%, about 94%, about 95%, about 96%, about 97%, about 98%, about 99%, about 99.5%, about 99.6%, about 99.7%, about 99.8% or more pure (e.g., percent by weight on a molar basis). In a related sense, a composition is enriched for a molecule when there is a substantial increase in the concentration of the molecule after application of a purification or enrichment technique. The term “enriched” refers to a compound, polypeptide, cell, nucleic acid, amino acid, or other specified material or component that is present in a composition at a relative or absolute concentration that is higher than a starting composition.

Recombinant: The term “recombinant,” when used in reference to a cell, nucleic acid, protein or vector, means that it has been modified from its native state. Thus, for example, recombinant cells express genes that are not found within the native (non-recombinant) form of the cell, or express native genes at different levels or under different conditions than found in nature. Recombinant nucleic acids differ from a native sequence by one or more nucleotides and/or are operably linked to heterologous sequences, e.g., a heterologous promoter in an expression vector. Recombinant proteins may differ from a native sequence by one or more amino acids and/or are fused with heterologous sequences. A vector comprising a nucleic acid encoding a polypeptide is a recombinant vector. The term “recombinant” is synonymous with “genetically modified” and “transgenic”.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

$$( Identical Residues \times 100 ) / ( Length of Alignment - Total Number of Gaps in Alignment )$$

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

$$( Identical Deoxyribonucleotides \times 100 ) / ( Length of Alignment - Total Number of Gaps in Alignment )$$

Subsequence: The term “subsequence” means a polynucleotide having one or more (e.g., several) nucleotides absent from the 5′ and/or 3′ end of a mature polypeptide coding sequence; wherein the subsequence encodes a fragment having alpha-mannan degrading activity.

Variant: The term “variant” means a polypeptide having alpha-mannan degrading activity comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding an amino acid adjacent to and immediately following the amino acid occupying a position.

Wild-type: The term “wild-type” in reference to an amino acid sequence or nucleic acid sequence means that the amino acid sequence or nucleic acid sequence is a native or naturally-occurring sequence. As used herein, the term “naturally-occurring” refers to anything (e.g., proteins, amino acids, or nucleic acid sequences) that is found in nature. Conversely, the term “non-naturally occurring” refers to anything that is not found in nature (e.g., recombinant nucleic acids and protein sequences produced in the laboratory or modification of the wild-type sequence).

Biofilm: The term “biofilm” means any group of microorganisms in which cells stick to each other on a surface, such as a textile, dishware or hard surface. These adherent cells are frequently embedded within a self-produced matrix of extracellular polymeric substance (EPS). Biofilm EPS is a polymeric conglomeration generally composed of extracellular DNA, proteins, and polysaccharides.

Biofilms may form on living or non-living surfaces. The microbial cells growing in a biofilm are physiologically distinct from planktonic cells of the same organism, which, by contrast, are single-cells that may float or swim in a liquid medium.

Bacteria living in a biofilm usually have significantly different properties from free-floating bacteria of the same species, as the dense and protected environment of the film allows them to cooperate and interact in various ways. One effect of this environment is increased resistance to detergents and antibiotics, as the dense extracellular matrix and the outer layer of cells protect the interior of the community.

On laundry biofilm producing bacteria can be found among the following species: Acinetobacter sp., Aeromicrobium sp., Brevundimonas sp., Microbacterium sp., Micrococcus luteus, Staphylococcus epidermidis, Staphylococcus aureus, Pseudomonas sp., Pseudomonas aeruginosa, Pseudomonas alcaliphila, Pseudomonas fluorescens, Stenotrophomonas sp., Paraburkholderia, Burkolderia sp., Candida sp., Bordetella pertussis, Yersinia pestis, Escherichia coli and Aspergillus sp.

Cleaning component: The cleaning component e.g. a detergent adjunct ingredient is different to the polypeptides of this invention. The precise nature of these additional cleaning or adjunct components, and levels of incorporation thereof, will depend on the physical form of the composition and the nature of the operation for which it is to be used. Suitable cleaning components include, but are not limited to the components described below such as surfactants, builders, flocculating aid, chelating agents, dye transfer inhibitors, enzymes, enzyme stabilizers, enzyme inhibitors, catalytic materials, bleach activators, hydrogen peroxide, sources of hydrogen peroxide, preformed peracids, polymeric agents, clay soil removal/anti-redeposition agents, brighteners, suds suppressors, dyes, perfumes, structure elasticizing agents, fabric softeners, carriers, hydrotropes, builders and co-builders, fabric huing agents, anti-foaming agents, dispersants, processing aids, and/or pigments.

Cleaning Composition: The term cleaning composition includes “detergent composition” and refers to compositions that find use in the removal of undesired compounds from items to be cleaned, such as textiles. The detergent composition may be used to e.g. clean textiles for both household cleaning and industrial cleaning. The terms encompass any materials/compounds selected for the particular type of cleaning composition desired and the form of the product (e.g., liquid, gel, powder, granulate, paste, or spray compositions) and includes, but is not limited to, detergent compositions (e.g., liquid and/or solid laundry detergents and fine fabric detergents; fabric fresheners; fabric softeners; and textile and laundry pre-spotters/pretreatment). In addition to containing the enzyme of the invention, the detergent formulation may contain one or more additional enzymes (such as proteases, amylases, lipases, cutinases, cellulases, endoglucanases, xyloglucanases, pectinases, pectin lyases, xanthanases, peroxidases, haloperoxygenases, catalases and mannanases, or any mixture thereof), and/or detergent adjunct ingredients such as surfactants, builders, chelators or chelating agents, bleach system or bleach components, polymers, fabric conditioners, foam boosters, suds suppressors, dyes, perfume, tannish inhibitors, optical brighteners, bactericides, fungicides, soil suspending agents, anti-corrosion agents, enzyme inhibitors or stabilizers, enzyme activators, transferase(s), hydrolytic enzymes, oxido reductases, bluing agents and fluorescent dyes, antioxidants, and solubilizers.

Deep cleaning: The term “deep cleaning” means disruption, reduction or removal of organic components such as polysaccharides, proteins, DNA, soil or other components present in organic matter such as biofilm.

Delta remission value (ΔRem): The terms “Delta remission” or “Delta remission value” are defined herein as the result of a reflectance or remission measurement at a certain wavelength which typically is 460 nm. The swatch is measured with one swatch of similar colour as background, preferably a swatch from a repetition wash. A swatch representing each swatch type is measured before the wash. The Delta enzyme remission is the remission value of the swatch washed in detergent with an enzyme present minus the remission value of a similar swatch washed in a detergent without enzyme present.

Enzyme Detergency Benefit: The term “enzyme detergency benefit” is defined herein as the advantageous effect an enzyme may add to a detergent compared to the same detergent without the enzyme. Important detergency benefits which can be provided by enzymes are stain removal with no or very little visible soils after washing and/or cleaning, prevention or reduction of redeposition of soils released in the washing process (an effect that also is termed anti-redeposition), restoring fully or partly the whiteness of textiles which originally were white but after repeated use and wash have obtained a greyish or yellowish appearance (an effect that also is termed whitening). Textile care benefits, which are not directly related to catalytic stain removal or prevention of redeposition of soils, are also important for enzyme detergency benefits. Examples of such textile care benefits are prevention or reduction of dye transfer from one fabric to another fabric or another part of the same fabric (an effect that is also termed dye transfer inhibition or anti-backstaining), removal of protruding or broken fibers from a fabric surface to decrease pilling tendencies or remove already existing pills or fuzz (an effect that also is termed anti-pilling), improvement of the fabric-softness, colour clarification of the fabric and removal of particulate soils which are trapped in the fibers of the fabric or garment. Enzymatic bleaching is a further enzyme detergency benefit where the catalytic activity generally is used to catalyze the formation of bleaching components such as hydrogen peroxide or other peroxides.

Hard surface cleaning: The term “Hard surface cleaning” is defined herein as cleaning of hard surfaces wherein hard surfaces may include floors, tables, walls, roofs etc. as well as surfaces of hard objects such as cars (car wash) and dishes (dish wash). Dish washing includes but are not limited to cleaning of plates, cups, glasses, bowls, cutlery such as spoons, knives, forks, serving utensils, ceramics, plastics, metals, china, glass and acrylics.

Laundering: The term “laundering” relates to both household laundering and industrial laundering and means the process of treating textiles with a solution containing a cleaning or detergent composition of the present invention. The laundering process can for example be carried out using e.g. a household or an industrial washing machine or can be carried out by hand.

Malodor: The term “malodor” means an odor which is not desired on clean items. The cleaned item should smell fresh and clean without malodors adhered to the item. One example of malodor is compounds with an unpleasant smell, which may be produced by microorganisms. Another example is unpleasant smells can be sweat or body odor adhered to an item which has been in contact with human or animal. Another example of malodor can be the odor from spices, which sticks to items for example curry or other exotic spices which smells strongly.

Textile: The term “textile” means any textile material including yarns, yarn intermediates, fibers, non-woven materials, natural materials, synthetic materials, and any other textile material, fabrics made of these materials and products made from fabrics (e.g., garments and other articles). The textile or fabric may be in the form of knits, wovens, denims, non-wovens, felts, yarns, and towelling. The textile may be cellulose based such as natural cellulosics, including cotton, flax/linen, jute, ramie, sisal or coir or manmade cellulosics (e.g. originating from wood pulp) including viscose/rayon, cellulose acetate fibers (tricell), lyocell or blends thereof. The textile or fabric may also be non-cellulose based such as natural polyamides including wool, camel, cashmere, mohair, rabbit and silk or synthetic polymers such as nylon, aramid, polyester, acrylic, polypropylene and spandex/elastane, or blends thereof as well as blends of cellulose based and non-cellulose based fibers. Examples of blends are blends of cotton and/or rayon/viscose with one or more companion material such as wool, synthetic fiber (e.g. polyamide fiber, acrylic fiber, polyester fiber, polyvinyl chloride fiber, polyurethane fiber, polyurea fiber, aramid fiber), and/or cellulose-containing fiber (e.g. rayon/viscose, ramie, flax/linen, jute, cellulose acetate fiber, lyocell). Fabric may be conventional washable laundry, for example stained household laundry. When the term fabric or garment is used it is intended to include the broader term textiles as well.

Textile care benefit: Textile care benefits, which are not directly related to catalytic stain removal or prevention of redeposition of soils, are also important for enzyme detergency benefits. Examples of such textile care benefits are prevention or reduction of dye transfer from one textile to another textile or another part of the same textile (an effect that is also termed dye transfer inhibition or anti-backstaining), removal of protruding or broken fibers from a textile surface to decrease pilling tendencies or remove already existing pills or fuzz (an effect that also is termed anti-pilling), improvement of the textile-softness, colour clarification of the textile and removal of particulate soils which are trapped in the fibers of the textile. Enzymatic bleaching is a further enzyme detergency benefit where the catalytic activity generally is used to catalyze the formation of bleaching component such as hydrogen peroxide or other peroxides or other bleaching species.

Wash performance: The term “wash performance” is used as an enzyme's ability to remove stains present on the object to be cleaned during e.g. wash (laundry) or hard surface cleaning. The improvement in the wash performance may be quantified by calculating the so-called intensity value (Int) as described in the Examples herein. The term “improved wash performance” is defined herein as an enzyme displaying an increased wash performance in a cleaning composition relative to the wash performance of the same cleaning composition without the enzyme e.g. by increased stain removal or less re-deposition. The term “improved wash performance” includes wash performance in laundry but also e.g. in hard surface cleaning such as automated dish wash (ADW).

### DETAILED DESCRIPTION OF THE INVENTION

### Polypeptides Having Alpha-Mannan Degrading Activity

Polypeptides having alpha-mannan degrading activity or alpha-mannan degrading enzymes are enzymes having hydrolase activity on alpha-mannan. In particular, the polypeptide having alpha-mannan degrading activity includes enzymes from glycoside hydrolase domains GH76, GH92, and GH99, which are enzymes having alpha-mannanase and/or alpha-mannosidase activity. In an embodiment, the polypeptide belongs to GH family 76.

These can include enzyme activities such as alpha-1,6-mannanase (EC 3.2.1.101), alpha-1,2-mannase; mannosyl-oligosaccharide alpha-1,2-mannosidase (EC 3.2.1.113); mannosyl-oligosaccharide alpha-1,3-mannosidase (EC 3.2.1.-); mannosyl-oligosaccharide alpha-1,6-mannosidase (EC 3.2.1.-); alpha-mannosidase (EC 3.2.1.24); alpha-1,2-mannosidase (EC 3.2.1.-); alpha-1,3-mannosidase (EC 3.2.1.-); alpha-1,4-mannosidase (EC 3.2.1.-); mannosyl-1-phosphodiester alpha-1,P-mannosidase (EC 3.2.1.-); glycoprotein endo-alpha-1,2-mannosidase (EC 3.2.1.130); and/or mannan endo-1,2-alpha-mannanase (3.2.1.-) activities.

Also contemplated are blends of polypeptides having alpha-mannan degrading activity, including, for example, combinations of polypeptides having two or more different GH classifications according to the CAZY naming system.

In an embodiment are provided blends comprising a polypeptide belonging to GH family 76 and a polypeptide belonging to GH family 92; a polypeptide belonging to GH family 76 and a polypeptide belonging to GH family 99; a polypeptide belonging to GH family 92 and a polypeptide belonging to GH family 99; and in particular, a polypeptide belong to GH family 76 and a polypeptide belong to GH family 99.

Also contemplated are blends of three or more different GH classifications according to the CAZY naming system, including blends comprising a polypeptide belonging to GH family 76, a polypeptide belonging to GH family 92, and a polypeptide belonging to GH family 99. As also set forth in the Examples, a phylogenetic tree was constructed and various motifs have been identified. Thus, in one aspect, the polypeptide belongs to GH family 76, and comprises one or more of the motifs [YND]DD[QINLEM] (SEQ ID NO: 60) and GG[ILMV]X[WS] (SEQ ID NO: 61). Preferably, the polypeptide further comprises the motif [RK][NLT]XXX[NTV]XP[GTLYISAVFNM](SEQ ID NO: 64). In a more preferred aspect, the polypeptide is of the KNTPA clade and is of bacterial origin.

In another aspect, the polypeptide belongs to GH family 76, and comprises one or more of the motif GA]XX[AVL][ML]X[MA][ATV][EATV] (SEQ ID NO: 62) or the motif LA[EQ]X[VL][YF] (SEQ ID NO: 63). Preferably, the polypeptide is of the AMXAAE clade and is of fungal origin.

In another aspect, the polypeptide comprises the motif N[EQD][WFY][HG]E (SEQ ID NO: 66).

In some embodiments, the present invention relates to isolated or purified polypeptides selected from the group consisting of:
(a) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 3 or a fragment thereof having alpha-mannan degrading activity;
(b) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 6 or a fragment thereof having alpha-mannan degrading activity;
(c) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 9 or a fragment thereof having alpha-mannan degrading activity;
(d) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 12 or a fragment thereof having alpha-mannan degrading activity;
(e) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 15 or a fragment thereof having alpha-mannan degrading activity;
(f) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 18 or a fragment thereof having alpha-mannan degrading activity;
(g) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 21 or a fragment thereof having alpha-mannan degrading activity;
(h) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 24 or a fragment thereof having alpha-mannan degrading activity;
(i) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 27 or a fragment thereof having alpha-mannan degrading activity;
(j) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 30 or a fragment thereof having alpha-mannan degrading activity;
(k) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 33 or a fragment thereof having alpha-mannan degrading activity;
(l) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 36 or a fragment thereof having alpha-mannan degrading activity;
(m) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 39 or a fragment thereof having alpha-mannan degrading activity;
(n) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 42 or a fragment thereof having alpha-mannan degrading activity;
(o) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 45 or a fragment thereof having alpha-mannan degrading activity;
(p) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 48 or a fragment thereof having alpha-mannan degrading activity;
(q) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 51 or a fragment thereof having alpha-mannan degrading activity;
(r) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 54 or a fragment thereof having alpha-mannan degrading activity;
(s) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 57 or a fragment thereof having alpha-mannan degrading activity.

In some embodiments, including the proviso that the isolated or purified polypeptide is not the polypeptide of geneseqp:AXR38305 as described in WO 2009/108941 A2.

In some embodiments, the present invention relates to isolated or purified polypeptides belonging to family GH76 selected from the group consisting of:
(a) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 3 or a fragment thereof having alpha-mannan degrading activity;
(b) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 6 or a fragment thereof having alpha-mannan degrading activity;
(c) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 9 or a fragment thereof having alpha-mannan degrading activity;
(d) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 12 or a fragment thereof having alpha-mannan degrading activity;
(e) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 15 or a fragment thereof having alpha-mannan degrading activity;
(f) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 18 or a fragment thereof having alpha-mannan degrading activity;
(g) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 21 or a fragment thereof having alpha-mannan degrading activity;
(h) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 24 or a fragment thereof having alpha-mannan degrading activity;
(i) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 33 or a fragment thereof having alpha-mannan degrading activity;
(j) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 36 or a fragment thereof having alpha-mannan degrading activity;
(k) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 39 or a fragment thereof having alpha-mannan degrading activity;
(l) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 42 or a fragment thereof having alpha-mannan degrading activity;
(m) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 45 or a fragment thereof having alpha-mannan degrading activity;
(n) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 48 or a fragment thereof having alpha-mannan degrading activity;
(o) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 51 or a fragment thereof having alpha-mannan degrading activity;
(p) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 54 or a fragment thereof having alpha-mannan degrading activity;
(q) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 57 or a fragment thereof having alpha-mannan degrading activity.

In some embodiments, the present invention relates to isolated or purified polypeptides belonging to family GH92 selected from the group consisting of:
(a) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 27 or a fragment thereof having alpha-mannan degrading activity.

In some embodiments, the present invention relates to isolated or purified polypeptides belonging to family GH99 selected from the group consisting of:
(a) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 30 or a fragment thereof having alpha-mannan degrading activity.

In some embodiments, the present invention relates to isolated or purified polypeptides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide of SEQ ID NO: 2, which have alpha-mannan degrading activity. In one aspect, the polypeptides differ by up to 10 amino acids, e.g., 1, 2, 3, 4, 5, 6, 7, 8, 9, or 10, from the mature polypeptide of SEQ ID NO: 2.

The polypeptide preferably comprises, consists essentially of, or consists of the amino acid sequence of SEQ ID NO: 2 or the mature polypeptide thereof; or is a fragment thereof having alpha-mannan degrading activity.

In some embodiments, the present invention relates to isolated or purified polypeptides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide of SEQ ID NO: 5, which have alpha-mannan degrading activity. In one aspect, the polypeptides differ by up to 10 amino acids, e.g., 1, 2, 3, 4, 5, 6, 7, 8, 9, or 10, from the mature polypeptide of SEQ ID NO: 5.

The polypeptide preferably comprises, consists essentially of, or consists of the amino acid sequence of SEQ ID NO: 5 or the mature polypeptide thereof; or is a fragment thereof having alpha-mannan degrading activity.

In some embodiments, the present invention relates to isolated or purified polypeptides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide of SEQ ID NO: 8, which have alpha-mannan degrading activity. In one aspect, the polypeptides differ by up to 10 amino acids, e.g., 1, 2, 3, 4, 5, 6, 7, 8, 9, or 10, from the mature polypeptide of SEQ ID NO: 8.

The polypeptide preferably comprises, consists essentially of, or consists of the amino acid sequence of SEQ ID NO: 8 or the mature polypeptide thereof; or is a fragment thereof having alpha-mannan degrading activity.

In some embodiments, the present invention relates to isolated or purified polypeptides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide of SEQ ID NO: 11, which have alpha-mannan degrading activity. In one aspect, the polypeptides differ by up to 10 amino acids, e.g., 1, 2, 3, 4, 5, 6, 7, 8, 9, or 10, from the mature polypeptide of SEQ ID NO: 11.

The polypeptide preferably comprises, consists essentially of, or consists of the amino acid sequence of SEQ ID NO: 11 or the mature polypeptide thereof; or is a fragment thereof having alpha-mannan degrading activity.

In some embodiments, the present invention relates to isolated or purified polypeptides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide of SEQ ID NO: 14, which have alpha-mannan degrading activity. In one aspect, the polypeptides differ by up to 10 amino acids, e.g., 1, 2, 3, 4, 5, 6, 7, 8, 9, or 10, from the mature polypeptide of SEQ ID NO: 14.

The polypeptide preferably comprises, consists essentially of, or consists of the amino acid sequence of SEQ ID NO: 14 or the mature polypeptide thereof; or is a fragment thereof having alpha-mannan degrading activity.

In some embodiments, the present invention relates to isolated or purified polypeptides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide of SEQ ID NO: 17, which have alpha-mannan degrading activity. In one aspect, the polypeptides differ by up to 10 amino acids, e.g., 1, 2, 3, 4, 5, 6, 7, 8, 9, or 10, from the mature polypeptide of SEQ ID NO: 17.

The polypeptide preferably comprises, consists essentially of, or consists of the amino acid sequence of SEQ ID NO: 17 or the mature polypeptide thereof; or is a fragment thereof having alpha-mannan degrading activity.

In some embodiments, the present invention relates to isolated or purified polypeptides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide of SEQ ID NO: 20, which have alpha-mannan degrading activity. In one aspect, the polypeptides differ by up to 10 amino acids, e.g., 1, 2, 3, 4, 5, 6, 7, 8, 9, or 10, from the mature polypeptide of SEQ ID NO: 20.

The polypeptide preferably comprises, consists essentially of, or consists of the amino acid sequence of SEQ ID NO: 20 or the mature polypeptide thereof; or is a fragment thereof having alpha-mannan degrading activity.

In some embodiments, the present invention relates to isolated or purified polypeptides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide of SEQ ID NO: 23, which have alpha-mannan degrading activity. In one aspect, the polypeptides differ by up to 10 amino acids, e.g., 1, 2, 3, 4, 5, 6, 7, 8, 9, or 10, from the mature polypeptide of SEQ ID NO: 23.

The polypeptide preferably comprises, consists essentially of, or consists of the amino acid sequence of SEQ ID NO: 23 or the mature polypeptide thereof; or is a fragment thereof having alpha-mannan degrading activity.

In some embodiments, the present invention relates to isolated or purified polypeptides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide of SEQ ID NO: 26, which have alpha-mannan degrading activity. In one aspect, the polypeptides differ by up to 10 amino acids, e.g., 1, 2, 3, 4, 5, 6, 7, 8, 9, or 10, from the mature polypeptide of SEQ ID NO: 26.

The polypeptide preferably comprises, consists essentially of, or consists of the amino acid sequence of SEQ ID NO: 26 or the mature polypeptide thereof; or is a fragment thereof having alpha-mannan degrading activity.

In some embodiments, the present invention relates to isolated or purified polypeptides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide of SEQ ID NO: 29, which have alpha-mannan degrading activity. In one aspect, the polypeptides differ by up to 10 amino acids, e.g., 1, 2, 3, 4, 5, 6, 7, 8, 9, or 10, from the mature polypeptide of SEQ ID NO: 29.

The polypeptide preferably comprises, consists essentially of, or consists of the amino acid sequence of SEQ ID NO: 29 or the mature polypeptide thereof; or is a fragment thereof having alpha-mannan degrading activity.

In some embodiments, the present invention relates to isolated or purified polypeptides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide of SEQ ID NO: 32, which have alpha-mannan degrading activity. In one aspect, the polypeptides differ by up to 10 amino acids, e.g., 1, 2, 3, 4, 5, 6, 7, 8, 9, or 10, from the mature polypeptide of SEQ ID NO: 32.

The polypeptide preferably comprises, consists essentially of, or consists of the amino acid sequence of SEQ ID NO: 32 or the mature polypeptide thereof; or is a fragment thereof having alpha-mannan degrading activity.

In some embodiments, the present invention relates to isolated or purified polypeptides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide of SEQ ID NO: 35, which have alpha-mannan degrading activity. In one aspect, the polypeptides differ by up to 10 amino acids, e.g., 1, 2, 3, 4, 5, 6, 7, 8, 9, or 10, from the mature polypeptide of SEQ ID NO: 35.

The polypeptide preferably comprises, consists essentially of, or consists of the amino acid sequence of SEQ ID NO: 35 or the mature polypeptide thereof; or is a fragment thereof having alpha-mannan degrading activity.

In some embodiments, the present invention relates to isolated or purified polypeptides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide of SEQ ID NO: 38, which have alpha-mannan degrading activity. In one aspect, the polypeptides differ by up to 10 amino acids, e.g., 1, 2, 3, 4, 5, 6, 7, 8, 9, or 10, from the mature polypeptide of SEQ ID NO: 38.

The polypeptide preferably comprises, consists essentially of, or consists of the amino acid sequence of SEQ ID NO: 38 or the mature polypeptide thereof; or is a fragment thereof having alpha-mannan degrading activity.

In some embodiments, the present invention relates to isolated or purified polypeptides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide of SEQ ID NO: 41, which have alpha-mannan degrading activity. In one aspect, the polypeptides differ by up to 10 amino acids, e.g., 1, 2, 3, 4, 5, 6, 7, 8, 9, or 10, from the mature polypeptide of SEQ ID NO: 41.

The polypeptide preferably comprises, consists essentially of, or consists of the amino acid sequence of SEQ ID NO: 41 or the mature polypeptide thereof; or is a fragment thereof having alpha-mannan degrading activity.

In some embodiments, the present invention relates to isolated or purified polypeptides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide of SEQ ID NO: 44, which have alpha-mannan degrading activity. In one aspect, the polypeptides differ by up to 10 amino acids, e.g., 1, 2, 3, 4, 5, 6, 7, 8, 9, or 10, from the mature polypeptide of SEQ ID NO: 44.

The polypeptide preferably comprises, consists essentially of, or consists of the amino acid sequence of SEQ ID NO: 44 or the mature polypeptide thereof; or is a fragment thereof having alpha-mannan degrading activity.

In some embodiments, the present invention relates to isolated or purified polypeptides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide of SEQ ID NO: 47, which have alpha-mannan degrading activity. In one aspect, the polypeptides differ by up to 10 amino acids, e.g., 1, 2, 3, 4, 5, 6, 7, 8, 9, or 10, from the mature polypeptide of SEQ ID NO: 47.

The polypeptide preferably comprises, consists essentially of, or consists of the amino acid sequence of SEQ ID NO: 47 or the mature polypeptide thereof; or is a fragment thereof having alpha-mannan degrading activity.

In some embodiments, the present invention relates to isolated or purified polypeptides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide of SEQ ID NO: 50, which have alpha-mannan degrading activity. In one aspect, the polypeptides differ by up to 10 amino acids, e.g., 1, 2, 3, 4, 5, 6, 7, 8, 9, or 10, from the mature polypeptide of SEQ ID NO: 50.

The polypeptide preferably comprises, consists essentially of, or consists of the amino acid sequence of SEQ ID NO: 50 or the mature polypeptide thereof; or is a fragment thereof having alpha-mannan degrading activity.

In some embodiments, the present invention relates to isolated or purified polypeptides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide of SEQ ID NO: 53, which have alpha-mannan degrading activity. In one aspect, the polypeptides differ by up to 10 amino acids, e.g., 1, 2, 3, 4, 5, 6, 7, 8, 9, or 10, from the mature polypeptide of SEQ ID NO: 53.

The polypeptide preferably comprises, consists essentially of, or consists of the amino acid sequence of SEQ ID NO: 53 or the mature polypeptide thereof; or is a fragment thereof having alpha-mannan degrading activity.

In some embodiments, the present invention relates to isolated or purified polypeptides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide of SEQ ID NO: 56, which have alpha-mannan degrading activity. In one aspect, the polypeptides differ by up to 10 amino acids, e.g., 1, 2, 3, 4, 5, 6, 7, 8, 9, or 10, from the mature polypeptide of SEQ ID NO: 56.

The polypeptide preferably comprises, consists essentially of, or consists of the amino acid sequence of SEQ ID NO: 56 or the mature polypeptide thereof; or is a fragment thereof having alpha-mannan degrading activity.

In some embodiments, the present invention relates to isolated or purified polypeptides having alpha-mannan degrading activity encoded by polynucleotides that hybridize under medium stringency conditions, medium-high stringency conditions, high stringency conditions, or very high stringency conditions with the full-length complement of the mature polypeptide coding sequence of SEQ ID NO: 1 or the cDNA thereof (Sambrook et al., 1989, Molecular Cloning, A Laboratory Manual, 2d edition, Cold Spring Harbor, New York).

The polynucleotide of any of SEQ ID NO: 1, SEQ ID NO: 4, SEQ ID NO: 7, SEQ ID NO: 10, SEQ ID NO: 13, SEQ ID NO: 16, SEQ ID NO: 19, SEQ ID NO: 22, SEQ ID NO: 25, SEQ ID NO: 28, SEQ ID NO: 31, SEQ ID NO: 34, SEQ ID NO: 37, SEQ ID NO: 40, SEQ ID NO: 43, SEQ ID NO: 46, SEQ ID NO: 49, SEQ ID NO: 52, SEQ ID NO: 55, or a subsequence of any thereof, as well as the mature polypeptide of SEQ ID NO: 2, SEQ ID NO: 5, SEQ ID NO: 8, SEQ ID NO: 11, SEQ ID NO: 14, SEQ ID NO: 17, SEQ ID NO: 20, SEQ ID NO: 23, SEQ ID NO: 26, SEQ ID NO: 29, SEQ ID NO: 32, SEQ ID NO: 35, SEQ ID NO: 38, SEQ ID NO: 41, SEQ ID NO: 44, SEQ ID NO: 47, SEQ ID NO: 50, SEQ ID NO: 53, SEQ ID NO: 56 or a fragment of any thereof, may be used to design nucleic acid probes to identify and clone DNA encoding polypeptides having alpha-mannan degrading activity from strains of different genera or species according to methods well known in the art. Such probes can be used for hybridization with the genomic DNA or cDNA of a cell of interest, following standard Southern blotting procedures, in order to identify and isolate the corresponding gene therein. Such probes can be considerably shorter than the entire sequence, but should be at least 15, e.g., at least 25, at least 35, or at least 70 nucleotides in length. Preferably, the nucleic acid probe is at least 100 nucleotides in length, e.g., at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA or cDNA library prepared from such other strains may be screened for DNA that hybridizes with the probes described above and encodes a polypeptide having alpha-mannan degradi0ng activity. Genomic or other DNA from such other strains may be separated by agarose or polyacrylamide gel electrophoresis, or other separation techniques. DNA from the libraries or the separated DNA may be transferred to and immobilized on nitrocellulose or another suitable carrier material. In order to identify a clone or DNA that hybridizes with SEQ ID NO: 1 or a subsequence thereof, the carrier material is used in a Southern blot.

For purposes of the present invention, hybridization indicates that the polynucleotides hybridize to a labeled nucleic acid probe corresponding to (i) SEQ ID NO: 1; (ii) the mature polypeptide coding sequence of SEQ ID NO: 1; (iii) the full-length complement thereof; or (iv) a subsequence thereof; under medium to very high stringency conditions. Molecules to which the nucleic acid probe hybridizes under these conditions can be detected using, for example, X-ray film or any other detection means known in the art.

In some embodiments, the present invention relates to isolated polypeptides having alpha-mannan degrading activity encoded by polynucleotides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 1.

For purposes of the present invention, hybridization indicates that the polynucleotides hybridize to a labeled nucleic acid probe corresponding to (i) SEQ ID NO: 4; (ii) the mature polypeptide coding sequence of SEQ ID NO: 4; (iii) the full-length complement thereof; or (iv) a subsequence thereof; under medium to very high stringency conditions.

In some embodiments, the present invention relates to isolated polypeptides having alpha-mannan degrading activity encoded by polynucleotides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 4.

For purposes of the present invention, hybridization indicates that the polynucleotides hybridize to a labeled nucleic acid probe corresponding to (i) SEQ ID NO: 7; (ii) the mature polypeptide coding sequence of SEQ ID NO: 7; (iii) the full-length complement thereof; or (iv) a subsequence thereof; under medium to very high stringency conditions.

In some embodiments, the present invention relates to isolated polypeptides having alpha-mannan degrading activity encoded by polynucleotides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 7.

For purposes of the present invention, hybridization indicates that the polynucleotides hybridize to a labeled nucleic acid probe corresponding to (i) SEQ ID NO: 10; (ii) the mature polypeptide coding sequence of SEQ ID NO: 10; (iii) the full-length complement thereof; or (iv) a subsequence thereof; under medium to very high stringency conditions.

In some embodiments, the present invention relates to isolated polypeptides having alpha-mannan degrading activity encoded by polynucleotides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 10.

For purposes of the present invention, hybridization indicates that the polynucleotides hybridize to a labeled nucleic acid probe corresponding to (i) SEQ ID NO: 13; (ii) the mature polypeptide coding sequence of SEQ ID NO: 13; (iii) the full-length complement thereof; or (iv) a subsequence thereof; under medium to very high stringency conditions.

In some embodiments, the present invention relates to isolated polypeptides having alpha-mannan degrading activity encoded by polynucleotides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 13.

For purposes of the present invention, hybridization indicates that the polynucleotides hybridize to a labeled nucleic acid probe corresponding to (i) SEQ ID NO: 16; (ii) the mature polypeptide coding sequence of SEQ ID NO: 16; (iii) the full-length complement thereof; or (iv) a subsequence thereof; under medium to very high stringency conditions.

In some embodiments, the present invention relates to isolated polypeptides having alpha-mannan degrading activity encoded by polynucleotides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 16.

For purposes of the present invention, hybridization indicates that the polynucleotides hybridize to a labeled nucleic acid probe corresponding to (i) SEQ ID NO: 19; (ii) the mature polypeptide coding sequence of SEQ ID NO: 19; (iii) the full-length complement thereof; or (iv) a subsequence thereof; under medium to very high stringency conditions.

In some embodiments, the present invention relates to isolated polypeptides having alpha-mannan degrading activity encoded by polynucleotides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 19.

For purposes of the present invention, hybridization indicates that the polynucleotides hybridize to a labeled nucleic acid probe corresponding to (i) SEQ ID NO: 22; (ii) the mature polypeptide coding sequence of SEQ ID NO: 22; (iii) the full-length complement thereof; or (iv) a subsequence thereof; under medium to very high stringency conditions.

In some embodiments, the present invention relates to isolated polypeptides having alpha-mannan degrading activity encoded by polynucleotides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 22.

For purposes of the present invention, hybridization indicates that the polynucleotides hybridize to a labeled nucleic acid probe corresponding to (i) SEQ ID NO: 25; (ii) the mature polypeptide coding sequence of SEQ ID NO: 25; (iii) the full-length complement thereof; or (iv) a subsequence thereof; under medium to very high stringency conditions.

In some embodiments, the present invention relates to isolated polypeptides having alpha-mannan degrading activity encoded by polynucleotides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 25.

For purposes of the present invention, hybridization indicates that the polynucleotides hybridize to a labeled nucleic acid probe corresponding to (i) SEQ ID NO: 28; (ii) the mature polypeptide coding sequence of SEQ ID NO: 28; (iii) the full-length complement thereof; or (iv) a subsequence thereof; under medium to very high stringency conditions.

In some embodiments, the present invention relates to isolated polypeptides having alpha-mannan degrading activity encoded by polynucleotides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 28.

For purposes of the present invention, hybridization indicates that the polynucleotides hybridize to a labeled nucleic acid probe corresponding to (i) SEQ ID NO: 31; (ii) the mature polypeptide coding sequence of SEQ ID NO: 31; (iii) the cDNA sequence thereof; (iv) the full-length complement thereof; or (v) a subsequence thereof; under medium to very high stringency conditions.

In some embodiments, the present invention relates to isolated polypeptides having alpha-mannan degrading activity encoded by polynucleotides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 31, or the cDNA sequence thereof.

For purposes of the present invention, hybridization indicates that the polynucleotides hybridize to a labeled nucleic acid probe corresponding to (i) SEQ ID NO: 34; (ii) the mature polypeptide coding sequence of SEQ ID NO: 34; (iii) the cDNA sequence thereof; (iv) the full-length complement thereof; or (v) a subsequence thereof; under medium to very high stringency conditions.

In some embodiments, the present invention relates to isolated polypeptides having alpha-mannan degrading activity encoded by polynucleotides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 34, or the cDNA sequence thereof.

For purposes of the present invention, hybridization indicates that the polynucleotides hybridize to a labeled nucleic acid probe corresponding to (i) SEQ ID NO: 37; (ii) the mature polypeptide coding sequence of SEQ ID NO: 37; (iii) the cDNA sequence thereof; (iv) the full-length complement thereof; or (v) a subsequence thereof; under medium to very high stringency conditions.

In some embodiments, the present invention relates to isolated polypeptides having alpha-mannan degrading activity encoded by polynucleotides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 37, or the cDNA sequence thereof.

For purposes of the present invention, hybridization indicates that the polynucleotides hybridize to a labeled nucleic acid probe corresponding to (i) SEQ ID NO: 40; (ii) the mature polypeptide coding sequence of SEQ ID NO: 40; (iii) the cDNA sequence thereof; (iv) the full-length complement thereof; or (v) a subsequence thereof; under medium to very high stringency conditions.

In some embodiments, the present invention relates to isolated polypeptides having alpha-mannan degrading activity encoded by polynucleotides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 40, or the cDNA sequence thereof.

For purposes of the present invention, hybridization indicates that the polynucleotides hybridize to a labeled nucleic acid probe corresponding to (i) SEQ ID NO: 43; (ii) the mature polypeptide coding sequence of SEQ ID NO: 43; (iii) the cDNA sequence thereof; (iv) the full-length complement thereof; or (v) a subsequence thereof; under medium to very high stringency conditions.

In some embodiments, the present invention relates to isolated polypeptides having alpha-mannan degrading activity encoded by polynucleotides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 43, or the cDNA sequence thereof.

For purposes of the present invention, hybridization indicates that the polynucleotides hybridize to a labeled nucleic acid probe corresponding to (i) SEQ ID NO: 46; (ii) the mature polypeptide coding sequence of SEQ ID NO: 46; (iii) the cDNA sequence thereof; (iv) the full-length complement thereof; or (v) a subsequence thereof; under medium to very high stringency conditions.

In some embodiments, the present invention relates to isolated polypeptides having alpha-mannan degrading activity encoded by polynucleotides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 46, or the cDNA sequence thereof.

For purposes of the present invention, hybridization indicates that the polynucleotides hybridize to a labeled nucleic acid probe corresponding to (i) SEQ ID NO: 49; (ii) the mature polypeptide coding sequence of SEQ ID NO: 49; (iii) the cDNA sequence thereof; (iv) the full-length complement thereof; or (v) a subsequence thereof; under medium to very high stringency conditions.

In some embodiments, the present invention relates to isolated polypeptides having alpha-mannan degrading activity encoded by polynucleotides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 49, or the cDNA sequence thereof.

For purposes of the present invention, hybridization indicates that the polynucleotides hybridize to a labeled nucleic acid probe corresponding to (i) SEQ ID NO: 52; (ii) the mature polypeptide coding sequence of SEQ ID NO: 52; (iii) the cDNA sequence thereof; (iv) the full-length complement thereof; or (v) a subsequence thereof; under medium to very high stringency conditions.

In some embodiments, the present invention relates to isolated polypeptides having alpha-mannan degrading activity encoded by polynucleotides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 52, or the cDNA sequence thereof.

For purposes of the present invention, hybridization indicates that the polynucleotides hybridize to a labeled nucleic acid probe corresponding to (i) SEQ ID NO: 55; (ii) the mature polypeptide coding sequence of SEQ ID NO: 55; (iii) the full-length complement thereof; or (iv) a subsequence thereof; under medium to very high stringency conditions. Molecules to which the nucleic acid probe hybridizes under these conditions can be detected using, for example, X-ray film or any other detection means known in the art.

In some embodiments, the present invention relates to isolated polypeptides having alpha-mannan degrading activity encoded by polynucleotides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 55.

In some embodiments, the present invention relates to variants of the mature polypeptide of SEQ ID NO: 2, SEQ ID NO: 5, SEQ ID NO: 8, SEQ ID NO: 11, SEQ ID NO: 14, SEQ ID NO: 17, SEQ ID NO: 20, SEQ ID NO: 23, SEQ ID NO: 26, SEQ ID NO: 29, SEQ ID NO: 32, SEQ ID NO: 35, SEQ ID NO: 38, SEQ ID NO: 41, SEQ ID NO: 44, SEQ ID NO: 47, SEQ ID NO: 50, SEQ ID NO: 53, or SEQ ID NO: 56 comprising a substitution, deletion, and/or insertion at one or more (e.g., several) positions. In one aspect, the number of amino acid substitutions, deletions and/or insertions introduced into the mature polypeptide of SEQ ID NO: 2, SEQ ID NO: 5, SEQ ID NO: 8, SEQ ID NO: 11, SEQ ID NO: 14, SEQ ID NO: 17, SEQ ID NO: 20, SEQ ID NO: 23, SEQ ID NO: 26, SEQ ID NO: 29, SEQ ID NO: 32, SEQ ID NO: 35, SEQ ID NO: 38, SEQ ID NO: 41, SEQ ID NO: 44, SEQ ID NO: 47, SEQ ID NO: 50, SEQ ID NO: 53, or SEQ ID NO: 56 is up to 10, e.g., 1, 2, 3, 4, 5, 6, 7, 8, 9, or 10. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module.

Essential amino acids in a polypeptide can be identified according to procedures known in the art, such as site-directed mutagenesis or alanine-scanning mutagenesis (Cunningham and Wells, 1989, 244: 1081-1085). In the latter technique, single alanine mutations are introduced at every residue in the molecule, and the resultant molecules are tested for alpha-mannan degrading activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

The polypeptide may be a hybrid polypeptide or a fusion polypeptide.

### Polypeptides Having Beta-Mannanase Activity

The beta-mannanases suitable for use according to the compositions of the present invention include polypeptides having beta-mannanase enzyme defined as the officially named mannan endo-1,4-beta-mannosidase and having the alternative names beta-mannanase and endo-1,4-mannase. The beta-mannanase term also means a polypeptide or polypeptide domain of an enzymes that has the ability to catalyze the cleavage or hydrolysis of (1->4) beta-D-mannosidic linkages of mannans, galactomannans, glucomannans, and galactoglucmannans. Thus, it means that the beta-mannanase has beta-mannanase activity (EC 3.2.1.78).

Suitable beta-mannanases include those of bacterial or fungal origin. Chemically or genetically modified mutants are included. The mannanase may be an alkaline beta-mannanase of Family 5 or 26. It may be a wild-type from Bacillus or Humicola, particularly B. agaradhaerens, B. licheniformis, B. halodurans, B. clausii, or H. insolens. Suitable GH5 beta-mannanases are described in WO 1999/064619, WO 2014/088940, WO 2014/100018, WO 2016/007929. Suitable GH26 beta-mannanases are described in WO 2012/149317, WO 2012/149325, WO 2012/149333, WO 2015/144782, WO 2016/054176, WO 2017/021514, WO 2017/021515, WO 2017/021516, WO 2017/021517, WO 2017/021518. A commercially available beta-mannanase is Mannaway (Novozymes A/S).

In an embodiment, the amino acid sequence of the beta-mannanase has at least 59%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of a beta-mannanase obtainable from Bacillus bogoriensis shown in SEQ ID NO: 67. In one aspect, the beta-mannanase comprises, consists, or consists essentially of the amino acid sequence set forth in SEQ ID NO: 67.

In an embodiment, the amino acid sequence of the beta-mannanase has at least 59%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of a beta-mannanase obtainable from Paenibacillus sp. (PspMan4) shown in SEQ ID NO: 68. In one aspect, the beta-mannanase comprises, consists, or consists essentially of the amino acid sequence set forth in SEQ ID NO: 68.

In an embodiment, the amino acid sequence of the beta-mannanase has at least 59%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to the amino acid sequence of a beta-mannanase obtainable from Bacillus hemicellulosilyticus shown in SEQ ID NO: 69. In one aspect, the beta-mannanase comprises, consists, or consists essentially of the amino acid sequence set forth in SEQ ID NO: 69.

The concentration of the beta-mannanase is preferably in the range of below 2 ppm enzyme protein, such as at from 0.01 ppm-2.0 ppm, 0.05 ppm-2.0 ppm, 0.1 ppm-2.0 ppm, 0.1 ppm-1.5 ppm, 0.1 ppm-1.0 ppm, 0.2 ppm-2.0 ppm, 0.5 ppm-2.0 ppm, 0.5 ppm-1.5 ppm or preferably at a concentration between 0.5 ppm and 1.0 ppm.

### Sources of Polypeptides Having Alpha-Mannan Degrading Activity

A polypeptide having alpha-mannan degrading activity of the present invention may be obtained from microorganisms of any genus. For purposes of the present invention, the term “obtained from” as used herein in connection with a given source shall mean that the polypeptide encoded by a polynucleotide is produced by the source or by a strain in which the polynucleotide from the source has been inserted. In one aspect, the polypeptide obtained from a given source is secreted extracellularly.

In another aspect, the polypeptide is a Bacillus polypeptide, e.g., a polypeptide obtained from Bacillus acidicola, Bacillus novalis, or Bacillus sp. In another aspect, the polypeptide is a Paenibacillus polypeptide, e.g., a polypeptide obtained from Paenibacillus glycanilyticus, or Paenibacillus sp.

In another aspect, the polypeptide is an Aspergillus polypeptide, e.g., a polypeptide obtained from Aspergillus aculeatus. In another aspect, the polypeptide is a Humicola polyeptide, e.g., a polypeptide obtained from Humicola insolens.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen und Zellkulturen GmbH (DSMZ), Centraalbureau Voor Schimmelcultures (CBS), and Agricultural Research Service Patent Culture Collection, Northern Regional Research Center (NRRL).

The polypeptides may be identified and obtained from other sources including microorganisms isolated from nature (e.g., soil, composts, water, etc.) or DNA samples obtained directly from natural materials (e.g., soil, composts, water, etc.) using the above-mentioned probes.

Techniques for isolating microorganisms and DNA directly from natural habitats are well known in the art. A polynucleotide encoding the polypeptide may then be obtained by similarly screening a genomic DNA or cDNA library of another microorganism or mixed DNA sample. Once a polynucleotide encoding a polypeptide has been detected with the probe(s), the polynucleotide can be isolated or cloned by utilizing techniques that are known to those of ordinary skill in the art (see, e.g., Sambrook et al., 1989, supra).

### Polynucleotides

The present invention also relates to isolated polynucleotides encoding a polypeptide, a catalytic domain, or carbohydrate binding module of the present invention, as described herein.

The techniques used to isolate or clone a polynucleotide are known in the art and include isolation from genomic DNA or cDNA, or a combination thereof. The cloning of the polynucleotides from genomic DNA can be effected, e.g., by using the polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and polynucleotide-based amplification (NASBA) may be used. The polynucleotides may be cloned from a strain of Bacillus, e.g., Bacillus acidicola, Bacillus novalis, or Bacillus sp., a strain of Paenibacillus, e.g., Paenibacillus glycanilyticus, or Paenibacillus sp., or from a strain of Aspergillus, e.g., Aspergillus aculeatus, or from Humicola, e.g., Humicola insolens, or a related organism and thus, for example, may be a species variant of the polypeptide encoding region of the polynucleotide.

Modification of a polynucleotide encoding a polypeptide of the present invention may be necessary for synthesizing polypeptides substantially similar to the polypeptide. The term “substantially similar” to the polypeptide refers to non-naturally occurring forms of the polypeptide. These polypeptides may differ in some engineered way from the polypeptide isolated from its native source, e.g., variants that differ in specific activity, thermostability, pH optimum, or the like. The variants may be constructed on the basis of the polynucleotide presented as the mature polypeptide coding sequence of SEQ ID NO: 1, SEQ ID NO: 4, SEQ ID NO: 7, SEQ ID NO: 10, SEQ ID NO: 13, SEQ ID NO: 16, SEQ ID NO: 19, SEQ ID NO: 22, SEQ ID NO: 25, SEQ ID NO: 28, SEQ ID NO: 31 or the cDNA sequence thereof, SEQ ID NO: 34 or the cDNA sequence thereof, SEQ ID NO: 37 or the cDNA sequence thereof, SEQ ID NO: 40 or the cDNA sequence thereof, SEQ ID NO: 43 or the cDNA sequence thereof, SEQ ID NO: 46 or the cDNA sequence thereof, SEQ ID NO: 49 or the cDNA sequence thereof, SEQ ID NO: 52 or the cDNA sequence thereof, or SEQ ID NO: 55 or the cDNA sequence thereof, e.g., a subsequence thereof, and/or by introduction of nucleotide substitutions that do not result in a change in the amino acid sequence of the polypeptide, but which correspond to the codon usage of the host organism intended for production of the enzyme, or by introduction of nucleotide substitutions that may give rise to a different amino acid sequence. For a general description of nucleotide substitution, see, e.g., Ford et al., 1991, Protein Expression and Purification 2: 95-107.

### Nucleic Acid Constructs

The present invention also relates to nucleic acid constructs comprising a polynucleotide of the present invention, wherein the polynucleotide is operably linked to one or more control sequences that direct the expression of the coding sequence in a suitable host cell under conditions compatible with the control sequences.

The polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the polynucleotide of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, Bacillus thuringiensis cryIIIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

Examples of suitable promoters for directing transcription of the polynucleotide of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase Ill, Trichoderma reesei endoglucanase V, Trichoderma reesei xylanase I, Trichoderma reesei xylanase II, Trichoderma reesei xylanase III, Trichoderma reesei beta-xylosidase, and Trichoderma reeseitranslation elongation factor, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and mutant, truncated, and hybrid promoters thereof. Other promoters are described in U.S. Pat. No. 6,011,147.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase Ill, Trichoderma reesei endoglucanase V, Trichoderma reesei xylanase I, Trichoderma reesei xylanase II, Trichoderma reesei xylanase Ill, Trichoderma reesei beta-xylosidase, and Trichoderma reesei translation elongation factor.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, J. Bacteriol. 177: 3465-3471).

The control sequence may also be a leader, a nontranslated region of an mRNA that is important for translation by the host cell. The leader is operably linked to the 5′-terminus of the polynucleotide encoding the polypeptide. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is heterologous to the coding sequence. A heterologous signal peptide coding sequence may be required where the coding sequence does not naturally contain a signal peptide coding sequence. Alternatively, a heterologous signal peptide coding sequence may simply replace the natural signal peptide coding sequence to enhance secretion of the polypeptide. However, any signal peptide coding sequence that directs the expressed polypeptide into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiol. Rev. 57: 109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked to the regulatory sequence.

### Expression Vectors

The present invention also relates to recombinant expression vectors comprising a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, adeA (phosphoribosylaminoimidazole-succinocarboxamide synthase), adeB (phosphoribosyl-aminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is a hph-tk dual selectable marker system.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMβ1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANS1 (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells, comprising a polynucleotide of the present invention operably linked to one or more control sequences that direct the production of a polypeptide of the present invention. A construct or vector comprising a polynucleotide is introduced into a host cell so that the construct or vector is maintained as a chromosomal integrant or as a self-replicating extra-chromosomal vector as described earlier. The choice of a host cell will to a large extent depend upon the gene encoding the polypeptide and its source.

In some embodiments, the polypeptide is heterologous to the recombinant host cell.

In some embodiments, at least one of the one or more control sequences is heterologous to the polynucleotide encoding the polypeptide.

In some embodiments, the recombinant host cell comprises at least two copies, e.g., three, four, or five, of the polynucleotide of the present invention.

The host cell may be any microbial or plant cell useful in the recombinant production of a polypeptide of the present invention, e.g., a prokaryotic cell or a fungal cell.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397) or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804), or conjugation (see, e.g., Clewell, 1981, Microbiol. Rev. 45: 409-436). However, any method known in the art for introducing DNA into a host cell can be used.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Talaromyces emersonii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75:1920.

### Methods of Production

The present invention also relates to methods of producing a polypeptide of the present invention, comprising (a) cultivating a cell, which in its wild-type form produces the polypeptide, under conditions conducive for production of the polypeptide; and optionally, (b) recovering the polypeptide. In one aspect, the cell is a Bacillus or Paenibacillus or Aspergillus or Humicola cell. In another aspect, the cell is a Bacillus acidicola, Bacillus novalis, or Bacillus sp., Paenibacillus glycanilyticus, or Paenibacillus sp., or Aspergillus aculeatus, or Humicola insolens, cell.

The present invention also relates to methods of producing a polypeptide of the present invention, comprising (a) cultivating a recombinant host cell of the present invention under conditions conducive for production of the polypeptide; and optionally, (b) recovering the polypeptide.

The host cells are cultivated in a nutrient medium suitable for production of the polypeptide using methods known in the art. For example, the cells may be cultivated by shake flask cultivation, or small-scale or large-scale fermentation (including continuous, batch, fed-batch, or solid-state fermentations) in laboratory or industrial fermentors in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptides. These detection methods include, but are not limited to, use of specific antibodies, formation of an enzyme product, or disappearance of an enzyme substrate. For example, an enzyme assay may be used to determine the activity of the polypeptide

The polypeptide may be recovered using methods known in the art. For example, the polypeptide may be recovered from the fermentation medium by conventional procedures including, but not limited to, collection, centrifugation, filtration, extraction, spray-drying, evaporation, or precipitation. In one aspect, a whole fermentation broth comprising the polypeptide is recovered.

The polypeptide may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

### Fermentation Broth Formulations or Cell Compositions

The present invention also relates to a fermentation broth formulation or a cell composition comprising a polypeptide of the present invention. The fermentation broth formulation or the cell composition further comprises additional ingredients used in the fermentation process, such as, for example, cells (including, the host cells containing the gene encoding the polypeptide of the present invention which are used to produce the polypeptide of interest), cell debris, biomass, fermentation media and/or fermentation products. In some embodiments, the composition is a cell-killed whole broth containing organic acid(s), killed cells and/or cell debris, and culture medium.

The term “fermentation broth” as used herein refers to a preparation produced by cellular fermentation that undergoes no or minimal recovery and/or purification. For example, fermentation broths are produced when microbial cultures are grown to saturation, incubated under carbon-limiting conditions to allow protein synthesis (e.g., expression of enzymes by host cells) and secretion into cell culture medium. The fermentation broth can contain unfractionated or fractionated contents of the fermentation materials derived at the end of the fermentation. Typically, the fermentation broth is unfractionated and comprises the spent culture medium and cell debris present after the microbial cells (e.g., filamentous fungal cells) are removed, e.g., by centrifugation. In some embodiments, the fermentation broth contains spent cell culture medium, extracellular enzymes, and viable and/or nonviable microbial cells.

In some embodiments, the fermentation broth formulation or the cell composition comprises a first organic acid component comprising at least one 1-5 carbon organic acid and/or a salt thereof and a second organic acid component comprising at least one 6 or more carbon organic acid and/or a salt thereof. In some embodiments, the first organic acid component is acetic acid, formic acid, propionic acid, a salt thereof, or a mixture of two or more of the foregoing and the second organic acid component is benzoic acid, cyclohexanecarboxylic acid, 4-methylvaleric acid, phenylacetic acid, a salt thereof, or a mixture of two or more of the foregoing.

In one aspect, the composition contains an organic acid(s), and optionally further contains killed cells and/or cell debris. In some embodiments, the killed cells and/or cell debris are removed from a cell-killed whole broth to provide a composition that is free of these components.

The fermentation broth formulation or cell composition may further comprise a preservative and/or anti-microbial (e.g., bacteriostatic) agent, including, but not limited to, sorbitol, sodium chloride, potassium sorbate, and others known in the art.

The cell-killed whole broth or composition may contain the unfractionated contents of the fermentation materials derived at the end of the fermentation. Typically, the cell-killed whole broth or composition contains the spent culture medium and cell debris present after the microbial cells (e.g., filamentous fungal cells) are grown to saturation, incubated under carbon-limiting conditions to allow protein synthesis. In some embodiments, the cell-killed whole broth or composition contains the spent cell culture medium, extracellular enzymes, and killed filamentous fungal cells. In some embodiments, the microbial cells present in the cell-killed whole broth or composition can be permeabilized and/or lysed using methods known in the art.

A whole broth or cell composition as described herein is typically a liquid, but may contain insoluble components, such as killed cells, cell debris, culture media components, and/or insoluble enzyme(s). In some embodiments, insoluble components may be removed to provide a clarified liquid composition.

The whole broth formulations and cell composition of the present invention may be produced by a method described in WO 90/15861 or WO 2010/096673.

### Enzyme Compositions

In an embodiment, the invention relates to compositions, in particular, cleaning compositions such as a detergent composition, comprising a polypeptide having alpha-mannan degrading activity in combination with one or more additional component(s). The choice of additional components is within the skill of the artisan and includes conventional ingredients, including the exemplary non-limiting components set forth below.

The compositions may be prepared in accordance with methods known in the art and may be in the form of a liquid or a dry composition. The compositions may be stabilized in accordance with methods known in the art.

Examples are given below of preferred uses of the compositions of the present invention. The dosage of the composition and other conditions under which the composition is used may be determined on the basis of methods known in the art.

In one embodiment of the present invention, the polypeptide having alpha-mannan degrading activity may be added to a cleaning composition, such as a detergent composition, in an amount corresponding to 0.001-200 mg of protein, such as 0.005-100 mg of protein, preferably 0.01-50 mg of protein, more preferably 0.05-20 mg of protein, even more preferably 0.1-10 mg of protein per liter of wash liquor.

The polypeptide having alpha-mannan degrading activity may be stabilized for inclusion in a composition using conventional stabilizing agents, e.g. a polyol such as propylene glycol or glycerol, a sugar or sugar alcohol, lactic acid, boric acid, or a boric acid derivative, e.g. an aromatic borate ester, or a phenyl boronic acid derivative such as 4-formylphenyl boronic acid, and the composition may be formulated as described in, for example, WO92/19709 and WO92/19708.

A polypeptide having alpha-mannan degrading activity may also be incorporated in the detergent formulations disclosed in WO97/07202, which is hereby incorporated by reference.

In one embodiment, the invention is directed to detergent compositions comprising an The polypeptide having alpha-mannan degrading activity in combination with one or more additional cleaning composition components. The choice of additional components is within the skill of the artisan and includes conventional ingredients, including the exemplary non-limiting components set forth below.

The choice of components may include, for textile care, the consideration of the type of textile to be cleaned, the type and/or degree of soiling, the temperature at which cleaning is to take place, and the formulation of the detergent product. Although components mentioned below are categorized by general header according to a particular functionality, this is not to be construed as a limitation, as a component may comprise additional functionalities as will be appreciated by the skilled artisan.

In one embodiment, the invention is directed to an ADW (Automatic Dish Wash) compositions comprising an enzyme of the present invention in combination with one or more additional ADW composition components. The choice of additional components is within the skill of the artisan and includes conventional ingredients, including the exemplary non-limiting components set forth below.

Some embodiments of the invention relate to a composition comprising:
a) at least 0.001 ppm of at least one polypeptide having alpha-mannan degrading activity, wherein the polypeptide is selected for the group consisting of: a polypeptide having at least 60%, at least 65%, at least 70%, at least 75%, at least 80%, at least 85%, at least 90%, at least 95%, at least 96%, at least 97%, at least 98%, at least 99% or 100% identity to the polypeptide of SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO; 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 54, or SEQ ID NO: 57;
b) one or more cleaning components.

Some embodiments of the invention relate to a cleaning composition comprising:
a) at least 0.001 ppm of at least one polypeptide having alpha-mannan degrading activity, wherein the polypeptide is selected for the group consisting of: a polypeptide having at least 60%, at least 65%, at least 70%, at least 75%, at least 80%, at least 85%, at least 90%, at least 95%, at least 96%, at least 97%, at least 98%, at least 99% or 100% identity to the polypeptide of SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO; 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 54, or SEQ ID NO: 57;
b) at least one cleaning component, preferably selected from a surfactant, a builder, a bleach component, a polymer, a dispersing agent and/or an additional enzyme.

One embodiment relates to a cleaning composition comprising:
a) at least 0.001 ppm of at least one polypeptide having alpha-mannan degrading activity e.g. a glycosyl hydrolase family 76, glycosyl hydrolase family 92, or glycosyl hydrolase family 99, preferably a glycosyl hydrolase family 76, wherein the polypeptide is selected from the group consisting of:
   i) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 3;
   ii) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 6;
   iii) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 9;
   iv) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO:12;
   v) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 15;
   vi) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 18;
   vii) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 21;
   viii) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 24;
   ix) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 27;
   x) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 30;
   xi) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 33;
   xii) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 36;
   xiii) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 39;
   xiv) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 42;
   xv) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 45;
   xvi) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 48;
   xvii) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 51;
   xviii) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 54;
   xix) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 57, and
b) at least one cleaning component, preferably selected from a surfactant, a builder, a bleach component, a polymer, a dispersing agent and/or an additional enzyme.

### Surfactants

The detergent composition may comprise one or more surfactants, which may be anionic and/or cationic and/or non-ionic and/or semi-polar and/or zwitterionic, or a mixture thereof. In a particular embodiment, the detergent composition includes a mixture of one or more nonionic surfactants and one or more anionic surfactants. The surfactant(s) is typically present at a level of from about 0.1% to 60% by weight, such as about 1% to about 40%, or about 3% to about 20%, or about 3% to about 10%. The surfactant(s) is chosen based on the desired cleaning application, and may include any conventional surfactant(s) known in the art.

When included therein the detergent will usually contain from about 1% to about 40% by weight of an anionic surfactant, such as from about 5% to about 30%, including from about 5% to about 15%, or from about 15% to about 20%, or from about 20% to about 25% of an anionic surfactant. Non-limiting examples of anionic surfactants include sulfates and sulfonates, in particular, linear alkylbenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ethersulfates (AES or AEOS or FES, also known as alcohol ethoxysulfates or fatty alcohol ether sulfates), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or salt of fatty acids (soap), and combinations thereof.

When included therein the detergent will usually contain from about 1% to about 40% by weigh of a cationic surfactant, for example from about 0.5% to about 30%, in particular from about 1% to about 20%, from about 3% to about 10%, such as from about 3% to about 5%, from about 8% to about 12% or from about 10% to about 12%. Non-limiting examples of cationic surfactants include alkyldimethylethanolamine quat (ADMEAQ), cetyltrimethylammonium bromide (CTAB), dimethyldistearylammonium chloride (DSDMAC), and alkylbenzyldimethylammonium, alkyl quaternary ammonium compounds, alkoxylated quaternary ammonium (AQA) compounds, ester quats, and combinations thereof.

When included therein the detergent will usually contain from about 0.2% to about 40% by weight of a nonionic surfactant, for example from about 0.5% to about 30%, in particular from about 1% to about 20%, from about 3% to about 10%, such as from about 3% to about 5%, from about 8% to about 12%, or from about 10% to about 12%. Non-limiting examples of nonionic surfactants include alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxyalkyl fatty acid amides, or N-acyl N-alkyl derivatives of glucosamine (glucamides, GA, or fatty acid glucamides, FAGA), as well as products available under the trade names SPAN and TWEEN, and combinations thereof.

Non-limiting examples of semipolar surfactants include amine oxides (AO) such as alkyldimethylamineoxide, N-(coco alkyl)-N,N-dimethylamine oxide and N-(tallow-alkyl)-N,N-bis(2-hydroxyethyl)amine oxide, and combinations thereof.

Non-limiting examples of zwitterionic surfactants include betaines such as alkyldimethylbetaines, sulfobetaines, and combinations thereof.

### Hydrotropes

A hydrotrope is a compound that solubilises hydrophobic compounds in aqueous solutions (or oppositely, polar substances in a non-polar environment). Typically, hydrotropes have both hydrophilic and a hydrophobic character (so-called amphiphilic properties as known from surfactants); however the molecular structure of hydrotropes generally do not favor spontaneous self-aggregation, see e.g. review by Hodgdon and Kaler (2007), Current Opinion in Colloid & Interface Science 12: 121-128. Hydrotropes do not display a critical concentration above which self-aggregation occurs as found for surfactants and lipids forming miceller, lamellar or other well defined meso-phases. Instead, many hydrotropes show a continuous-type aggregation process where the sizes of aggregates grow as concentration increases. However, many hydrotropes alter the phase behavior, stability, and colloidal properties of systems containing substances of polar and non-polar character, including mixtures of water, oil, surfactants, and polymers. Hydrotropes are classically used across industries from pharma, personal care, food, to technical applications. Use of hydrotropes in detergent compositions allow for example more concentrated formulations of surfactants (as in the process of compacting liquid detergents by removing water) without inducing undesired phenomena such as phase separation or high viscosity.

The detergent may contain 0-10% by weight, for example 0-5% by weight, such as about 0.5 to about 5%, or about 3% to about 5%, of a hydrotrope. Any hydrotrope known in the art for use in detergents may be utilized. Non-limiting examples of hydrotropes include sodium benzenesulfonate, sodium p-toluene sulfonate (STS), sodium xylene sulfonate (SXS), sodium cumene sulfonate (SCS), sodium cymene sulfonate, amine oxides, alcohols and polyglycolethers, sodium hydroxynaphthoate, sodium hydroxynaphthalene sulfonate, sodium ethylhexyl sulfate, and combinations thereof.

### Builders and Co-Builders

The detergent composition may contain about 0-65% by weight, such as about 5% to about 50% of a detergent builder or co-builder, or a mixture thereof. In a dish wash detergent, the level of builder is typically 40-65%, particularly 50-65%. The builder and/or co-builder may particularly be a chelating agent that forms water-soluble complexes with Ca and Mg. Any builder and/or co-builder known in the art for use in detergents may be utilized. Non-limiting examples of builders include zeolites, diphosphates (pyrophosphates), triphosphates such as sodium triphosphate (STP or STPP), carbonates such as sodium carbonate, soluble silicates such as sodium metasilicate, layered silicates (e.g., SKS-6 from Hoechst), ethanolamines such as 2-aminoethan-1-ol (MEA), diethanolamine (DEA, also known as 2,2′-iminodiethan-1-ol), triethanolamine (TEA, also known as 2,2′,2″-nitrilotriethan-1-ol), and (carboxymethyl)inulin (CMI), and combinations thereof.

The detergent composition may also contain 0-50% by weight, such as about 5% to about 30%, of a detergent co-builder. The detergent composition may include a co-builder alone, or in combination with a builder, for example a zeolite builder. Non-limiting examples of co-builders include homopolymers of polyacrylates or copolymers thereof, such as poly(acrylic acid) (PAA) or copoly(acrylic acid/maleic acid) (PAA/PMA). Further non-limiting examples include citrate, chelators such as aminocarboxylates, aminopolycarboxylates and phosphonates, and alkyl- or alkenylsuccinic acid.

Additional specific examples include 2,2′,2″-nitrilotriacetic acid (NTA), ethylenediaminetetraacetic acid (EDTA), diethylenetriaminepentaacetic acid (DTPA), iminodisuccinic acid (IDS), ethylenediamine-N,N-disuccinic acid (EDDS), methylglycinediacetic acid (MGDA), glutamic acid-N,N-diacetic acid (GLDA), 1-hydroxyethane-1,1-diphosphonic acid (HEDP), ethylenediaminetetra(methylenephosphonic acid) (EDTMPA), diethylenetriaminepentakis(methylenephosphonic acid) (DTMPA or DTPMPA), N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl)-aspartic acid (SMAS), N-(2-sulfoethyl)-aspartic acid (SEAS), N-(2-sulfomethyl)-glutamic acid (SMGL), N-(2-sulfoethyl)-glutamic acid (SEGL), N-methyliminodiacetic acid (MIDA), α-alanine-N,N-diacetic acid (α-ALDA), serine-N,N-diacetic acid (SEDA), isoserine-N,N-diacetic acid (ISDA), phenylalanine-N,N-diacetic acid (PHDA), anthranilic acid-N,N-diacetic acid (ANDA), sulfanilic acid-N,N-diacetic acid (SLDA), taurine-N,N-diacetic acid (TUDA) and sulfomethyl-N,N-diacetic acid (SMDA), N-(2-hydroxyethyl)ethylenediamine-N,N′,N″-riacetic acid (HEDTA), diethanolglycine (DEG), diethylenetriamine penta(methylenephosphonic acid) (DTPMP), aminotris(methylenephosphonic acid) (ATMP), and combinations and salts thereof. Further exemplary builders and/or co-builders are described in, e.g., WO 09/102854, U.S. Pat. No. 5,977,053

### Bleaching Systems

The detergent may contain 0-30% by weight, such as about 1% to about 20%, of a bleaching system. Any bleaching system known in the art for use in detergents may be utilized. Suitable bleaching system components include bleaching catalysts, photobleaches, bleach activators, sources of hydrogen peroxide such as sodium percarbonate, sodium perborates and hydrogen peroxide-urea (1:1), preformed peracids and mixtures thereof. Suitable preformed peracids include, but are not limited to, peroxycarboxylic acids and salts, diperoxydicarboxylic acids, perimidic acids and salts, peroxymonosulfuric acids and salts, for example, Oxone®, and mixtures thereof. Non-limiting examples of bleaching systems include peroxide-based bleaching systems, which may comprise, for example, an inorganic salt, including alkali metal salts such as sodium salts of perborate (usually mono- or tetra-hydrate), percarbonate, persulfate, perphosphate, persilicate salts, in combination with a peracid-forming bleach activator. The term bleach activator is meant herein as a compound which reacts with hydrogen peroxide to form a peracid via perhydrolysis. The peracid thus formed constitutes the activated bleach. Suitable bleach activators to be used herein include those belonging to the class of esters, amides, imides or anhydrides. Suitable examples are tetraacetylethylenediamine (TAED), sodium 4-[(3,5,5-trimethylhexanoyl)oxy]benzene-1-sulfonate (ISONOBS), 4-(dodecanoyloxy)benzene-1-sulfonate (LOBS), 4-(decanoyloxy)benzene-1-sulfonate, 4-(decanoyloxy)benzoate (DOBS or DOBA), 4-(nonanoyloxy)benzene-1-sulfonate (NOBS), and/or those disclosed in WO98/17767. A particular family of bleach activators of interest was disclosed in EP624154 and particularly preferred in that family is acetyl triethyl citrate (ATC). ATC or a short chain triglyceride like triacetin has the advantage that it is environmentally friendly Furthermore acetyl triethyl citrate and triacetin have good hydrolytical stability in the product upon storage and are efficient bleach activators. Finally ATC is multifunctional, as the citrate released in the perhydrolysis reaction may function as a builder. Alternatively, the bleaching system may comprise peroxyacids of, for example, the amide, imide, or sulfone type. The bleaching system may also comprise peracids such as 6-(phthalimido)peroxyhexanoic acid (PAP). The bleaching system may also include a bleach catalyst. In some embodiments the bleach component may be an organic catalyst selected from the group consisting of organic catalysts having the following formulae:

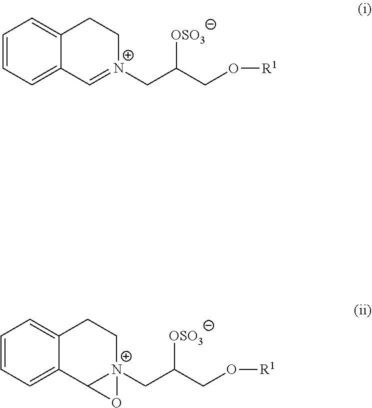

(iii) and mixtures thereof;
wherein each R¹ is independently a branched alkyl group containing from 9 to 24 carbons or linear alkyl group containing from 11 to 24 carbons, preferably each R¹ is independently a branched alkyl group containing from 9 to 18 carbons or linear alkyl group containing from 11 to 18 carbons, more preferably each R¹ is independently selected from the group consisting of 2-propylheptyl, 2-butyloctyl, 2-pentylnonyl, 2-hexyldecyl, dodecyl, tetradecyl, hexadecyl, octadecyl, isononyl, isodecyl, isotridecyl and isopentadecyl. Other exemplary bleaching systems are described, e.g. in WO2007/087258, WO2007/087244, WO2007/087259, EP1867708 (Vitamin K) and WO2007/087242. Suitable photobleaches may for example be sulfonated zinc or aluminium phthalocyanines.

Preferably the bleach component comprises a source of peracid in addition to bleach catalyst, particularly organic bleach catalyst. The source of peracid may be selected from (a) pre-formed peracid; (b) percarbonate, perborate or persulfate salt (hydrogen peroxide source) preferably in combination with a bleach activator; and (c) perhydrolase enzyme and an ester for forming peracid in situ in the presence of water in a textile or hard surface treatment step.

### Polymers

The detergent may contain 0-10% by weight, such as 0.5-5%, 2-5%, 0.5-2% or 0.2-1% of a polymer. Any polymer known in the art for use in detergents may be utilized. The polymer may function as a co-builder as mentioned above, or may provide antiredeposition, fiber protection, soil release, dye transfer inhibition, grease cleaning and/or anti-foaming properties. Some polymers may have more than one of the above-mentioned properties and/or more than one of the below-mentioned motifs. Exemplary polymers include (carboxymethyl)cellulose (CMC), poly(vinyl alcohol) (PVA), poly(vinylpyrrolidone) (PVP), poly(ethyleneglycol) or poly(ethylene oxide) (PEG), ethoxylated poly(ethyleneimine), carboxymethyl inulin (CMI), and polycarboxylates such as PAA, PAA/PMA, poly-aspartic acid, and lauryl methacrylate/acrylic acid copolymers, hydrophobically modified CMC (HM-CMC) and silicones, copolymers of terephthalic acid and oligomeric glycols, copolymers of poly(ethylene terephthalate) and poly(oxyethene terephthalate) (PET-POET), PVP, poly(vinylimidazole) (PVI), poly(vinylpyridine-N-oxide) (PVPO or PVPNO) and polyvinylpyrrolidone-vinylimidazole (PVPVI). Further exemplary polymers include sulfonated polycarboxylates, polyethylene oxide and polypropylene oxide (PEO-PPO) and diquaternium ethoxy sulfate. Other exemplary polymers are disclosed in, e.g., WO 2006/130575. Salts of the above-mentioned polymers are also contemplated.

### Soil Release Polymers

The detergent compositions of the present invention may also include one or more soil release polymers which aid the removal of soils from fabrics such as cotton and polyester based fabrics, in particular the removal of hydrophobic soils from polyester based fabrics. The soil release polymers may for example be nonionic or anionic terephthalte based polymers, polyvinyl caprolactam and related copolymers, vinyl graft copolymers, polyester polyamides see for example Chapter 7 in Powdered Detergents, Surfactant science series volume 71, Marcel Dekker, Inc. Another type of soil release polymers is amphiphilic alkoxylated grease cleaning polymers comprising a core structure and a plurality of alkoxylate groups attached to that core structure. The core structure may comprise a polyalkylenimine structure or a polyalkanolamine structure as described in detail in WO2009/087523 (hereby incorporated by reference). Furthermore, random graft co-polymers are suitable soil release polymers. Suitable graft co-polymers are described in more detail in WO2007/138054, WO2006/108856 and WO2006/113314 (hereby incorporated by reference). Suitable polyethylene glycol polymers include random graft co-polymers comprising: (i) hydrophilic backbone comprising polyethylene glycol; and (ii) side chain(s) selected from the group consisting of: C4-C25 alkyl group, polypropylene, polybutylene, vinyl ester of a saturated C1-C6 mono-carboxylic acid, CI-C 6 alkyl ester of acrylic or methacrylic acid, and mixtures thereof. Suitable polyethylene glycol polymers have a polyethylene glycol backbone with random grafted polyvinyl acetate side chains. The average molecular weight of the polyethylene glycol backbone can be in the range of from 2,000 Da to 20,000 Da, or from 4,000 Da to 8,000 Da. The molecular weight ratio of the polyethylene glycol backbone to the polyvinyl acetate side chains can be in the range of from 1:1 to 1:5, or from 1:1.2 to 1:2. The average number of graft sites per ethylene oxide units can be less than 1, or less than 0.8, the average number of graft sites per ethylene oxide units can be in the range of from 0.5 to 0.9, or the average number of graft sites per ethylene oxide units can be in the range of from 0.1 to 0.5, or from 0.2 to 0.4. A suitable polyethylene glycol polymer is Sokalan HP22. Other soil release polymers are substituted polysaccharide structures especially substituted cellulosic structures such as modified cellulose deriviatives such as those described in EP 1867808 or WO 2003/040279 (both are hereby incorporated by reference). Suitable cellulosic polymers include cellulose, cellulose ethers, cellulose esters, cellulose amides and mixtures thereof. Suitable cellulosic polymers include anionically modified cellulose, nonionically modified cellulose, cationically modified cellulose, zwitterionically modified cellulose, and mixtures thereof. Suitable cellulosic polymers include methyl cellulose, carboxy methyl cellulose, ethyl cellulose, hydroxyl ethyl cellulose, hydroxyl propyl methyl cellulose, ester carboxy methyl cellulose, and mixtures thereof.

### Dispersants

The detergent compositions of the present invention can also contain dispersants. In particular, powdered detergents may comprise dispersants. Suitable water-soluble organic materials include the homo- or co-polymeric acids or their salts, in which the polycarboxylic acid comprises at least two carboxyl radicals separated from each other by not more than two carbon atoms. Suitable dispersants are for example described in Powdered Detergents, Surfactant science series volume 71, Marcel Dekker, Inc.

### Dye Transfer Inhibiting Agents

The detergent compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in a subject composition, the dye transfer inhibiting agents may be present at levels from about 0.0001% to about 10%, from about 0.01% to about 5% or even from about 0.1% to about 3% by weight of the composition.

### Fabric Hueing Agents

The detergent compositions of the present invention may also include fabric hueing agents such as dyes or pigments, which when formulated in detergent compositions can deposit onto a fabric when said fabric is contacted with a wash liquor comprising said detergent compositions and thus altering the tint of said fabric through absorption/reflection of visible light. Fluorescent whitening agents emit at least some visible light. In contrast, fabric hueing agents alter the tint of a surface as they absorb at least a portion of the visible light spectrum. Suitable fabric hueing agents include dyes and dye-clay conjugates, and may also include pigments. Suitable dyes include small molecule dyes and polymeric dyes. Suitable small molecule dyes include small molecule dyes selected from the group consisting of dyes falling into the Colour Index (C.I.) classifications of Direct Blue, Direct Red, Direct Violet, Acid Blue, Acid Red, Acid Violet, Basic Blue, Basic Violet and Basic Red, or mixtures thereof, for example as described in WO2005/03274, WO2005/03275, WO2005/03276 and EP1876226 (hereby incorporated by reference). The detergent composition preferably comprises from about 0.00003 wt % to about 0.2 wt %, from about 0.00008 wt % to about 0.05 wt %, or even from about 0.0001 wt % to about 0.04 wt % fabric hueing agent. The composition may comprise from 0.0001 wt % to 0.2 wt % fabric hueing agent, this may be especially preferred when the composition is in the form of a unit dose pouch. Suitable hueing agents are also disclosed in, e.g. WO 2007/087257 and WO2007/087243.

### Fluorescent Whitening Agent

The detergent compositions of the present invention will preferably also contain additional components that may tint articles being cleaned, such as fluorescent whitening agent or optical brighteners. Where present the brightener is preferably at a level of about 0.01% to about 0.5%. Any fluorescent whitening agent suitable for use in a laundry detergent composition may be used in the composition of the present invention. The most commonly used fluorescent whitening agents are those belonging to the classes of diaminostilbene-sulfonic acid derivatives, diarylpyrazoline derivatives and bisphenyl-distyryl derivatives. Examples of the diaminostilbene-sulfonic acid derivative type of fluorescent whitening agents include the sodium salts of: 4,4′-bis-(2-diethanolamino-4-anilino-s-triazin-6-ylamino) stilbene-2,2′-disulfonate, 4,4′-bis-(2,4-dianilino-s-triazin-6-ylamino) stilbene-2.2′-disulfonate, 4,4′-bis-(2-anilino-4-(N-methyl-N-2-hydroxy-ethylamino)-s-triazin-6-ylamino) stilbene-2,2′-disulfonate, 4,4′-bis-(4-phenyl-1,2,3-triazol-2-yl)stilbene-2,2′-disulfonate and sodium 5-(2H-naphtho[1,2-d][1,2,3]triazol-2-yl)-2-[(E)-2-phenylvinyl]benzenesulfonate. Preferred fluorescent whitening agents are Tinopal DMS and Tinopal CBS available from Ciba-Geigy AG, Basel, Switzerland. Tinopal DMS is the disodium salt of 4,4′-bis-(2-morpholino-4-anilino-s-triazin-6-ylamino) stilbene-2,2′-disulfonate. Tinopal CBS is the disodium salt of 2,2′-bis-(phenyl-styryl)-disulfonate. Also preferred are fluorescent whitening agents is the commercially available Parawhite KX, supplied by Paramount Minerals and Chemicals, Mumbai, India. Other fluorescers suitable for use in the invention include the 1-3-diaryl pyrazolines and the 7-alkylaminocoumarins. Suitable fluorescent brightener levels include lower levels of from about 0.01, from 0.05, from about 0.1 or even from about 0.2 wt % to upper levels of 0.5 or even 0.75 wt %.

### Anti-Redeposition Agents

The detergent compositions of the present invention may also include one or more anti-redeposition agents such as carboxymethylcellulose (CMC), polyvinyl alcohol (PVA), polyvinylpyrrolidone (PVP), polyoxyethylene and/or polyethyleneglycol (PEG), homopolymers of acrylic acid, copolymers of acrylic acid and maleic acid, and ethoxylated polyethyleneimines. The cellulose based polymers described under soil release polymers above may also function as anti-redeposition agents.

### Rheology Modifiers

The detergent compositions of the present invention may also include one or more rheology modifiers, structurants or thickeners, as distinct from viscosity reducing agents. The rheology modifiers are selected from the group consisting of non-polymeric crystalline, hydroxy-functional materials, polymeric rheology modifiers which impart shear thinning characteristics to the aqueous liquid matrix of a liquid detergent composition. The rheology and viscosity of the detergent can be modified and adjusted by methods known in the art, for example as shown in EP 2169040. Other suitable cleaning composition components include, but are not limited to, anti-shrink agents, anti-wrinkling agents, bactericides, binders, carriers, dyes, enzyme stabilizers, fabric softeners, fillers, foam regulators, hydrotropes, perfumes, pigments, sod suppressors, solvents, and structurants for liquid detergents and/or structure elasticizing agents.

### Additional Enzymes

The detergent additive as well as the detergent composition may comprise one or more additional enzymes such as a protease, lipase, cutinase, an amylase, carbohydrase, cellulase, pectinase, beta-mannanase, arabinase, galactanase, xylanase, oxidase, e.g., a laccase, and/or peroxidase. In an embodiment, the additional enzyme is a beta-mannanase.

In general the properties of the selected enzyme(s) should be compatible with the selected detergent, (i.e., pH-optimum, compatibility with other enzymatic and non-enzymatic ingredients, etc.), and the enzyme(s) should be present in effective amounts.

### Cellulases

Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophila and Fusarium oxysporum disclosed in U.S. Pat. Nos. 4,435,307, 5,648,263, 5,691,178, 5,776,757 and WO 89/09259.

Especially suitable cellulases are the alkaline or neutral cellulases having colour care benefits. Examples of such cellulases are cellulases described in EP 0 495 257, EP 0 531 372, WO 96/11262, WO 96/29397, WO 98/08940. Other examples are cellulase variants such as those described in WO 94/07998, EP 0 531 315, U.S. Pat. Nos. 5,457,046, 5,686,593, 5,763,254, WO 95/24471, WO 98/12307 and WO99/001544.

Other cellulases are endo-beta-1,4-glucanase enzyme having a sequence of at least 97% identity to the amino acid sequence of position 1 to position 773 of SEQ ID NO:2 of WO 2002/099091 or a family 44 xyloglucanase, which a xyloglucanase enzyme having a sequence of at least 60% identity to positions 40-559 of SEQ ID NO: 2 of WO 2001/062903.

Commercially available cellulases include Celluzyme™, and Carezyme™ (Novozymes A/S) Carezyme Premium™ (Novozymes A/S), Celluclean™ (Novozymes A/S), Celluclean Classic™ (Novozymes A/S), Cellusoft™ (Novozymes A/S), Whitezyme™ (Novozymes A/S), Clazinase™, and Puradax HA™ (Genencor International Inc.), and KAC-500(B)™ (Kao Corporation).

### Beta-Mannanases

Suitable beta-mannanases include those of bacterial or fungal origin. Chemically or genetically modified mutants are included. The mannanase may be an alkaline beta-mannanase of Family 5 or 26. It may be a wild-type from Bacillus or Humicola, particularly B. agaradhaerens, B. licheniformis, B. halodurans, B. clausii, or H. insolens. Suitable GH5 beta-mannanases are described in WO 1999/064619, WO 2014/088940, WO 2014/100018, WO 2016/007929. Suitable GH26 beta-mannanases are described in WO 2012/149317, WO 2012/149325, WO 2012/149333, WO 2015/144782, WO 2016/054176, WO 2017/021514, WO 2017/021515, WO 2017/021516, WO 2017/021517, WO 2017/021518. A commercially available beta-mannanase is Mannaway (Novozymes A/S).

### Peroxidases/Oxidases

Suitable peroxidases/oxidases include those of plant, bacterial or fungal origin. Chemically modified or protein engineered mutants are included. Examples of useful peroxidases include peroxidases from Coprinus, e.g., from C. cinereus, and variants thereof as those described in WO 93/24618, WO 95/10602, and WO 98/15257. Commercially available peroxidases include Guardzyme™ (Novozymes A/S).

### Proteases

Suitable proteases include those of bacterial, fungal, plant, viral or animal origin e.g. vegetable or microbial origin. Microbial origin is preferred. Chemically modified or protein engineered mutants are included. It may be an alkaline protease, such as a serine protease or a metalloprotease. A serine protease may for example be of the S1 family, such as trypsin, or the S8 family such as subtilisin. A metalloproteases protease may for example be a thermolysin from e.g. family M4 or other metalloprotease such as those from M5, M7 or M8 families.

The term “subtilases” refers to a sub-group of serine protease according to Siezen et al., Protein Engng. 4 (1991) 719-737 and Siezen et al. Protein Science 6 (1997) 501-523. Serine proteases are a subgroup of proteases characterized by having a serine in the active site, which forms a covalent adduct with the substrate. The subtilases may be divided into 6 sub-divisions, i.e. the Subtilisin family, the Thermitase family, the Proteinase K family, the Lantibiotic peptidase family, the Kexin family and the Pyrolysin family.

Examples of subtilases are those derived from Bacillus such as Bacillus lentus, Bacillus alkalophilus, Bacillus subtilis, Bacillus amyloliquefaciens, Bacillus pumilus and Bacillus gibsonii described in; U.S. Pat. No. 7,262,042 and WO09/021867, and Subtilisin lentus, Subtilisin Novo, subtilisin Carlsberg, Bacillus licheniformis, subtilisin BPN′, subtilisin 309, subtilisin 147 and subtilisin 168 and e.g. protease PD138 described in (WO93/18140). Other useful proteases may be those described in WO01/016285 and WO02/016547. Examples of trypsin-like proteases are trypsin (e.g. of porcine or bovine origin) and the Fusarium protease described in WO94/25583 and WO05/040372, and the chymotrypsin proteases derived from Cellumonas described in WO05/052161 and WO05/052146.

A further preferred protease is the alkaline protease from Bacillus lentus DSM 5483, as described for example in WO95/23221, and variants thereof which are described in WO92/21760, WO95/23221, EP1921147 and EP1921148.

Examples of metalloproteases are the neutral metalloprotease as described in WO07/044993 (Proctor & Gamble/Genencor Int.) such as those derived from Bacillus amyloliquefaciens.

Examples of useful proteases are the variants described in: WO89/06279 WO92/19729, WO96/034946, WO98/20115, WO98/20116, WO99/011768, WO01/44452, WO03/006602, WO04/03186, WO04/041979, WO07/006305, WO11/036263, WO11/036264, especially the variants with substitutions in one or more of the following positions: 3, 4, 9, 15, 24, 27, 42, 55, 59, 60, 66, 74, 85, 96, 97, 98, 99, 100, 101, 102, 104, 116, 118, 121, 126, 127, 128, 154, 156, 157, 158, 161, 164, 176, 179, 182, 185, 188, 189, 193, 198, 199, 200, 203, 206, 211, 212, 216, 218, 226, 229, 230, 239, 246, 255, 256, 268 and 269 wherein the positions correspond to the positions of the Bacillus lentus protease shown in SEQ ID NO 1 of WO 2016/001449. More preferred the protease variants may comprise one or more of the mutations selected from the group consisting of: S3T, V41, S9R, S9E, A15T, S24G, S24R, K27R, N42R, S55P, G59E, G59D, N60D, N60E, V66A, N74D, S85R, A96S, S97G, S97D, S97A, S97SD, S99E, S99D, S99G, S99M, S99N, S99R, S99H, S101A, V102I, V102Y, V102N, S104A, G116V, G116R, H118D, H118N, A120S, S126L, P127Q, S128A, S154D, A156E, G157D, G157P, S158E, Y161A, R164S, Q176E, N179E, S182E, Q185N, A188P, G189E, V193M, N198D, V199I, Y203W, S206G, L211Q, L211D, N212D, N212S, M216S, A226V, K229L, Q230H, Q239R, N246K, N255W, N255D, N255E, L256E, L256D T268A and R269H. The protease variants are preferably variants of the Bacillus lentus protease shown in SEQ ID NO 1 of WO2016/001449, the Bacillus amylolichenifaciens protease (BPN′) shown in SEQ ID NO 2 of WO2016/001449. The protease variants preferably have at least 80% sequence identity to SEQ ID NO 1 or SEQ ID NO 2 of WO 2016/001449.

A protease variant comprising a substitution at one or more positions corresponding to positions 171, 173, 175, 179, or 180 of SEQ ID NO: 1 of WO2004/067737, wherein said protease variant has a sequence identity of at least 75% but less than 100% to SEQ ID NO: 1 of WO2004/067737.

Suitable commercially available protease enzymes include those sold under the trade names Alcalase®, Duralase™, Durazym™, Relase®, Relase® Ultra, Savinase®, Savinase® Ultra, Primase®, Polarzyme®, Kannase®, Liquanase®, Liquanase® Ultra, Ovozyme®, Coronase®, Coronase® Ultra, Blaze®, Blaze Evity® 100T, Blaze Evity® 125T, Blaze Evity® 150T, Neutrase®, Everlase® and Esperase® (Novozymes A/S), those sold under the tradename Maxatase®, Maxacal®, Maxapem®, Purafect Ox®, Purafect OxP®, Puramax®, FN2®, FN3®, FN4®, Excellase®, Excellenz P1000™, Excellenz P1250™, Eraser®, Preferenz P100™, Purafect Prime®, Preferenz P110™, Effectenz P1000™, Purafect®™, Effectenz P1050™, Purafect Ox®™, Effectenz P2000™, Purafast®, Properase®, Opticlean® and Optimase® (Danisco/DuPont), Axapem™ (Gist-Brocases N.V.), BLAP (sequence shown in FIG. 29 of U.S. Pat. No. 5,352,604) and variants hereof (Henkel AG) and KAP (Bacillus alkalophilus subtilisin) from Kao.

### Lipases and Cutinases:

Suitable lipases and cutinases include those of bacterial or fungal origin. Chemically modified or protein engineered mutant enzymes are included. Examples include lipase from Thermomyces, e.g. from T. lanuginosus (previously named Humicola lanuginosa) as described in EP258068 and EP305216, cutinase from Humicola, e.g. H. insolens (WO96/13580), lipase from strains of Pseudomonas (some of these now renamed to Burkholderia), e.g. P. alcaligenes or P. pseudoalcaligenes (EP218272), P. cepacia (EP331376), P. sp. strain SD705 (WO95/06720 & WO96/27002), P. wisconsinensis (WO96/12012), GDSL-type Streptomyces lipases (WO10/065455), cutinase from Magnaporthe grisea (WO10/107560), cutinase from Pseudomonas mendocina (U.S. Pat. No. 5,389,536), lipase from Thermobifida fusca (WO11/084412), Geobacillus stearothermophilus lipase (WO11/084417), lipase from Bacillus subtilis (WO11/084599), and lipase from Streptomyces griseus (WO 11/150157) and S. pristinaespiralis (WO12/137147).

Other examples are lipase variants such as those described in EP407225, WO92/05249, WO94/01541, WO94/25578, WO95/14783, WO95/30744, WO95/35381, WO95/22615, WO96/00292, WO97/04079, WO97/07202, WO00/34450, WO00/60063, WO01/92502, WO07/87508 and WO09/109500.

Preferred commercial lipase products include Lipolase™, Lipex™; Lipolex™ and Lipoclean™ (Novozymes A/S), Lumafast (originally from Genencor) and Lipomax (originally from Gist-Brocades).

Still other examples are lipases sometimes referred to as acyltransferases or perhydrolases, e.g. acyltransferases with homology to Candida antarctica lipase A (WO10/111143), acyltransferase from Mycobacterium smegmatis (WO05/56782), perhydrolases from the CE 7 family (WO09/67279), and variants of the M. smegmatis perhydrolase in particular the S54V variant used in the commercial product Gentle Power Bleach from Huntsman Textile Effects Pte Ltd (WO10/100028).

### Amylases:

Suitable amylases which can be used together with alpha-mannan degrading enzymes may be an alpha-amylase or a glucoamylase and may be of bacterial or fungal origin. Chemically modified or protein engineered mutants are included. Amylases include, for example, alpha-amylases obtained from Bacillus, e.g., a special strain of Bacillus licheniformis, described in more detail in GB 1,296,839.

Suitable amylases include amylases having SEQ ID NO: 2 in WO 95/10603 or variants having 90% sequence identity to SEQ ID NO: 3 thereof. Preferred variants are described in WO 94/02597, WO 94/18314, WO 97/43424 and SEQ ID NO: 4 of WO 99/019467, such as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391, 408, and 444.

Different suitable amylases include amylases having SEQ ID NO: 6 in WO 02/010355 or variants thereof having 90% sequence identity to SEQ ID NO: 6. Preferred variants of SEQ ID NO: 6 are those having a deletion in positions 181 and 182 and a substitution in position 193.

Other amylases which are suitable are hybrid alpha-amylase comprising residues 1-33 of the alpha-amylase derived from B. amyloliquefaciens shown in SEQ ID NO: 6 of WO 2006/066594 and residues 36-483 of the B. licheniformis alpha-amylase shown in SEQ ID NO: 4 of WO 2006/066594 or variants having 90% sequence identity thereof. Preferred variants of this hybrid alpha-amylase are those having a substitution, a deletion or an insertion in one of more of the following positions: G48, T49, G107, H156, A181, N190, M197, I201, A209 and Q264. Most preferred variants of the hybrid alpha-amylase comprising residues 1-33 of the alpha-amylase derived from B. amyloliquefaciens shown in SEQ ID NO: 6 of WO 2006/066594 and residues 36-483 of SEQ ID NO: 4 are those having the substitutions:

$$\begin{matrix}M 197 T ; \\ H 156 Y + A 181 T + N 190 F + A 209 V + Q 264 S ; or \\ G 48 A + T 491 + G 107 A + H 156 Y + A 181 T + N 190 F + 1201 F + A 209 V + Q 264 S .\end{matrix}$$

Further amylases which are suitable are amylases having SEQ ID NO: 6 in WO 99/019467 or variants thereof having 90% sequence identity to SEQ ID NO: 6. Preferred variants of SEQ ID NO: 6 are those having a substitution, a deletion or an insertion in one or more of the following positions: R181, G182, H183, G184, N195, I206, E212, E216 and K269. Particularly preferred amylases are those having deletion in positions R181 and G182, or positions H183 and G184.

Additional amylases which can be used are those having SEQ ID NO: 1, SEQ ID NO: 3, SEQ ID NO: 2 or SEQ ID NO: 7 of WO 96/023873 or variants thereof having 90% sequence identity to SEQ ID NO: 1, SEQ ID NO: 2, SEQ ID NO: 3 or SEQ ID NO: 7. Preferred variants of SEQ ID NO: 1, SEQ ID NO: 2, SEQ ID NO: 3 or SEQ ID NO: 7 are those having a substitution, a deletion or an insertion in one or more of the following positions: 140, 181, 182, 183, 184, 195, 206, 212, 243, 260, 269, 304 and 476, using SEQ ID 2 of WO 96/023873 for numbering. More preferred variants are those having a deletion in two positions selected from 181, 182, 183 and 184, such as 181 and 182, 182 and 183, or positions 183 and 184. Most preferred amylase variants of SEQ ID NO: 1, SEQ ID NO: 2 or SEQ ID NO: 7 are those having a deletion in positions 183 and 184 and a substitution in one or more of positions 140, 195, 206, 243, 260, 304 and 476.

Other amylases which can be used are amylases having SEQ ID NO: 2 of WO 08/153815, SEQ ID NO: 10 in WO 01/66712 or variants thereof having 90% sequence identity to SEQ ID NO: 2 of WO 08/153815 or 90% sequence identity to SEQ ID NO: 10 in WO 01/66712. Preferred variants of SEQ ID NO: 10 in WO 01/66712 are those having a substitution, a deletion or an insertion in one of more of the following positions: 176, 177, 178, 179, 190, 201, 207, 211 and 264.

Further suitable amylases are amylases having SEQ ID NO: 2 of WO 09/061380 or variants having 90% sequence identity to SEQ ID NO: 2 thereof. Preferred variants of SEQ ID NO: 2 are those having a truncation of the C-terminus and/or a substitution, a deletion or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO: 2 are those having the substitution in one of more of the following positions: Q87E,R, Q98R, S125A, N128C, T131I, T165I, K178L, T182G, M201L, F202Y, N225E,R, N272E,R, S243Q,A,E,D, Y305R, R309A, Q320R, Q359E, K444E and G475K and/or deletion in position R180 and/or S181 or of T182 and/or G183. Most preferred amylase variants of SEQ ID NO: 2 are those having the substitutions:

$$\begin{matrix}N 128 C + K 178 L + T 182 G + Y 305 R + G 4 \; 75 K ; \\ N 128 C + K 178 L + T 182 G + F 202 Y + Y 305 R + D 319 T + G 4 \; 75 K ; \\ S 125 A + N 128 C + K 178 L + T 182 G + Y 305 R + G 4 \; 75 K ; or\end{matrix}$$

S125A+N128C+T131I+T165I+K178L+T182G+Y305R+G475K wherein the variants are C-terminally truncated and optionally further comprises a substitution at position 243 and/or a deletion at position 180 and/or position 181.

Further suitable amylases are amylases having SEQ ID NO: 1 of WO13184577 or variants having 90% sequence identity to SEQ ID NO: 1 thereof. Preferred variants of SEQ ID NO: 1 are those having a substitution, a deletion or an insertion in one of more of the following positions: K176, R178, G179, T180, G181, E187, N192, M199, I203, S241, R458, T459, D460, G476 and G477. More preferred variants of SEQ ID NO: 1 are those having the substitution in one of more of the following positions: K176L, E187P, N192FYH, M199L, 1203YF, S241 QADN, R458N, T459S, D460T, G476K and G477K and/or deletion in position R178 and/or S179 or of T180 and/or G181. Most preferred amylase variants of SEQ ID NO: 1 are those having the substitutions:

$$\begin{matrix}E 187 P + I 203 Y + G 476 K \\ E 187 P + I 203 Y + R 458 N + T 459 S + D 460 T + G 476 K\end{matrix}$$

wherein the variants optionally further comprise a substitution at position 241 and/or a deletion at position 178 and/or position 179.

Further suitable amylases are amylases having SEQ ID NO: 1 of WO10104675 or variants having 90% sequence identity to SEQ ID NO: 1 thereof. Preferred variants of SEQ ID NO: 1 are those having a substitution, a deletion or an insertion in one of more of the following positions: N21, D97, V128 K177, R179, S180, I181, G182, M200, L204, E242, G477 and G478. More preferred variants of SEQ ID NO: 1 are those having the substitution in one of more of the following positions: N21 D, D97N, V128I K177L, M200L, L204YF, E242QA, G477K and G478K and/or deletion in position R179 and/or S180 or of 1181 and/or G182. Most preferred amylase variants of SEQ ID NO: 1 are those having the substitutions:

$$N 21 D + D 97 N + V 128 I$$

wherein the variants optionally further comprise a substitution at position 200 and/or a deletion at position 180 and/or position 181.

Other suitable amylases are the alpha-amylase having SEQ ID NO: 12 in WO01/66712 or a variant having at least 90% sequence identity to SEQ ID NO: 12. Preferred amylase variants are those having a substitution, a deletion or an insertion in one of more of the following positions of SEQ ID NO: 12 in WO01/66712: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484. Particular preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions.

Other examples are amylase variants such as those described in WO2011/098531, WO2013/001078 and WO2013/001087.

Commercially available amylases are Duramyl™, Termamyl™, Fungamyl™, Stainzyme™ Stainzyme Plus™, Natalase™, Liquozyme X and BAN™ (from Novozymes A/S), and Rapidase™ Purastar™/Effectenz™, Powerase, Preferenz S1000, Preferenz S100 and Preferenz S110 (from Genencor International Inc./DuPont).

### Peroxidases/Oxidases

A peroxidase according to the invention is a peroxidase enzyme comprised by the enzyme classification EC 1.11.1.7, as set out by the Nomenclature Committee of the International Union of Biochemistry and Molecular Biology (IUBMB), or any fragment derived therefrom, exhibiting peroxidase activity.

Suitable peroxidases include those of plant, bacterial or fungal origin. Chemically modified or protein engineered mutants are included. Examples of useful peroxidases include peroxidases from Coprinopsis, e.g., from C. cinerea (EP 179,486), and variants thereof as those described in WO 93/24618, WO 95/10602, and WO 98/15257.

A peroxidase according to the invention also include a haloperoxidase enzyme, such as chloroperoxidase, bromoperoxidase and compounds exhibiting chloroperoxidase or bromoperoxidase activity. Haloperoxidases are classified according to their specificity for halide ions. Chloroperoxidases (E.C. 1.11.1.10) catalyze formation of hypochlorite from chloride ions.

In an embodiment, the haloperoxidase of the invention is a chloroperoxidase. Preferably, the haloperoxidase is a vanadium haloperoxidase, i.e., a vanadate-containing haloperoxidase. In a preferred method of the present invention the vanadate-containing haloperoxidase is combined with a source of chloride ion.

Haloperoxidases have been isolated from many different fungi, in particular from the fungus group dematiaceous hyphomycetes, such as Caldariomyces, e.g., C. fumago, Alternaria, Curvularia, e.g., C. verruculosa and C. inaequalis, Drechslera, Ulocladium and Botrytis.

Haloperoxidases have also been isolated from bacteria such as Pseudomonas, e.g., P. pyrrocinia and Streptomyces, e.g., S. aureofaciens.

In an preferred embodiment, the haloperoxidase is derivable from Curvularia sp., in particular Curvularia verruculosa or Curvularia inaequalis, such as C. inaequalis CBS 102.42 as described in WO 95/27046; or C. verruculosa CBS 147.63 or C. verruculosa CBS 444.70 as described in WO 97/04102; or from Drechslera hartlebii as described in WO 01/79459, Dendryphiella salina as described in WO 01/79458, Phaeotrichoconis crotalarie as described in WO 01/79461, or Geniculosporium sp. as described in WO 01/79460.

An oxidase according to the invention include, in particular, any laccase enzyme comprised by the enzyme classification EC 1.10.3.2, or any fragment derived therefrom exhibiting laccase activity, or a compound exhibiting a similar activity, such as a catechol oxidase (EC 1.10.3.1), an o-aminophenol oxidase (EC 1.10.3.4), or a bilirubin oxidase (EC 1.3.3.5).

Preferred laccase enzymes are enzymes of microbial origin. The enzymes may be derived from plants, bacteria or fungi (including filamentous fungi and yeasts).

Suitable examples from fungi include a laccase derivable from a strain of Aspergillus, Neurospora, e.g., N. crassa, Podospora, Botrytis, Collybia, Fomes, Lentinus, Pleurotus, Trametes, e.g., T. villosa and T. versicolor, Rhizoctonia, e.g., R. solani, Coprinopsis, e.g., C. cinerea, C. comatus, C. friesii, and C. plicatilis, Psathyrella, e.g., P. condelleana, Panaeolus, e.g., P. papilionaceus, Myceliophthora, e.g., M. thermophila, Schytalidium, e.g., S. thermophilum, Polyporus, e.g., P. pinsitus, Phlebia, e.g., P. radiata (WO 92/01046), or Coriolus, e.g., C. hirsutus (JP 2238885).

Suitable examples from bacteria include a laccase derivable from a strain of Bacillus.

A laccase derived from Coprinopsis or Myceliophthora is preferred; in particular a laccase derived from Coprinopsis cinerea, as disclosed in WO 97/08325; or from Myceliophthora thermophila, as disclosed in WO 95/33836.

The detergent enzyme(s) may be included in a detergent composition by adding separate additives containing one or more enzymes, or by adding a combined additive comprising all of these enzymes. A detergent additive of the invention, i.e., a separate additive or a combined additive, can be formulated, for example, as a granulate, liquid, slurry, etc. Preferred detergent additive formulations are granulates, in particular non-dusting granulates, liquids, in particular stabilized liquids, or slurries.

Non-dusting granulates may be produced, e.g. as disclosed in U.S. Pat. Nos. 4,106,991 and 4,661,452 and may optionally be coated by methods known in the art. Examples of waxy coating materials are polyethyleneglycol (PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591. Liquid enzyme preparations may, for instance, be stabilized by adding a polyol such as propylene glycol, a sugar or sugar alcohol, lactic acid or boric acid according to established methods. Protected enzymes may be prepared according to the method disclosed in EP 238,216.

### Microorganisms

The detergent additive as well as the detergent composition may also comprise one or more microorganisms, such as one or more fungi, yeast, or bacteria.

In an embodiment, the one or more microorganisms are dehydrated (for example by lyophilization) bacteria or yeast, such as a strain of Lactobacillus.

In another embodiment, the microrganisms are one or more microbial spores (as opposed to vegetative cells), such as bacterial spores; or fungal spores, conidia, hypha. Preferably, the one or more spores are Bacillus endospores; even more preferably the one or more spores are endospores of Bacillus subtilis, Bacillus licheniformis, Bacillus amyloliquefaciens, or Bacillus megaterium.

The microrganisms may be included in the detergent composition or additive in the same way as enzymes (see above).

### Adjunct Materials

Other suitable adjunct materials include, but are not limited to, anti-shrink agents, anti-wrinkling agents, bactericides, binders, carriers, dyes, enzyme stabilizers, fabric softeners, fillers, foam regulators, hydrotropes, perfumes, pigments, sod suppressors, solvents, structurants for liquid detergents and/or structure elasticizing agents.

In one aspect the detergent is a compact fluid laundry detergent composition comprising: a) at least about 10%, preferably from 20 to 80% by weight of the composition, of surfactant selected from anionic surfactants, non ionic surfactants, soap and mixtures thereof; b) from about 1% to about 30%, preferably from 5 to 30%, by weight of the composition, of water; c) from about 1% to about 15%, preferably from 3 to 10% by weight of the composition, of non-aminofunctional solvent; and d) from about 5% to about 20%, by weight of the composition, of a performance additive selected from chelants, soil release polymers, enzymes and mixtures thereof; wherein the compact fluid laundry detergent composition comprises at least one of: (i) the surfactant has a weight ratio of the anionic surfactant to the nonionic surfactant from about 1.5:1 to about 5:1, the surfactant comprises from about 15% to about 40%, by weight of the composition, of anionic surfactant and comprises from about 5% to about 40%, by weight of the composition, of the soap; (ii) from about 0.1% to about 10%, by weight of the composition, of a suds boosting agent selected from suds boosting polymers, cationic surfactants, zwitterionic surfactants, amine oxide surfactants, amphoteric surfactants, and mixtures thereof; and (ii) both (i) and (ii). All the ingredients are described in WO 2007/130562. Further polymers useful in detergent formulations are described in WO 2007/149806.

In another aspect the detergent is a compact granular (powdered) detergent comprising a) at least about 10%, preferably from 15 to 60% by weight of the composition, of surfactant selected from anionic surfactants, non-ionic surfactants, soap and mixtures thereof; b) from about 10 to 80% by weight of the composition, of a builder, preferably from 20% to 60% where the builder may be a mixture of builders selected from i) phosphate builder, preferably less than 20%, more preferably less than 10% even more preferably less than 5% of the total builder is a phosphate builder; ii) a zeolite builder, preferably less than 20%, more preferably less than 10% even more preferably less than 5% of the total builder is a zeolite builder; iii) citrate, preferably 0 to 5% of the total builder is a citrate builder; iv) polycarboxylate, preferably 0 to 5% of the total builder is a polycarboxylate builder v) carbonate, preferably 0 to 30% of the total builder is a carbonate builder and vi) sodium silicates, preferably 0 to 20% of the total builder is a sodium silicate builder; c) from about 0% to 25% by weight of the composition, of fillers such as sulphate salts, preferably from 1% to 15%, more preferably from 2% to 10%, more preferably from 3% to 5% by weight of the composition, of fillers; and d) from about 0.1% to 20% by weight of the composition, of enzymes, preferably from 1% to 15%, more preferably from 2% to 10% by weight of the composition, of enzymes.

The soils and stains that are important for detergent formulators are composed of many different substances, and a range of different enzymes, all with different substrate specificities have been developed for use in detergents both in relation to laundry and hard surface cleaning, such as dishwashing. These enzymes are considered to provide an enzyme detergency benefit, since they specifically improve stain removal in the cleaning process they are applied in as compared to the same process without enzymes. Stain removing enzymes that are known in the art include enzymes such as carbohydrases, amylases, proteases, lipases, cellulases, hemicellulases, xylanases, cutinases, and pectinase.

In a preferred aspect of the present invention an alpha-mannan degrading enzyme may be combined with at least two additional enzymes. These additional enzymes are described in details in the section “other enzymes”, more preferred at least three, four or five enzymes. Preferably, the enzymes have different substrate specificity, e.g., carbolytic activity, proteolytic activity, amylolytic activity, lipolytic activity, hemicellulytic activity or pectolytic activity. The enzyme combination may for example be an alpha-mannan degrading enzyme with a stain removing enzyme, e.g., an alpha-mannan degrading enzyme and a protease, an alpha-mannan degrading enzyme and a serine protease, an alpha-mannan degrading enzyme and an amylase, an alpha-mannan degrading enzyme and a cellulase, an alpha-mannan degrading enzyme and a lipase, an alpha-mannan degrading enzyme and a cutinase, an alpha-mannan degrading enzyme and a pectinase, an alpha-mannan degrading enzyme and a beta-mannanase, or an alpha-mannan degrading enzyme and an anti-redeposition enzyme. More preferably, the an alpha-mannan degrading enzyme is combined with at least two stain removing enzymes, e.g., an alpha-mannan degrading enzyme, a lipase and an amylase; or an alpha-mannan degrading enzyme, a protease and an amylase; or an alpha-mannan degrading enzyme, a protease and a lipase; or an alpha-mannan degrading enzyme, a protease and a pectinase; or an alpha-mannan degrading enzyme, a protease and a cellulase; or an alpha-mannan degrading enzyme, a protease and a hemicellulase; or an alpha-mannan degrading enzyme, a protease and a cutinase; or an alpha-mannan degrading enzyme, an amylase and a pectinase; or an alpha-mannan degrading enzyme, an amylase and a cutinase; or an alpha-mannan degrading enzyme, an amylase and a cellulase; or an alpha-mannan degrading enzyme, an amylase and a hemicellulase; or an alpha-mannan degrading enzyme, a lipase and a pectinase; or an alpha-mannan degrading enzyme, a lipase and a cutinase; an alpha-mannan degrading enzyme, a lipase and a cellulase; or an alpha-mannan degrading enzyme, a lipase and a hemicellulase. Even more preferably, an alpha-mannan degrading enzyme may be combined with at least three stain removing enzymes, e.g., an alpha-mannan degrading enzyme, a protease, a lipase and an amylase; or an alpha-mannan degrading enzyme, a protease, an amylase and a pectinase; or an alpha-mannan degrading enzyme, a protease, an amylase and a cutinase; or an alpha-mannan degrading enzyme, a protease, an amylase and a cellulase; or an alpha-mannan degrading enzyme, a protease, an amylase and a hemicellulase; an alpha-mannan degrading enzyme, an amylase, a lipase and a pectinase; or an alpha-mannan degrading enzyme, an amylase, a lipase and a cutinase; or an alpha-mannan degrading enzyme, an amylase, a lipase and a cellulase; or an alpha-mannan degrading enzyme, an amylase, a lipase and a hemicellulase; or an alpha-mannan degrading enzyme, a protease, a lipase and a pectinase; or an alpha-mannan degrading enzyme, a protease, a lipase and a cutinase; or an alpha-mannan degrading enzyme, a protease, a lipase and a cellulase; or an alpha-mannan degrading enzyme, a protease, a lipase and a hemicellulase. An alpha-mannan degrading enzyme may be combined with any of the enzymes selected from the non-exhaustive list comprising: carbohydrases, such as an amylase, a hemicellulase, a pectinase, a cellulase, a beta-mannanase, a xanthanase or a pullulanase, a peptidase, a protease or a lipase.

In a preferred embodiment, an alpha-mannan degrading enzyme is combined with a serine protease, e.g., an S8 family protease such as Savinase®.

In another embodiment of the present invention, an alpha-mannan degrading enzyme may be combined with one or more metalloproteases, such as an M4 metalloprotease, including Neutrase® or Thermolysin. Such combinations may further comprise combinations of the other detergent enzymes as outlined above.

The cleaning process or the textile care process may for example be a laundry process, a dishwashing process or cleaning of hard surfaces such as bathroom tiles, floors, table tops, drains, sinks and washbasins. Laundry processes can for example be household laundering, but it may also be industrial laundering. Furthermore, the invention relates to a process for laundering of fabrics and/or garments where the process comprises treating fabrics with a washing solution containing a detergent composition, and at least one mannanase of the invention. The cleaning process or a textile care process can for example be carried out in a machine washing process or in a manual washing process. The washing solution can for example be an aqueous washing solution containing a detergent composition.

The fabrics and/or garments subjected to a washing, cleaning or textile care process of the present invention may be conventional washable laundry, for example household laundry. Preferably, the major part of the laundry is garments and fabrics, including knits, woven, denims, non-woven, felts, yarns, and towelling. The fabrics may be cellulose based such as natural cellulosics, including cotton, flax, linen, jute, ramie, sisal or coir or manmade cellulosics (e.g., originating from wood pulp) including viscose/rayon, ramie, cellulose acetate fibers (tricell), lyocell or blends thereof. The fabrics may also be non-cellulose based such as natural polyamides including wool, camel, cashmere, mohair, rabit and silk or synthetic polymer such as nylon, aramid, polyester, acrylic, polypropylen and spandex/elastane, or blends thereof as well as blend of cellulose based and non-cellulose based fibers. Examples of blends are blends of cotton and/or rayon/viscose with one or more companion material such as wool, synthetic fibers (e.g., polyamide fibers, acrylic fibers, polyester fibers, polyvinyl alcohol fibers, polyvinyl chloride fibers, polyurethane fibers, polyurea fibers, aramid fibers), and cellulose-containing fibers (e.g., rayon/viscose, ramie, flax, linen, jute, cellulose acetate fibers, lyocell).

The last few years there has been an increasing interest in replacing components in detergents, which is derived from petrochemicals with renewable biological components such as enzymes and polypeptides without compromising the wash performance. When the components of detergent compositions change new enzyme activities or new enzymes having alternative and/or improved properties compared to the common used detergent enzymes such as proteases, lipases and amylases is needed to achieve a similar or improved wash performance when compared to the traditional detergent compositions.

Typical detergent compositions include various components in addition to the enzymes, these components have different effects, some components like the surfactants lower the surface tension in the detergent, which allows the stain being cleaned to be lifted and dispersed and then washed away, other components like bleach systems removes discolor often by oxidation and many bleaches also have strong bactericidal properties, and are used for disinfecting and sterilizing. Yet other components like builder and chelator softens, e.g., the wash water by removing the metal ions from the liquid.

In a particular embodiment, the invention concerns the use of a composition comprising an alpha-mannan degrading enzyme, wherein said enzyme composition further comprises at least one or more of the following a surfactant, a builder, a chelator or chelating agent, bleach system or bleach component in laundry or dish wash.

In a preferred embodiment of the invention the amount of a surfactant, a builder, a chelator or chelating agent, bleach system and/or bleach component are reduced compared to amount of surfactant, builder, chelator or chelating agent, bleach system and/or bleach component used without an alpha-mannan degrading enzyme. Preferably the at least one component which is a surfactant, a builder, a chelator or chelating agent, bleach system and/or bleach component is present in an amount that is 1% less, such as 2% less, such as 3% less, such as 4% less, such as 5% less, such as 6% less, such as 7% less, such as 8% less, such as 9% less, such as 10% less, such as 15% less, such as 20% less, such as 25% less, such as 30% less, such as 35% less, such as 40% less, such as 45% less, such as 50% less than the amount of the component in the system without the addition of an alpha-mannan degrading enzyme, such as a conventional amount of such component.

In one aspect, the alpha-mannan degrading enzyme is used in detergent compositions wherein said composition is free of at least one component which is a surfactant, a builder, a chelator or chelating agent, bleach system or bleach component and/or polymer.

### Formulation of Detergent Products

The detergent composition of the invention may be in any convenient form, e.g., a bar, a homogenous tablet, a tablet having two or more layers, a pouch having one or more compartments, a regular or compact powder, a granule, a paste, a gel, or a regular, compact or concentrated liquid.

Pouches can be configured as single or multicompartments. It can be of any form, shape and material which is suitable for hold the composition, e.g. without allowing the release of the composition to release of the composition from the pouch prior to water contact. The pouch is made from water soluble film which encloses an inner volume. Said inner volume can be divided into compartments of the pouch. Preferred films are polymeric materials preferably polymers which are formed into a film or sheet. Preferred polymers, copolymers or derivates thereof are selected polyacrylates, and water soluble acrylate copolymers, methyl cellulose, carboxy methyl cellulose, sodium dextrin, ethyl cellulose, hydroxyethyl cellulose, hydroxypropyl methyl cellulose, malto dextrin, poly methacrylates, most preferably polyvinyl alcohol copolymers and, hydroxypropyl methyl cellulose (HPMC). Preferably the level of polymer in the film for example PVA is at least about 60%. Preferred average molecular weight will typically be about 20,000 to about 150,000. Films can also be of blended compositions comprising hydrolytically degradable and water soluble polymer blends such as polylactide and polyvinyl alcohol (known under the Trade reference M8630 as sold by MonoSol LLC, Indiana, USA) plus plasticisers like glycerol, ethylene glycerol, propylene glycol, sorbitol and mixtures thereof. The pouches can comprise a solid laundry cleaning composition or part components and/or a liquid cleaning composition or part components separated by the water soluble film. The compartment for liquid components can be different in composition than compartments containing solids: US2009/0011970 A1.

Detergent ingredients can be separated physically from each other by compartments in water dissolvable pouches or in different layers of tablets. Thereby negative storage interaction between components can be avoided. Different dissolution profiles of each of the compartments can also give rise to delayed dissolution of selected components in the wash solution.

A liquid or gel detergent, which is not unit dosed, may be aqueous, typically containing at least 20% by weight and up to 95% water, such as up to about 70% water, up to about 65% water, up to about 55% water, up to about 45% water, up to about 35% water. Other types of liquids, including without limitation, alkanols, amines, diols, ethers and polyols may be included in an aqueous liquid or gel. An aqueous liquid or gel detergent may contain from 0-30% organic solvent.

A liquid or gel detergent may be non-aqueous.

### Laundry Soap Bars

Alpha-mannan degrading enzymes may be added to laundry soap bars and used for hand washing laundry, fabrics and/or textiles. The term laundry soap bar includes laundry bars, soap bars, combo bars, syndet bars and detergent bars. The types of bar usually differ in the type of surfactant they contain, and the term laundry soap bar includes those containing soaps from fatty acids and/or synthetic soaps. The laundry soap bar has a physical form which is solid and not a liquid, gel or a powder at room temperature. The term solid is defined as a physical form which does not significantly change over time, i.e. if a solid object (e.g. laundry soap bar) is placed inside a container, the solid object does not change to fill the container it is placed in. The bar is a solid typically in bar form but can be in other solid shapes such as round or oval.

The laundry soap bar may contain one or more additional enzymes, protease inhibitors such as peptide aldehydes (or hydrosulfite adduct or hemiacetal adduct), boric acid, borate, borax and/or phenylboronic acid derivatives such as 4-formylphenylboronic acid, one or more soaps or synthetic surfactants, polyols such as glycerine, pH controlling compounds such as fatty acids, citric acid, acetic acid and/or formic acid, and/or a salt of a monovalent cation and an organic anion wherein the monovalent cation may be for example Na⁺, K⁺ or NH₄⁺ and the organic anion may be for example formate, acetate, citrate or lactate such that the salt of a monovalent cation and an organic anion may be, for example, sodium formate.

The laundry soap bar may also contain complexing agents like EDTA and HEDP, perfumes and/or different type of fillers, surfactants e.g. anionic synthetic surfactants, builders, polymeric soil release agents, detergent chelators, stabilizing agents, fillers, dyes, colorants, dye transfer inhibitors, alkoxylated polycarbonates, suds suppressers, structurants, binders, leaching agents, bleaching activators, clay soil removal agents, anti-redeposition agents, polymeric dispersing agents, brighteners, fabric softeners, perfumes and/or other compounds known in the art.

The laundry soap bar may be processed in conventional laundry soap bar making equipment such as but not limited to: mixers, plodders, e.g a two stage vacuum plodder, extruders, cutters, logo-stampers, cooling tunnels and wrappers. The invention is not limited to preparing the laundry soap bars by any single method. The premix of the invention may be added to the soap at different stages of the process. For example, the premix containing a soap, alpha-mannan degrading enzyme, optionally one or more additional enzymes, a protease inhibitor, and a salt of a monovalent cation and an organic anion may be prepared and the mixture is then plodded. The alpha-mannan degrading enzyme and optional additional enzymes may be added at the same time as the protease inhibitor for example in liquid form. Besides the mixing step and the plodding step, the process may further comprise the steps of milling, extruding, cutting, stamping, cooling and/or wrapping.

### Granules

The present invention also relates to enzyme granules/particles comprising alpha-mannan degrading enzymes. In an embodiment, the granule comprises a core, and optionally one or more coatings (outer layers) surrounding the core.

The core may have a diameter, measured as equivalent spherical diameter (volume based average particle size), of 20-2000 μm, particularly 50-1500 μm, 100-1500 μm or 250-1200 μm.

In an embodiment, the core comprises one or more polypeptides having alpha-mannan degrading activity.

The core may include additional materials such as fillers, fiber materials (cellulose or synthetic fibers), stabilizing agents, solubilizing agents, suspension agents, viscosity regulating agents, light spheres, plasticizers, salts, lubricants and fragrances.

The core may include a binder, such as synthetic polymer, wax, fat, or carbohydrate.

The core may include a salt of a multivalent cation, a reducing agent, an antioxidant, a peroxide decomposing catalyst and/or an acidic buffer component, typically as a homogenous blend.

The core may include an inert particle with the enzyme absorbed into it, or applied onto the surface, e.g., by fluid bed coating.

The core may have a diameter of 20-2000 μm, particularly 50-1500 μm, 100-1500 μm or 250-1200 μm.

The core may be surrounded by at least one coating, e.g., to improve the storage stability, to reduce dust formation during handling, or for coloring the granule. The optional coating(s) may include a salt coating, or other suitable coating materials, such as polyethylene glycol (PEG), methyl hydroxy-propyl cellulose (MHPC) and polyvinyl alcohol (PVA).

The coating may be applied in an amount of at least 0.1% by weight of the core, e.g., at least 0.5%, at least 1%, at least 5%, at least 10%, or at least 15%. The amount may be at most 100%, 70%, 50%, 40% or 30%.

The coating is preferably at least 0.1 μm thick, particularly at least 0.5 μm, at least 1 μm or at least 5 μm. In some embodiments, the thickness of the coating is below 100 μm, such as below 60 μm, or below 40 μm.

The coating should encapsulate the core unit by forming a substantially continuous layer. A substantially continuous layer is to be understood as a coating having few or no holes, so that the core unit it is encapsulating/enclosing has few or none uncoated areas. The layer or coating should, in particular, be homogeneous in thickness.

The coating can further contain other materials as known in the art, e.g., fillers, antisticking agents, pigments, dyes, plasticizers and/or binders, such as titanium dioxide, kaolin, calcium carbonate or talc.

A salt coating may comprise at least 60% by weight of a salt, e.g., at least 65%, at least 70%, at least 75%, at least 80%, at least 85%, at least 90%, at least 95% or at least 99% by weight.

To provide acceptable protection, the salt coating is preferably at least 0.1 μm thick, e.g., at least 0.5 μm, at least 1 μm, at least 2 μm, at least 4 μm, at least 5 μm, or at least 8 μm. In a particular embodiment, the thickness of the salt coating is below 100 μm, such as below 60 μm, or below 40 μm.

The salt may be added from a salt solution where the salt is completely dissolved or from a salt suspension wherein the fine particles are less than 50 μm, such as less than 10 μm or less than 5 μm.

The salt coating may comprise a single salt or a mixture of two or more salts. The salt may be water soluble, in particular, having a solubility at least 0.1 g in 100 g of water at 20° C., preferably at least 0.5 g per 100 g water, e.g., at least 1 g per 100 g water, e.g., at least 5 g per 100 g water.

The salt may be an inorganic salt, e.g., salts of sulfate, sulfite, phosphate, phosphonate, nitrate, chloride or carbonate or salts of simple organic acids (less than 10 carbon atoms, e.g., 6 or less carbon atoms) such as citrate, malonate or acetate. Examples of cations in these salts are alkali or earth alkali metal ions, the ammonium ion or metal ions of the first transition series, such as sodium, potassium, magnesium, calcium, zinc or aluminum. Examples of anions include chloride, bromide, iodide, sulfate, sulfite, bisulfite, thiosulfate, phosphate, monobasic phosphate, dibasic phosphate, hypophosphite, dihydrogen pyrophosphate, tetraborate, borate, carbonate, bicarbonate, metasilicate, citrate, malate, maleate, malonate, succinate, lactate, formate, acetate, butyrate, propionate, benzoate, tartrate, ascorbate or gluconate. In particular alkali- or earth alkali metal salts of sulfate, sulfite, phosphate, phosphonate, nitrate, chloride or carbonate or salts of simple organic acids such as citrate, malonate or acetate may be used.

The salt in the coating may have a constant humidity at 20° C. above 60%, particularly above 70%, above 80% or above 85%, or it may be another hydrate form of such a salt (e.g., anhydrate).

The salt coating may be as described in WO 00/01793 or WO 2006/034710.

Specific examples of suitable salts are NaCl (CH_{20° C.}=76%), Na₂CO₃ (CH_{20° C.}=92%), NaNO₃ (CH_{20° C.}=73%), Na₂HPO₄ (CH_{20° C.}=95%), Na₃PO₄ (CH_{25° C.}=92%), NH₄Cl (CH_{20° C.}=79.5%), (NH₄)₂HPO₄ (CH_{20° C.}=93.0%), NH₄H₂PO₄ (CH_{20° C.}=93.1%), (NH₄)₂SO₄ (CH_{20° C.}=81.1%), KCl (CH_{20° C.}=85%), K₂HPO₄ (CH_{20° C.}=92%), KH₂PO₄ (CH_{20° C.}=96.5%), KNO₃ (CH_{20° C.}=93.5%), Na₂SO₄ (CH_{20° C.}=93%), K₂SO₄ (CH_{20° C.}=98%), KHSO₄ (CH_{20° C.}=86%), MgSO₄ (CH_{20° C.}=90%), ZnSO₄ (CH_{20° C.}=90%) and sodium citrate (CH_{25° C.}=86%). Other examples include NaH₂PO₄, (NH₄)H₂PO₄, CuSO₄, Mg(NO₃)₂ and magnesium acetate.

The salt may be in anhydrous form, or it may be a hydrated salt, i.e. a crystalline salt hydrate with bound water(s) of crystallization, such as described in WO 99/32595. Specific examples include anhydrous sodium sulfate (Na₂SO₄), anhydrous magnesium sulfate (MgSO₄), magnesium sulfate heptahydrate (MgSO₄·7H₂O), zinc sulfate heptahydrate (ZnSO₄·7H₂O), sodium phosphate dibasic heptahydrate (Na₂HPO₄·7H₂O), magnesium nitrate hexahydrate (Mg(NO₃)₂(6H₂O)), sodium citrate dihydrate and magnesium acetate tetrahydrate.

Preferably the salt is applied as a solution of the salt, e.g., using a fluid bed.

The coating materials can be waxy coating materials and film-forming coating materials. Examples of waxy coating materials are poly(ethylene oxide) products (polyethyleneglycol, PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591.

The granule may optionally have one or more additional coatings. Examples of suitable coating materials are polyethylene glycol (PEG), methyl hydroxy-propyl cellulose (MHPC) and polyvinyl alcohol (PVA). Examples of enzyme granules with multiple coatings are described in WO 93/07263 and WO 97/23606.

The core can be prepared by granulating a blend of the ingredients, e.g., by a method comprising granulation techniques such as crystallization, precipitation, pan-coating, fluid bed coating, fluid bed agglomeration, rotary atomization, extrusion, prilling, spheronization, size reduction methods, drum granulation, and/or high shear granulation.

Methods for preparing the core can be found in the Handbook of Powder Technology; Particle size enlargement by C. E. Capes; Volume 1; 1980; Elsevier. Preparation methods include known feed and granule formulation technologies, e.g.,
(a) Spray dried products, wherein a liquid enzyme-containing solution is atomized in a spray drying tower to form small droplets which during their way down the drying tower dry to form an enzyme-containing particulate material. Very small particles can be produced this way (Michael S. Showell (editor); Powdered detergents; Surfactant Science Series; 1998; vol. 71; page 140-142; Marcel Dekker).
(b) Layered products, wherein the enzyme is coated as a layer around a pre-formed inert core particle, wherein an enzyme-containing solution is atomized, typically in a fluid bed apparatus wherein the pre-formed core particles are fluidized, and the enzyme-containing solution adheres to the core particles and dries up to leave a layer of dry enzyme on the surface of the core particle. Particles of a desired size can be obtained this way if a useful core particle of the desired size can be found. This type of product is described in, e.g., WO 97/23606.
(c) Absorbed core particles, wherein rather than coating the enzyme as a layer around the core, the enzyme is absorbed onto and/or into the surface of the core. Such a process is described in WO 97/39116.
(d) Extrusion or pelletized products, wherein an enzyme-containing paste is pressed to pellets or under pressure is extruded through a small opening and cut into particles which are subsequently dried. Such particles usually have a considerable size because of the material in which the extrusion opening is made (usually a plate with bore holes) sets a limit on the allowable pressure drop over the extrusion opening. Also, very high extrusion pressures when using a small opening increase heat generation in the enzyme paste, which is harmful to the enzyme (Michael S. Showell (editor); Powdered detergents; Surfactant Science Series; 1998; vol. 71; pages 140-142; Marcel Dekker).
(e) Prilled products, wherein an enzyme-containing powder is suspended in molten wax and the suspension is sprayed, e.g., through a rotating disk atomizer, into a cooling chamber where the droplets quickly solidify (Michael S. Showell (editor); Powdered detergents; Surfactant Science Series; 1998; vol. 71; page 140-142; Marcel Dekker). The product obtained is one wherein the enzyme is uniformly distributed throughout an inert material instead of being concentrated on its surface. U.S. Pat. Nos. 4,016,040 and 4,713,245 describe this technique.
(f) Mixer granulation products, wherein an enzyme-containing liquid is added to a dry powder composition of conventional granulating components. The liquid and the powder in a suitable proportion are mixed and as the moisture of the liquid is absorbed in the dry powder, the components of the dry powder will start to adhere and agglomerate and particles will build up, forming granulates comprising the enzyme. Such a process is described in U.S. Pat. No. 4,106,991 and related documents EP 170360, EP 304332, EP 304331, WO 90/09440 and WO 90/09428. In a particular product of this process, various high-shear mixers can be used as granulators. Granulates consisting of enzyme, fillers and binders etc. are mixed with cellulose fibers to reinforce the particles to produce a so-called T-granulate. Reinforced particles, are more robust, and release less enzymatic dust.
(g) Size reduction, wherein the cores are produced by milling or crushing of larger particles, pellets, tablets, briquettes etc. containing the enzyme. The wanted core particle fraction is obtained by sieving the milled or crushed product. Over and undersized particles can be recycled. Size reduction is described in Martin Rhodes (editor); Principles of Powder Technology; 1990; Chapter 10; John Wiley & Sons.
(h) Fluid bed granulation. Fluid bed granulation involves suspending particulates in an air stream and spraying a liquid onto the fluidized particles via nozzles. Particles hit by spray droplets get wetted and become tacky. The tacky particles collide with other particles and adhere to them to form a granule.
(i) The cores may be subjected to drying, such as in a fluid bed drier. Other known methods for drying granules in the feed or enzyme industry can be used by the skilled person. The drying preferably takes place at a product temperature of from 25 to 90° C. For some enzymes, it is important the cores comprising the enzyme contain a low amount of water before coating with the salt. If water sensitive enzymes are coated with a salt before excessive water is removed, it will be trapped within the core and may affect the activity of the enzyme negatively. After drying, the cores preferably contain 0.1-10% w/w water.

Non-dusting granulates may be produced, e.g., as disclosed in U.S. Pat. Nos. 4,106,991 and 4,661,452 and may optionally be coated by methods known in the art.

The granulate may further one or more additional enzymes. Each enzyme will then be present in more granules securing a more uniform distribution of the enzymes, and also reduces the physical segregation of different enzymes due to different particle sizes. Methods for producing multi-enzyme co-granulates is disclosed in the ip.com disclosure IPCOM000200739D.

Another example of formulation of enzymes by the use of co-granulates is disclosed in WO 2013/188331.

The present invention also relates to protected enzymes prepared according to the method disclosed in EP 238,216.

In an embodiment, the granule further comprises one or more additional enzymes, e.g., hydrolase, isomerase, ligase, lyase, oxidoreductase, and transferase. The one or more additional enzymes are preferably selected from the group consisting of acetylxylan esterase, acylglycerol lipase, amylase, alpha-amylase, beta-amylase, arabinofuranosidase, cellobiohydrolases, cellulase, feruloyl esterase, galactanase, alpha-galactosidase, beta-galactosidase, beta-glucanase, beta-glucosidase, lysophospholipase, lysozyme, beta-mannosidase (mannanase), phytase, phospholipase A1, phospholipase A2, phospholipase D, protease, pullulanase, pectin esterase, triacylglycerol lipase, xylanase, beta-xylosidase or any combination thereof.

### Liquid Formulations

The present invention also relates to liquid compositions comprising alpha-mannan degrading enzymes. The composition may comprise an enzyme stabilizer (examples of which include polyols such as propylene glycol or glycerol, sugar or sugar alcohol, lactic acid, reversible protease inhibitor, boric acid, or a boric acid derivative, e.g., an aromatic borate ester, or a phenyl boronic acid derivative such as 4-formylphenyl boronic acid).

In some embodiments, filler(s) or carrier material(s) are included to increase the volume of such compositions. Suitable filler or carrier materials include, but are not limited to, various salts of sulfate, carbonate and silicate as well as talc, clay and the like. Suitable filler or carrier materials for liquid compositions include, but are not limited to water or low molecular weight primary and secondary alcohols including polyols and diols. Examples of such alcohols include, but are not limited to, methanol, ethanol, propanol and isopropanol. In some embodiments, the compositions contain from about 5% to about 90% of such materials.

In an aspect, the present invention relates to liquid formulations comprising:
(A) 0.001% to 25% w/w of one or more polypeptides having alpha-mannan degrading activity of the present invention; and
(B) water.

In another embodiment, the liquid formulation comprises 20% to 80% w/w of polyol. In one embodiment, the liquid formulation comprises 0.001% to 2.0% w/w preservative.

In another embodiment, the invention relates to liquid formulations comprising:
(A) 0.001% to 25% w/w of one or more polypeptides having alpha-mannan degrading activity of the present invention;
(B) 20% to 80% w/w of polyol;
(C) optionally 0.001% to 2.0% w/w preservative; and
(D) water.

In another embodiment, the invention relates to liquid formulations comprising:
(A) 0.001% to 25% w/w of one or more polypeptides having alpha-mannan degrading activity of the present invention;
(B) 0.001% to 2.0% w/w preservative;
(C) optionally 20% to 80% w/w of polyol; and
(D) water.

In another embodiment, the liquid formulation comprises one or more formulating agents, such as a formulating agent selected from the group consisting of polyol, sodium chloride, sodium benzoate, potassium sorbate, sodium sulfate, potassium sulfate, magnesium sulfate, sodium thiosulfate, calcium carbonate, sodium citrate, dextrin, glucose, sucrose, sorbitol, lactose, starch, PVA, acetate and phosphate, preferably selected from the group consisting of sodium sulfate, dextrin, cellulose, sodium thiosulfate, kaolin and calcium carbonate. In one embodiment, the polyols is selected from the group consisting of glycerol, sorbitol, propylene glycol (MPG), ethylene glycol, diethylene glycol, triethylene glycol, 1,2-propylene glycol or 1,3-propylene glycol, dipropylene glycol, polyethylene glycol (PEG) having an average molecular weight below about 600 and polypropylene glycol (PPG) having an average molecular weight below about 600, more preferably selected from the group consisting of glycerol, sorbitol and propylene glycol (MPG) or any combination thereof.

In another embodiment, the liquid formulation comprises 20%-80% polyol (i.e., total amount of polyol), e.g., 25%-75% polyol, 30%-70% polyol, 35%-65% polyol, or 40%-60% polyol. In one embodiment, the liquid formulation comprises 20%-80% polyol, e.g., 25%-75% polyol, 30%-70% polyol, 35%-65% polyol, or 40%-60% polyol, wherein the polyol is selected from the group consisting of glycerol, sorbitol, propylene glycol (MPG), ethylene glycol, diethylene glycol, triethylene glycol, 1,2-propylene glycol or 1,3-propylene glycol, dipropylene glycol, polyethylene glycol (PEG) having an average molecular weight below about 600 and polypropylene glycol (PPG) having an average molecular weight below about 600. In one embodiment, the liquid formulation comprises 20%-80% polyol (i.e., total amount of polyol), e.g., 25%-75% polyol, 30%-70% polyol, 35%-65% polyol, or 40%-60% polyol, wherein the polyol is selected from the group consisting of glycerol, sorbitol and propylene glycol (MPG).

In another embodiment, the preservative is selected from the group consisting of sodium sorbate, potassium sorbate, sodium benzoate and potassium benzoate or any combination thereof. In one embodiment, the liquid formulation comprises 0.02% to 1.5% w/w preservative, e.g., 0.05% to 1.0% w/w preservative or 0.1% to 0.5% w/w preservative. In one embodiment, the liquid formulation comprises 0.001% to 2.0% w/w preservative (i.e., total amount of preservative), e.g., 0.02% to 1.5% w/w preservative, 0.05% to 1.0% w/w preservative, or 0.1% to 0.5% w/w preservative, wherein the preservative is selected from the group consisting of sodium sorbate, potassium sorbate, sodium benzoate and potassium benzoate or any combination thereof.

In another embodiment, the liquid formulation further comprises one or more additional enzymes, e.g., hydrolase, isomerase, ligase, lyase, oxidoreductase, and transferase. The one or more additional enzymes are preferably selected from the group consisting of acetylxylan esterase, acylglycerol lipase, amylase, alpha-amylase, beta-amylase, arabinofuranosidase, cellobiohydrolases, cellulase, feruloyl esterase, galactanase, alpha-galactosidase, beta-galactosidase, beta-glucanase, beta-glucosidase, lysophospholipase, lysozyme, beta-mannosidase (mannanase), phytase, phospholipase A1, phospholipase A2, phospholipase D, protease, pullulanase, pectin esterase, triacylglycerol lipase, xylanase, beta-xylosidase or any combination thereof.

### Uses

### Washing Method

The detergent compositions of the present invention are ideally suited for use in laundry applications. Accordingly, the present invention includes a method for laundering a fabric. The method comprises the steps of contacting a fabric to be laundered with a cleaning laundry solution comprising the detergent composition according to the invention. The fabric may comprise any fabric capable of being laundered in normal consumer use conditions. The solution preferably has a pH of from about 5.5 to about 8. The compositions may be employed at concentrations of from about 100 ppm, preferably 500 ppm to about 15,000 ppm in solution. The water temperatures typically range from about 5° C. to about 90° C., including about 10° C., about 15° C., about 20° C., about 25° C., about 30° C., about 35° C., about 40° C., about 45° C., about 50° C., about 55° C., about 60° C., about 65° C., about 70° C., about 75° C., about 80° C., about 85° C. and about 90° C. The water to fabric ratio is typically from about 1:1 to about 30:1.

In particular embodiments, the washing method is conducted at a pH of from about 5.0 to about 11.5, or in alternative embodiments, even from about 6 to about 10.5, such as about 5 to about 11, about 5 to about 10, about 5 to about 9, about 5 to about 8, about 5 to about 7, about 5.5 to about 11, about 5.5 to about 10, about 5.5 to about 9, about 5.5 to about 8, about 5.5. to about 7, about 6 to about 11, about 6 to about 10, about 6 to about 9, about 6 to about 8, about 6 to about 7, about 6.5 to about 11, about 6.5 to about 10, about 6.5 to about 9, about 6.5 to about 8, about 6.5 to about 7, about 7 to about 11, about 7 to about 10, about 7 to about 9, or about 7 to about 8, preferably about 5.5 to about 9, and more preferably about 6 to about 8.

In particular embodiments, the washing method is conducted at a degree of hardness of from about 0° dH to about 30° dH, such as about 1° dH, about 2° dH, about 3° dH, about 4° dH, about 5° dH, about 6° dH, about 7° dH, about 8° dH, about 9° dH, about 10° dH, about 11° dH, about 12° dH, about 13° dH, about 14° dH, about 15° dH, about 16° dH, about 17° dH, about 18° dH, about 19° dH, about 20° dH, about 21° dH, about 22° dH, about 23° dH, about 24° dH, about 25° dH, about 26° dH, about 27° dH, about 28° dH, about 29° dH, about 30° dH. Under typical European wash conditions, the degree of hardness is about 15° dH, under typical US wash conditions about 6° dH, and under typical Asian wash conditions, about 3° dH.

The present invention relates to a method of cleaning a fabric, a dishware or hard surface with a detergent composition comprising an alpha-mannan degrading enzyme.

A preferred embodiment concerns a method of cleaning, said method comprising the steps of: contacting an object with a cleaning composition comprising an alpha-mannan degrading enzyme under conditions suitable for cleaning said object. In a preferred embodiment the cleaning composition is a detergent composition and the process is a laundry or a dish wash process.

Still another embodiment relates to a method for removing stains from fabric which comprises contacting said a fabric with a composition comprising an alpha-mannan degrading enzyme under conditions suitable for cleaning said object.

### Low Temperature Uses

One embodiment of the invention concerns a method of doing laundry, dish wash or industrial cleaning comprising contacting a surface to be cleaned with an alpha-mannan degrading enzyme, and wherein said laundry, dish wash, industrial or institutional cleaning is performed at a temperature of about 40° C. or below. One embodiment of the invention relates to the use of an alpha-mannan degrading enzyme in laundry, dish wash or a cleaning process wherein the temperature in laundry, dish wash, industrial cleaning is about 40° C. or below

In another embodiment, the invention concerns the use of an alpha-mannan degrading enzyme in a protein removing process, wherein the temperature in the protein removing process is about 40° C. or below.

In each of the above-identified methods and uses, the wash temperature is about 40° C. or below, such as about 39° C. or below, such as about 38° C. or below, such as about 37° C. or below, such as about 36° C. or below, such as about 35° C. or below, such as about 34° C. or below, such as about 33° C. or below, such as about 32° C. or below, such as about 31° C. or below, such as about 30° C. or below, such as about 29° C. or below, such as about 28° C. or below, such as about 27° C. or below, such as about 26° C. or below, such as about 25° C. or below, such as about 24° C. or below, such as about 23° C. or below, such as about 22° C. or below, such as about 21° C. or below, such as about 20° C. or below, such as about 19° C. or below, such as about 18° C. or below, such as about 17° C. or below, such as about 16° C. or below, such as about 15° C. or below, such as about 14° C. or below, such as about 13° C. or below, such as about 12° C. or below, such as about 11° C. or below, such as about 10° C. or below, such as about 9° C. or below, such as about 8° C. or below, such as about 7° C. or below, such as about 6° C. or below, such as about 5° C. or below, such as about 4° C. or below, such as about 3° C. or below, such as about 2° C. or below, such as about 1° C. or below.

In another preferred embodiment, the wash temperature is in the range of about 5-40° C., such as about 5-30° C., about 5-20° C., about 5-10° C., about 10-40° C., about 10-30° C., about 10-20° C., about 15-40° C., about 15-30° C., about 15-20° C., about 20-40° C., about 20-30° C., about 25-40° C., about 25-30° C., or about 30-40° C. In particular preferred embodiments the wash temperature is about 20° C., about 30° C., or about 40° C.

### Preventing, Reducing or Removing a Biofilm

Biofilm can develop on textile when microorganisms are present on an item and stick together on the item. Some microorganisms tend to adhere to the surface of items such as textiles. Some microorganisms adhere to such surfaces and form a biofilm on the surface. The biofilm may be sticky and the adhered microorganisms and/or the biofilm may be difficult to remove. Furthermore, the biofilm adhere soil due to the sticky nature of the biofilm. The commercial laundry detergent compositions available on the marked do not remove such adhered microorganisms or biofilm.

In an embodiment, the present invention concerns the use of an alpha-mannan degrading enzyme for preventing, reducing or removing a biofilm from an item, wherein the polypeptide is obtained from a fungal source and wherein the item is a textile. In one embodiment, alpha-mannan degrading enzyme is used for preventing, reducing or removing the stickiness of an item.

### Food Processing and Animal Feed

Accordingly, the present invention relates to an animal feed composition and/or animal feed additive composition and/or pet food comprising an alpha-mannan degrading enzyme.

The present invention further relates to a method for preparing such animal feed composition and/or animal feed additive composition and/or pet food comprising mixing an alpha-mannan degrading enzyme with one or more animal feed ingredients and/or animal feed additive ingredients and/or pet food ingredients.

Furthermore, the present invention relates to the use of an alpha-mannan degrading enzyme in the preparation of an animal feed composition and/or animal feed additive composition and/or pet food.

Degrading a Cellulosic Material and/or Producing a Fermentation Product

An alpha-mannan degrading enzyme may be used for degrading a cellulosic material, for producing a fermentation product and for fermenting a cellulosic material e.g., in a process for producing a fermentation product, comprising: (a) saccharifying a cellulosic material with an enzyme composition, wherein the enzyme composition comprises an alpha-mannan degrading enzyme; (b) fermenting the saccharified cellulosic material with one or more fermenting microorganisms to produce the fermentation product; and (c) recovering the fermentation product from the fermentation. The cellulosic material may be pretreated before saccharification. In one embodiment, the enzyme composition comprises one or more enzymes selected from the group consisting of cellulase, AA9 polypeptide, hemicellulase, esterase, expansin, ligninolytic enzyme, oxidoreductase, pectinase, protease, and swollenin.

In another embodiment, the invention relates to a process of fermenting a cellulosic material, comprising: fermenting the cellulosic material with one or more fermenting microorganisms, wherein the cellulosic material is saccharified with an enzyme composition comprising an alpha-mannan degrading enzyme. The cellulosic material may be pretreated before saccharification. In one embodiment, the enzyme composition comprises one or more enzymes selected from the group consisting of cellulase, AA9 polypeptide, hemicellulase, esterase, expansin, ligninolytic enzyme, oxidoreductase, pectinase, protease, and swollenin.

### Use for Fermented Beverages

In one aspect, the invention relates to a method of preparing a fermented beverage, such as beer or wine, comprising mixing an alpha-mannan degrading enzyme with malt and/or adjunct.

Another aspect concerns a method of providing a fermented beverage comprising the step of contacting a mash and/or a wort with an alpha-mannan degrading enzyme.

In the context of the present invention, the term “fermented beverage” is meant to comprise any beverage such as wine or beer produced by a method comprising a fermentation process, such as a microbial, bacterial and/or yeast fermentation.

In an aspect of the invention the fermented beverage is beer. The term “beer” is meant to comprise any fermented wort produced by fermentation/brewing of a starch-containing plant material.

Often, beer is produced from malt or adjunct, or any combination of malt and adjunct as the starch-containing plant material. As used herein the term “malt” is understood as any malted cereal grain, such as malted barley or wheat.

As used herein the term “adjunct” refers to any starch and/or sugar containing plant material which is not malt, such as barley or wheat malt. As examples of adjuncts, mention can be made of materials such as common corn grits, refined corn grits, brewer's milled yeast, rice, sorghum, refined corn starch, barley, barley starch, dehusked barley, wheat, wheat starch, torrified cereal, cereal flakes, rye, oats, potato, tapioca, cassava and syrups, such as corn syrup, sugar cane syrup, inverted sugar syrup, barley and/or wheat syrups, and the like may be used as a source of starch

As used herein, the term “mash” refers to an aqueous slurry of any starch and/or sugar containing plant material such as grist, e. g. comprising crushed barley malt, crushed barley, and/or other adjunct or a combination hereof, mixed with water later to be separated into wort and spent grains.

As used herein, the term “wort” refers to the unfermented liquor run-off following extracting the grist during mashing.

### Treating Coffee Extracts

An alpha-mannan degrading enzyme may also be used for hydrolyzing galactomannans present in liquid coffee extracts. In certain preferred embodiments, an alpha-mannan degrading enzyme is used to inhibit gel formation during freeze drying of liquid coffee extracts. The decreased viscosity of the extract reduces the energy consumption during drying. In certain other preferred embodiments, an alpha-mannan degrading enzyme is applied in an immobilized form in order to reduce enzyme consumption and avoid contamination of the coffee extract. This use is further disclosed in EP 676 145.

In general terms the coffee extract is incubated in the presence of an alpha-mannan degrading enzyme thereof under conditions suitable for hydrolyzing galactomannans present in liquid coffee extract.

Thus in one embodiment, then invention relates to a process for producing a coffee extract, comprising the steps:
(a) providing roast and ground coffee beans;
(b) adding to said coffee beans water and an alpha-mannan degrading enzyme;
(c) incubating to make an aqueous coffee extract; and
(d) separating the coffee extract from the extracted coffee beans.

### Use in Bakery Food Products

In another aspect, the invention relates to a method of preparing baked products comprising adding an alpha-mannan degrading enzyme to a dough, followed by baking the dough.

Examples of baked products are well known to those skilled in the art and include breads, rolls, puff pastries, sweet fermented doughs, buns, cakes, crackers, cookies, biscuits, waffles, wafers, tortillas, breakfast cereals, extruded products, and the like.

An alpha-mannan degrading enzyme may be added to dough as part of a bread improver composition. Bread improvers are compositions containing a variety of ingredients, which improve dough properties and the quality of bakery products, e.g. bread and cakes. Bread improvers are often added in industrial bakery processes because of their beneficial effects e.g. the dough stability and the bread texture and volume. Bread improvers usually contain fats and oils as well as additives like emulsifiers, enzymes, antioxidants, oxidants, stabilizers and reducing agents. In addition to the mannanase of the invention, other enzymes which may also be present in the bread improver including amylases, hemicellulases, amylolytic complexes, lipases, proteases, xylanases, pectinases, pullulanases, non-starch polysaccharide degrading enzymes and redox enzymes like glucose oxidase, lipoxygenase or ascorbic acid oxidase.

In one aspect, an alpha-mannan degrading enzyme may be added to dough as part of a bread improver composition which also comprises a glucomannan and/or galactomannan source such as konjac gum, guar gum, locust bean gum (Ceratonia siliqua), copra meal, ivory nut mannan (Phyteleohas macrocarpa), seaweed mannan extract, coconut meal, and the cell wall of brewers yeast (may be dried, or used in the form of brewers yeast extract).

A further aspect of the invention relates to the use of an alpha-mannan degrading enzyme in dough to improve dough tolerance, flexibility and stickiness. Preferably the dough to which the mannanase of the invention may be added is not a pure wheat flour dough, but comprises bran or oat, rice, millet, maize, or legume flour in addition to or instead of pure wheat flour.

A yet further aspect of the invention relates to the use of an alpha-mannan degrading enzyme in dough to improve the crumb structure and retard staling in the final baked product, such as bread.

### Use in Dairy Food Products

In one aspect of the current invention, an alpha-mannan degrading enzyme may be added to milk or any other dairy product, for example, to which has also been added a glucomannan and/or galactomannan.

In one aspect, the invention relates to a method of preparing milk or dairy products comprising adding to the milk or dairy product (a) glucomannan, galactomannan and/or galactoglucomannan and (b) an alpha-mannan degrading enzyme.

In one aspect an alpha-mannan degrading enzyme in combination with any glucomannan or galactomannan prior to or following addition to a dairy based foodstuff to produce a dairy based foodstuff comprising prebiotic mannan hydrolysates. In a further aspect of the invention the thus produced mannooligosacharide-containing dairy product is capable of increasing the population of beneficial human intestinal microflora, and in a yet further aspect of the current invention the dairy based foodstuff may comprise an alpha-mannan degrading enzyme together with any source of glucomannan and/or galactomannan and/or galactoglucomannan, and a dose sufficient for inoculation of at least one strain of bacteria (such as Bifidobacteria or Lactobacillus) known to be of benefit in the human large intestine. Preferably said dairy-based foodstuff is a yoghurt or milk drink.

### Paper Pulp Bleaching

The alpha-mannan degrading enzyme may further be used in the enzyme aided bleaching of paper pulps such as chemical pulps, semi-chemical pulps, kraft pulps, mechanical pulps or pulps prepared by the sulfite method. Thus, the invention relates to a method of bleaching paper pulps comprising incubating the paper pulp with an alpha-mannan degrading enzyme.

In some embodiments, the pulps are chlorine free pulps bleached with oxygen, ozone, peroxide or peroxyacids. In some embodiments, an alpha-mannan degrading enzyme is used in enzyme aided bleaching of pulps produced by modified or continuous pulping methods that exhibit low lignin contents. In some other embodiments, an alpha-mannan degrading enzyme is applied alone or preferably in combination with xylanase and/or endoglucanase and/or alpha-galactosidase and/or cellobiohydrolase enzymes.

The invention is further defined in the following paragraphs:

Paragraph 1. A cleaning composition comprising:
(a) at least one polypeptide having alpha-mannan degrading activity or a variant or a fragment thereof having alpha-mannan degrading activity; and
(b) at least one cleaning component, preferably selected from a surfactant, a builder, a bleach component, a polymer, a dispersing agent and/or an additional enzyme.

Paragraph 2. The cleaning composition of paragraph 1, wherein the polypeptide has alpha-mannanase and/or alpha-mannosidase activity.

Paragraph 3. The cleaning composition of any preceding paragraph, wherein the polypeptide having alpha-mannan degrading activity belongs to glycosyl hydrolase family 76 (GH76), glycosyl hydrolase family 92 (GH92), glycosyl hydrolase family 99 (GH99), in particular to glycosyl hydrolase family 76.

Paragraph 4. The cleaning composition of any preceding paragraph, wherein the polypeptide comprises one or more of the motifs [YND]DD[QINLEM] (SEQ ID NO: 60) and GG[ILMV]X[WS](SEQ ID NO: 61).

Paragraph 5. The cleaning composition of any preceding paragraph, wherein the polypeptide further comprises the motif [RK][NLT]XXX[NTV]XP[GTLYISAVFNM] (SEQ ID NO: 64).

Paragraph 6. The cleaning composition of any of paragraphs 1-5, wherein the polypeptide is of the KNTPA clade and is of bacterial origin.

Paragraph 7. The cleaning composition of any of paragraphs 1-4, wherein the polypeptide further comprises the motif GA]XX[AVL][ML]X[MA][ATV][EATV] (SEQ ID NO: 62) or the motif LA[EQ]X[VL][YF] (SEQ ID NO: 63).

Paragraph 8. The cleaning composition of any of paragraphs 1-4 or 7, wherein the polypeptide is of the AMXAAE clade and is of fungal origin.

Paragraph 9. The cleaning composition of any of paragraphs 1-3, wherein the polypeptide comprises the motif N[EQD][WFY][HG]E (SEQ ID NO: 66).

Paragraph 10. The cleaning composition of any preceding paragraph, comprising at least two polypeptides having alpha-mannan degrading activity.

Paragraph 11. The cleaning composition of any preceding paragraph, comprising a polypeptide belonging to GH family 76 and a polypeptide belonging to GH family 92; a polypeptide belonging to GH family 76 and a polypeptide belonging to GH family 99; a polypeptide belonging to GH family 92 and a polypeptide belonging to GH family 99; in particular, a polypeptide belong to GH family 76 and a polypeptide belonging to GH family 99.

Paragraph 12. The cleaning composition of any preceding paragraph, wherein the polypeptide having alpha-mannan degrading activity is microbial, preferably obtained from bacteria or fungi.

Paragraph 13. The cleaning composition of any preceding paragraph, wherein the polypeptide having alpha-mannan degrading activity is obtained from a strain of Bacillus, e.g., Bacillus acidicola, Bacillus novalis, or Bacillus sp., a strain of Paenibacillus, e.g., Paenibacillus glycanilyticus, or Paenibacillus sp., or from a strain of Aspergillus, e.g., Aspergillus aculeatus, or from a strain of Humicola, e.g., Humicola insolens.

Paragraph 14. The cleaning composition of any preceding paragraph, wherein the polypeptide is selected from the group consisting of:
(a) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 3;
(b) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 6;
(c) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 9;
(d) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 12;
(e) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 15;
(f) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 18;
(g) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 21;
(h) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 24;
(i) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 27;
(j) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 30;
(k) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 33;
(l) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 36;
(m) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 39;
(n) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 42;
(o) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 45;
(p) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 48;
(q) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 51;
(r) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 54;
(s) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 57.

Paragraph 15. The cleaning composition of any preceding paragraph, wherein the polypeptide is selected from the group consisting of:
(a) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 3;
(b) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 6;
(c) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 9;
(d) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 12;
(e) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 15;
(f) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 18;
(g) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 21;
(h) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 24; and
(i) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 57.

Paragraph 16. The cleaning composition of any preceding paragraph, wherein the polypeptide is selected from the group consisting of:
(a) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 33;
(b) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 36;
(c) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 39;
(d) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 42;
(e) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 45;
(f) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 48;
(g) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 51;
(h) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 54.

Paragraph 17. The cleaning composition of any preceding paragraph, comprising at least 0.001 ppm of the polypeptide having alpha-mannan degrading activity.

Paragraph 18. The cleaning composition of any preceding paragraph, comprising from 0.2 wt % to 20 wt % surfactant.

Paragraph 19. The cleaning composition of any preceding paragraph, further comprising a polypeptide having beta-mannanase activity.

Paragraph 20. The cleaning composition of paragraph 19, wherein the polypeptide having beta-mannanase activity is selected from the group consisting of:
(a) a polypeptide having at least 59%, at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 67;
(b) a polypeptide having at least 59%, at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 68;
(c) a polypeptide having at least 59%, at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 69.

Paragraph 21. The cleaning composition of paragraph 20, wherein the polypeptide having alpha-mannan degrading activity is a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 24.

Paragraph 22. The cleaning composition of paragraph 20, wherein the polypeptide having alpha-mannan degrading activity is a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 57.

Paragraph 23. The cleaning composition of paragraph 20, wherein the polypeptide having alpha-mannan degrading activity is a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 30.

Paragraph 24. The cleaning composition of paragraph 20, wherein the polypeptide having alpha-mannan degrading activity is a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 3.

Paragraph 25. A granule comprising a core particle and one or more coatings and a polypeptide having alpha-mannan degrading activity.

Paragraph 26. A liquid composition comprising a polyol and a polypeptide having alpha-mannan degrading activity.

Paragraph 27. Use of the cleaning composition of any of paragraphs 1-24, the granule of paragraph 25 or the liquid composition of paragraph 26 for laundering, washing or cleaning a textile and/or a hard surface (such as dish wash).

Paragraph 28. Use of the cleaning composition of any of paragraphs 1-24, the granule of paragraph 25 or the liquid composition of paragraph 26 for
(a) preventing, reducing or removing stickiness of the item;
(b) preventing, reducing or removing biofilm or biofilm components;
(c) preventing, reducing or removing redeposition of soil during a wash cycle;
(d) preventing, reducing or removing adherence of soil to the item;
(e) maintaining or improving whiteness of the item; or
(f) preventing, reducing or removing malodor from the item,
wherein the item is a textile.

Paragraph 29. A process for degrading alpha-mannan, comprising applying the cleaning composition of any of paragraph 1-24, the granule of paragraph 25 or the liquid composition of paragraph 26 to the alpha-mannan.

Paragraph 30. A method of cleaning an item, comprising the steps of:
(a) contacting the item with a cleaning composition according to any of paragraphs 1-24, the granule of paragraph 25 or the liquid composition of paragraph 26; and
(b) and optionally rinsing the item, wherein the item is preferably a textile or a hard surface.

Paragraph 31. An isolated or purified polypeptide having alpha-mannan degrading activity, wherein the polypeptide having alpha-mannan degrading activity belongs to glycosyl hydrolase family 76 (GH76), glycosyl hydrolase family 92 (GH92), glycosyl hydrolase family 99 (GH99), in particular to glycosyl hydrolase family 76.

Paragraph 32. The polypeptide of paragraph 31, which comprises one or more of the motifs [YND]DD[QINLEM] (SEQ ID NO: 60) and GG[ILMV]X[WS] (SEQ ID NO: 61).

Paragraph 33. The polypeptide of any of paragraphs 31-32, which further comprises the motif [RK][NLT]XXX[NTV]XP[GTLYISAVFNM] (SEQ ID NO: 64).

Paragraph 34. The polypeptide of any of paragraphs 31-33, which is of the KNTPA clade and is of bacterial origin.

Paragraph 35. The polypeptide of any of paragraphs 31-32, further comprising the motif GA]XX[AVL][ML]X[MA][ATV][EATV] (SEQ ID NO: 62) or the motif LA[EQ]X[VL][YF] (SEQ ID NO: 63).

Paragraph 36. The polypeptide of any of paragraphs 31-32 or 35, which is of the AMXAAE clade and is of fungal origin.

Paragraph 37. The polypeptide of paragraph 32, which comprises the motif N[EQD][WFY][HG]E (SEQ ID NO: 66).

Paragraph 38. The polypeptide of any preceding polypeptide paragraph, selected from the group consisting of:
(a) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 3 or a fragment thereof having alpha-mannan degrading activity;
(b) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 6 or a fragment thereof having alpha-mannan degrading activity;
(c) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 9 or a fragment thereof having alpha-mannan degrading activity;
(d) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 12 or a fragment thereof having alpha-mannan degrading activity;
(e) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 15 or a fragment thereof having alpha-mannan degrading activity;
(f) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 18 or a fragment thereof having alpha-mannan degrading activity;
(g) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 21 or a fragment thereof having alpha-mannan degrading activity;
(h) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 24 or a fragment thereof having alpha-mannan degrading activity;
(i) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 27 or a fragment thereof having alpha-mannan degrading activity;
(j) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 30 or a fragment thereof having alpha-mannan degrading activity;
(k) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 33 or a fragment thereof having alpha-mannan degrading activity;
(l) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 36 or a fragment thereof having alpha-mannan degrading activity;
(m) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 39 or a fragment thereof having alpha-mannan degrading activity;
(n) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 42 or a fragment thereof having alpha-mannan degrading activity;
(o) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 45 or a fragment thereof having alpha-mannan degrading activity;
(p) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 48 or a fragment thereof having alpha-mannan degrading activity;
(q) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 51 or a fragment thereof having alpha-mannan degrading activity;
(r) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 54 or a fragment thereof having alpha-mannan degrading activity; and
(s) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 57 or a fragment thereof having alpha-mannan degrading activity. and with the proviso that the isolated or purified polypeptide is not the polypeptide of geneseqp:AXR38305.

Paragraph 39. The polypeptide of any preceding polypeptide paragraph, selected from the group consisting of:
(a) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 3 or a fragment thereof having alpha-mannan degrading activity;
(b) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 6 or a fragment thereof having alpha-mannan degrading activity;
(c) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 9 or a fragment thereof having alpha-mannan degrading activity;
(d) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 12 or a fragment thereof having alpha-mannan degrading activity;
(e) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 15 or a fragment thereof having alpha-mannan degrading activity;
(f) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 18 or a fragment thereof having alpha-mannan degrading activity;
(g) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 21 or a fragment thereof having alpha-mannan degrading activity;
(h) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 24 or a fragment thereof having alpha-mannan degrading activity; and
(i) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 57 or a fragment thereof having alpha-mannan degrading activity. and with the proviso that the isolated or purified polypeptide is not the polypeptide of geneseqp:AXR38305.

Paragraph 40. The polypeptide of any preceding polypeptide paragraph, selected from the group consisting of:
(a) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 33 or a fragment thereof having alpha-mannan degrading activity;
(b) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 36 or a fragment thereof having alpha-mannan degrading activity;
(c) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 39 or a fragment thereof having alpha-mannan degrading activity;
(d) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 42 or a fragment thereof having alpha-mannan degrading activity;
(e) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 45 or a fragment thereof having alpha-mannan degrading activity;
(f) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 48 or a fragment thereof having alpha-mannan degrading activity;
(g) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 51 or a fragment thereof having alpha-mannan degrading activity;
(h) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 54 or a fragment thereof having alpha-mannan degrading activity.

Paragraph 41. An isolated or purified polypeptide having alpha-mannan degrading activity, selected from the group consisting of:
(a) a polypeptide having at least 60% sequence identity to the mature polypeptide of SEQ ID NO: 2, SEQ ID NO: 5, SEQ ID NO: 8, SEQ ID NO: 11, SEQ ID NO: 14, SEQ ID NO: 17, SEQ ID NO: 20, SEQ ID NO: 23, SEQ ID NO: 26, SEQ ID NO: 29, SEQ ID NO: 32, SEQ ID NO: 35, SEQ ID NO: 38, SEQ ID NO: 41, SEQ ID NO: 44, SEQ ID NO: 47, SEQ ID NO: 50, SEQ ID NO: 53, SEQ ID NO: 56;
(b) a polypeptide encoded by a polynucleotide that hybridizes under medium stringency conditions with the full-length complement of the mature polypeptide coding sequence of SEQ ID NO: 1, SEQ ID NO: 4, SEQ ID NO: 7, SEQ ID NO: 10, SEQ ID NO: 13, SEQ ID NO: 16, SEQ ID NO: 19, SEQ ID NO: 22, SEQ ID NO: 25, SEQ ID NO: 28, SEQ ID NO: 31 or the cDNA sequence thereof, SEQ ID NO: 34 or the cDNA sequence thereof, SEQ ID NO: 37 or the cDNA sequence thereof, SEQ ID NO: 40 or the cDNA sequence thereof, SEQ ID NO: 43 or the cDNA sequence thereof, SEQ ID NO: 46 or the cDNA sequence thereof, SEQ ID NO: 46 or the cDNA sequence thereof, SEQ ID NO: 49 or the cDNA sequence thereof, SEQ ID NO: 52 or the cDNA sequence thereof, or SEQ ID NO: 55;
(c) a polypeptide encoded by a polynucleotide having at least 60% sequence identity to the mature polypeptide coding sequence of SEQ ID NO: 1, SEQ ID NO: 4, SEQ ID NO: 7, SEQ ID NO: 10, SEQ ID NO: 13, SEQ ID NO: 16, SEQ ID NO: 19, SEQ ID NO: 22, SEQ ID NO: 25, SEQ ID NO: 28, SEQ ID NO: 31 or the cDNA sequence thereof, SEQ ID NO: 34 or the cDNA sequence thereof, SEQ ID NO: 37 or the cDNA sequence thereof, SEQ ID NO: 40 or the cDNA sequence thereof, SEQ ID NO: 43 or the cDNA sequence thereof, SEQ ID NO: 46 or the cDNA sequence thereof, SEQ ID NO: 46 or the cDNA sequence thereof, SEQ ID NO: 49 or the cDNA sequence thereof, SEQ ID NO: 52 or the cDNA sequence thereof, or SEQ ID NO: 55;
(d) a fragment of the polypeptide of (a), (b), or (c), that has alpha-mannan degrading activity; and with the proviso that the isolated or purified polypeptide is not the polypeptide of geneseqp:AXR38305.

Paragraph 42. The polypeptide of paragraph 35, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 2, SEQ ID NO: 5, SEQ ID NO: 8, SEQ ID NO: 11, SEQ ID NO: 14, SEQ ID NO: 17, SEQ ID NO: 20, SEQ ID NO: 23, SEQ ID NO: 26, SEQ ID NO: 29, SEQ ID NO: 32, SEQ ID NO: 35, SEQ ID NO: 38, SEQ ID NO: 41, SEQ ID NO: 44, SEQ ID NO: 47, SEQ ID NO: 50, SEQ ID NO: 53, SEQ ID NO: 56.

Paragraph 43. The polypeptide of paragraph 41 or 42, which is encoded by a polynucleotide that hybridizes under medium stringency conditions, medium-high stringency conditions, high stringency conditions, or very high stringency conditions with the full-length complement of the mature polypeptide coding sequence of SEQ ID NO: 1, SEQ ID NO: 4, SEQ ID NO: 7, SEQ ID NO: 10, SEQ ID NO: 13, SEQ ID NO: 16, SEQ ID NO: 19, SEQ ID NO: 22, SEQ ID NO: 25, SEQ ID NO: 28, SEQ ID NO: 31 or the cDNA sequence thereof, SEQ ID NO: 34 or the cDNA sequence thereof, SEQ ID NO: 37 or the cDNA sequence thereof, SEQ ID NO: 40 or the cDNA sequence thereof, SEQ ID NO: 43 or the cDNA sequence thereof, SEQ ID NO: 46 or the cDNA sequence thereof, SEQ ID NO: 49 or the cDNA sequence thereof, SEQ ID NO: 52 or the cDNA sequence thereof, or SEQ ID NO: 55.

Paragraph 44. The polypeptide of any one of paragraphs 41-43, which is encoded by a polynucleotide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide coding sequence of SEQ ID NO: 1, SEQ ID NO: 4, SEQ ID NO: 7, SEQ ID NO: 10, SEQ ID NO: 13, SEQ ID NO: 16, SEQ ID NO: 19, SEQ ID NO: 22, SEQ ID NO: 25, SEQ ID NO: 28, SEQ ID NO: 31 or the cDNA sequence thereof, SEQ ID NO: 34 or the cDNA sequence thereof, SEQ ID NO: 37 or the cDNA sequence thereof, SEQ ID NO: 40 or the cDNA sequence thereof, SEQ ID NO: 43 or the cDNA sequence thereof, SEQ ID NO: 46 or the cDNA sequence thereof, SEQ ID NO: 49 or the cDNA sequence thereof, SEQ ID NO: 52 or the cDNA sequence thereof, or SEQ ID NO: 55.

Paragraph 45. The polypeptide of any one of paragraphs 41-44, which is a variant of the mature polypeptide of SEQ ID NO: 2, SEQ ID NO: 5, SEQ ID NO: 8, SEQ ID NO: 11, SEQ ID NO: 14, SEQ ID NO: 17, SEQ ID NO: 20, SEQ ID NO: 23, SEQ ID NO: 26, SEQ ID NO: 29, SEQ ID NO: 32, SEQ ID NO: 35, SEQ ID NO: 38, SEQ ID NO: 41, SEQ ID NO: 44, SEQ ID NO: 47, SEQ ID NO: 50 SEQ ID NO: 53, or SEQ ID NO: 56, comprising a substitution, deletion, and/or insertion at one or more positions.

Paragraph 46. The polypeptide of any one of paragraphs 41-45, comprising, consisting essentially of, or consisting of SEQ ID NO: 2, SEQ ID NO: 5, SEQ ID NO: 8, SEQ ID NO: 11, SEQ ID NO: 14, SEQ ID NO: 17, SEQ ID NO: 20, SEQ ID NO: 23, SEQ ID NO: 26, SEQ ID NO: 29, SEQ ID NO: 32, SEQ ID NO: 35, SEQ ID NO: 38, SEQ ID NO: 41, SEQ ID NO: 44, SEQ ID NO: 47, SEQ ID NO: 50, SEQ ID NO: 53, or SEQ ID NO: 56, or the mature polypeptide thereof.

Paragraph 47. The polypeptide of any one of paragraphs 41-46, which is a fragment of SEQ ID NO: 2, SEQ ID NO: 5, SEQ ID NO: 8, SEQ ID NO: 11, SEQ ID NO: 14, SEQ ID NO: 17, SEQ ID NO: 20, SEQ ID NO: 23, SEQ ID NO: 26, SEQ ID NO: 29, SEQ ID NO: 32, SEQ ID NO: 35, SEQ ID NO: 38, SEQ ID NO: 41, SEQ ID NO: 44, SEQ ID NO: 47, SEQ ID NO: 50, SEQ ID NO: 53, or SEQ ID NO: 56 or the mature polypeptide thereof, wherein the fragment has alpha-mannan degrading activity.

Paragraph 48. A fusion polypeptide comprising the polypeptide of any one of paragraphs 41-47 and a second polypeptide.

Paragraph 49. A granule, which comprises: (a) a core comprising the polypeptide of any one of paragraphs 31-48, and, optionally (b) a coating consisting of one or more layer(s) surrounding the core.

Paragraph 50. A composition comprising the polypeptide of any one of paragraphs 31-48.

Paragraph 51. A liquid composition comprising a polyol and the polypeptide of any of paragraphs 31-48.

Paragraph 52. A whole broth formulation or cell culture composition comprising the polypeptide of any one of paragraphs 31-48.

Paragraph 53. An isolated or purified polynucleotide encoding the polypeptide of any one of paragraphs 31-48.

Paragraph 54. A nucleic acid construct or expression vector comprising the polynucleotide of paragraph 53, wherein the polynucleotide is preferably operably linked to one or more control sequences that direct the production of the polypeptide in an expression host.

Paragraph 55. A recombinant host cell comprising the polynucleotide of paragraph 53 operably linked to one or more control sequences that direct the production of the polypeptide.

Paragraph 56. The recombinant host cell of paragraph 55, wherein the polypeptide is heterologous to the recombinant host cell.

Paragraph 57. A method of producing the polypeptide of any one of paragraphs 31-48, comprising cultivating a cell, which in its wild-type form produces the polypeptide, under conditions conducive for production of the polypeptide.

Paragraph 52. The method of paragraph 51, further comprising recovering the polypeptide.

Paragraph 53. Use of the polypeptide of any one of paragraphs 31-48 for laundering, washing or cleaning a textile and/or a hard surface (such as dish wash).

Paragraph 54. Use of the polypeptide of any one of paragraphs 31-48 for
(a) preventing, reducing or removing stickiness of the item;
(b) preventing, reducing or removing biofilm or biofilm components;
(c) preventing, reducing or removing redeposition of soil during a wash cycle;
(d) preventing, reducing or removing adherence of soil to the item;
(e) maintaining or improving whiteness of the item; or
(f) preventing, reducing or removing malodor from the item,
wherein the item is a textile.

Paragraph 55. A process for degrading alpha-mannan, comprising applying the polypeptide of any one of paragraphs 31-48 to the alpha-mannan.

Paragraph 56. A method of cleaning an item, comprising the steps of:
(a) contacting the item with the polypeptide of any one of paragraphs 31-48; and
(b) and optionally rinsing the item, wherein the item is preferably a textile or a hard surface.

Paragraph 57. A process for preparing a food or feed composition and/or food or feed additive, comprising mixing the polypeptide of any of paragraphs 31-48, the granule of paragraph 49, the composition of paragraph 50, or the liquid composition of paragraph 51 with one or more food or feed and/or food or feed additive ingredients.

Paragraph 58. A process for producing a coffee extract, comprising the steps:
(a) providing roast and ground coffee beans;
(b) adding to said coffee beans water and a polypeptide of any of paragraphs 25-48, the granule of paragraph 49, the composition of paragraph 50, or the liquid composition of paragraph 51;
(c) incubating to make an aqueous coffee extract; and
(d) separating the coffee extract from the extracted coffee beans.

Paragraph 59. A process for degrading a cellulosic material, comprising: treating the cellulosic material with the polypeptide of any of paragraphs 25-48, the granule of paragraph 49, the composition of paragraph 50, or the liquid composition of paragraph 51.

Paragraph 60. A process for producing a fermentation product, comprising:
(a) saccharifying a cellulosic material with an enzyme composition in the presence of the polypeptide of any of paragraphs 25-48, the granule of paragraphs 49, the composition of paragraphs 50, or the liquid composition of paragraphs 51;
(b) fermenting the saccharified cellulosic material with one or more fermenting microorganisms to produce the fermentation product; and
(c) recovering the fermentation product from the fermentation.

The present invention is further described by the following examples that should not be construed as limiting the scope of the invention.

### EXAMPLES

### Materials

Chemicals used as buffers and substrates were commercial products of at least reagent grade.

### Model Detergents

Model detergent A wash liquor (100%) was prepared by dissolving 3.33 g/l of model detergent A containing 12% LAS, 11% AEO Biosoft N25-7 (NI), 17.63% AEOS (SLES), 6% MPG, 3% ethanol, 3.33% TEA (triethanolamine), 2.75% cocoa soap, 2.75% soya soap, 1.7% glycerol, 1.75% sodium hydroxide, 2% sodium citrate, 1% sodium formate, 0.48% DTMPA and 0.46% PCA (all percentages are w/w (weight volume) in water with hardness 15 dH.

Triple-20 Nonionic Model Detergent (60% surfactant) was prepared by dissolving 3.33 g/I non-ionic detergent containing NaOH 0.87%, MPG (Monopropylenglycol) 6%, Glycerol 2%, Soap-soy 2.75%, Soap-coco 2.75%, PCA (Sokalon CP-5) 0.2%, AEO Biosoft N25-7(NI) 16%, Sodium formiate 1%, Sodium Citrate 2%, DTMPA 0.2%, Ethanol (96%) 3%, adjustment of pH with NaOH or Citric acid ass water to 100% (all percentages are w/w (weight volume) in water with hardness 15 dH.

Model Detergent MC: A medical cleaning model detergent (model detergent MC) was prepared containing 5% MPG (propylene glycol), 5% Pluronic PE 4300 (PO/EO block polymer; 70%/30%, approx. 1750 g/mol), 2% Plurafac LF 305 (fatty alcohol alkoxylate; C6-10+EO/PO), 1% MGDA (methyl glycine diacetic acid, 1% TEA (triethanolamine) (all percentages are w/w). The pH was adjusted to 8.7 with phosphoric acid.

### Mini Launder-O-Meter (MiniLOM) Model Wash System

MiniLOM is a modified mini wash system of the Launder-O-Meter (LOM), which is a medium scale model wash system that can be applied to test up to 20 different wash conditions simultaneously. A LOM is basically a large temperature controlled water bath with 20 closed metal beakers rotating inside it. Each beaker constitutes one small washing machine and during an experiment, each will contain a solution of a specific detergent/enzyme system to be tested along with the soiled and unsoiled fabrics it is tested on. Mechanical stress is achieved by the beakers being rotated in the water bath and by including metal balls in the beaker.

The LOM model wash system is mainly used in medium scale testing of detergents and enzymes at European wash conditions. In a LOM experiment, factors such as the ballast to soil ratio and the fabric to wash liquor ratio can be varied. Therefore, the LOM provides the link between small scale experiments, such as AMSA and mini-wash, and the more time consuming full scale experiments in front loader washing machines. In miniLOM, washes are performed in 50 ml test tubes placed in Stuart rotator.

### Example 1: Reducing End Assay

For estimating the mannose yield after substrate hydrolysis, a reducing end assay developed by Lever (Anal. Biochem. 47: 273-279, 1972) is used. The assay is based on 4-hydroxybenzoic acid hydrazide, which under alkaline conditions reacts with the reducing ends of saccharides. The product is a strong yellow anion, which absorbs at 405 nm.

Method. The hydrolysis reaction mixture is composed of 20 μL enzyme and 180 μL substrate dissolved in buffer. The substrate is alfa-1,6-mannan prepared as described elsewhere (Cuskin, Nature, 2015, 517, 165-169) at a concentration of 2 mg/mL. The buffer is 25 mM acetate, pH5.5, 50 mM KCl, 0.01% Triton X-100, 1 mM CaCl2. The reaction conditions are 30 minutes, 37° C., and 950 rpm. 4-Hydroxybenzhydrazide (PAHBAH) (Sigma, H9882) is diluted in PAHBAH buffer to a concentration of 15 mg/ml. PAHBAH buffer contains: 50 g/L K—Na-tartrate (Merck, 1.08087) and 20 g/L sodium hydroxide (Sigma, S8045). This PAHBAH mix is made just before usage. 70 μL PAHBAH mix and MiliQ water are mixed in a 96 well PCR plate (Thermo Scientific). Samples from hydrolysis experiment are added. Samples and MiliQ always reached the total volume of 150 μL, but the dilution of the sample differed. The plate is sealed with Adhesive PCR Sealing Foil Sheets (Thermo Scientific). Plates are incubated at 95° C. for 10 min, cooled down and kept at 10° C. for 1 min in PTC-200 Thermal Cycler (MJ Research). 100 μL sample is transferred to a 96 well microtiter plate, flat bottomed (Nunc™) and color development measured at 405 nm on a SpectraMax 190 Absorbance Microplate Reader (Molecular Devices). Results are compared to mannose standards, which had undergone the same treatment and dilution as the samples to which they were compared.

### Example 1A: Beta-Mannanase Assay

For estimating the beta-mannanase activity, a reducing end assay developed by Lever (Anal. Biochem. 47: 273-279, 1972) is used. The assay is based on 4-hydroxybenzoic acid hydrazide, which under alkaline conditions reacts with the reducing ends of saccharides. The product is a strong yellow anion, which absorbs at 405 nm.

Method. The hydrolysis reaction mixture is composed of 20 μL enzyme and 180 μL substrate dissolved in buffer. The substrate is locust bean gum (5 g/L) and the buffer is 0.1 M Na-phosphate pH 7.5. The reaction conditions are 20 minutes, 37° C., and 950 rpm. 4-Hydroxybenzhydrazide (PAHBAH) (Sigma, H9882) is diluted in PAHBAH buffer to a concentration of 15 mg/ml. PAHBAH buffer contains: 50 g/L K—Na-tartrate (Merck, 1.08087) and 20 g/L sodium hydroxide (Sigma, S8045). This PAHBAH mix is made just before usage. 70 μL PAHBAH mix and MiliQ water are mixed in a 96 well PCR plate (Thermo Scientific). Samples from hydrolysis experiment are added. Samples and MiliQ always reached the total volume of 150 μL, but the dilution of the sample differed. The plate is sealed with Adhesive PCR Sealing Foil Sheets (Thermo Scientific). Plates are incubated at 95° C. for 10 min, cooled down and kept at 10° C. for 1 min in PTC-200 Thermal Cycler (MJ Research). 100 μL sample is transferred to a 96 well microtiter plate, flat bottomed (Nunc™) and color development measured at 405 nm on a SpectraMax 190 Absorbance Microplate Reader (Molecular Devices). Results are compared to mannose standards, which had undergone the same treatment and dilution as the samples to which they were compared.

### Example 2: Cloning and Expression of Alpha-Mannan Degrading Enzymes from Bacterial Strains

The alpha-mannan degrading enzymes were derived from bacterial strains isolated from environmental sample by standard microbiological isolation techniques. The isolated pure strains were identified, and taxonomy was assigned based on DNA sequencing of the 16S ribosomal genes (Table 1). One strain was purchased from the American Type Culture Collection as Bacillus circulans ATCC 21590 but assigned to the species Paenibacillus glycanilyticus based on the results of 16S ribosomal gene sequencing.

TABLE 1

| | Strain or community | Source Country | Mature protein |
|---|---|---|---|
| 1 | Paenibacillus | ATCC 21590 | SEQ ID NO: 3 |
| | glycanilyticus | | |
| 2 | Bacillus acidicola | Denmark | SEQ ID NO: 6 |
| 3 | Bacillus sp. | Denmark | SEQ ID NO: 9 |
| 4 | Paenibacillus sp. A | Denmark | SEQ ID NO: 12 |
| 5 | Paenibacillus sp. B | Denmark | SEQ ID NO: 15 |
| 6 | Paenibacillus sp. C | China | SEQ ID NO: 18 |
| 7 | Microbial community B | Denmark | SEQ ID NO: 21 |
| 8 | Microbial community H | Denmark | SEQ ID NO: 24 |
| 9 | Bacillus novalis | Netherlands | SEQ ID NO: 27 |
| 10 | Chryseobacterium sp. | United States | SEQ ID NO: 30 |
| | | of America | |
| 11 | Microbial community H | Denmark | SEQ ID NO: 57 |

### Preparation of Samples 1-6

Chromosomal DNA was isolated from pure cultures with the DNeasy Blood & Tissue Kit from Qiagen (Hilden, Germany) and subjected to full genome sequencing using Illumina technology. Genome sequencing, the subsequent assembly of reads and the gene discovery (i.e. annotation of gene functions) is known to the person skilled in the art and the service can be purchased commercially.

The genome sequences were analyzed for putative alpha mannanases from the CAZY database GH76 family (Lombard V, Golaconda Ramulu H, Drula E, Coutinho P M, Henrissat B (2014) The Carbohydrate-active enzymes database (CAZy) in 2013. Nucleic Acids Res 42:D490-D495.). This analysis identified 6 genes encoding putative GH76 mannanases which were subsequently cloned and recombinantly expressed in Bacillus subtilis.

The linear integration constructs were SOE-PCR fusion products (Horton, R. M., Hunt, H. D., Ho, S. N., Pullen, J. K. and Pease, L. R. (1989) Engineering hybrid genes without the use of restriction enzymes, gene splicing by overlap extension Gene 77: 61-68) made by fusion of the gene of interest between two B. subtilis chromosomal regions along with strong promoters and a chloramphenicol resistance marker. The SOE-PCR method is also described in patent application WO 2003095658.

The mannanase genes were expressed under the control of a triple promoter system (as described in WO 99/43835), consisting of the promoters from Bacillus licheniformis alpha-amylase gene (amyL), Bacillus amyloliquefaciens alpha-amylase gene (amyQ), and the Bacillus thuringiensis cryIIIA promoter including stabilizing sequence.

The genes were fused with DNA encoding a Bacillus clausii secretion signal (encoding the following amino acid sequence: MKKPLGKIVASTALLISVAFSSSIASA (SEQ ID NO: 58)) replacing the native secretion signal. Furthermore, the expression construct results in the addition of a amino-terminal poly histidine tag consisting of the amino acid sequence HHHHHHPR (SEQ ID NO: 59)) to the mature mannanase to facilitate easy purification by immobilized metal affinity chromatography.

The SOE-PCR product was transformed into Bacillus subtilis and integrated in the chromosome by homologous recombination into the pectate lyase locus. Subsequently a recombinant Bacillus subtilis clone containing the integrated expression construct was grown in liquid culture. The culture broth was centrifuged (20000×g, 20 min) and the supernatant was carefully decanted from the precipitate and used for purification of the enzyme or alternatively sterile filtered supernatant was used directly for assays.

### Purification of the Recombinant Enzyme by Immobilized Metal Affinity Chromatography

The pH of the cleared supernatant was adjusted to pH 8, filtrated through a 0.2 μM filter, and the supernatant applied to a 5 ml HisTrap™ excel column. Prior to loading, the column had been equilibrated in 5 column volumes (CV) of 50 mM Tris/HCl pH 8. In order to remove unbound material, the column was washed with 8 CV of 50 mM Tris/HCl pH 8, and elution of the target was obtained with 50 mM HEPES pH 7+10 mM imidazole. The eluted protein was desalted on a HiPrep™ 26/10 desalting column, equilibrated using 3 CV of 50 mM HEPES pH 7+100 mM NaCl. This buffer was also used for elution of the target, and the flow rate was 10 ml/min. Relevant fractions were selected and pooled based on the chromatogram and SDS-PAGE analysis.

### Preparation of Samples 7-11:

Strains are cloned and expressed according to the above protocol

### Example 3: Cloning and Expression of Alpha-Mannan Degrading Enzymes from Aspergillus aculeatus

The alpha mannan degrading enzyme was derived from Aspergillus aculeatus strain CBS172.66 isolated from a tropical soil sample in 1962.

Genomic DNA was isolated from Aspergillus aculeatus strain CBS172.66 using a FastDNA spin for Soil Kit (MP Biomedicals, OH, USA).

The Joint Genome Institute Aspergillus aculeatus ATCC16872 genome assembly v1 sequences were analyzed for putative alpha mannanases from the CAZY database GH76 family (Lombard V, Golaconda Ramulu H, Drula E, Coutinho PM, Henrissat B (2014) The Carbohydrate-active enzymes database (CAZy) in 2013. Nucleic Acids Res 42:D490-D495). This analysis identified a gene encoding a putative GH76 mannanase which was subsequently cloned and recombinantly expressed in Aspergillus oryzae.

The gene encoding the putative GH76 mannanase was cloned by PCR amplification from genomic DNA using gene-specific primers that also append a Kozak translation initiation sequence “TCCACC” immediately 5′ of the start codon. The amplified DNA fragment was cloned into cloned into the expression vector pDAu222 as described in WO 2013024021 using BamHI and XhoI restriction sites.

The sequence of the putative GH76 mannanase encoding gene cloned in the expression vector was confirmed and the expression construct was transformed into the Aspergillus oryzae strain MT3568 (WO 11/057140) to produce the secreted mature peptide with protein sequence SEQ ID NO: 33. Transformants were selected on acetamide during regeneration from protoplasts and subsequently re-isolated under selection (Christensen et al., 1988, Biotechnology 6, 1419-1422 and WO 04/032648).

For production of the recombinant mannanase, a single Aspergillus transformant was cultured in twenty 500 ml baffled flasks each containing 150 ml of DAP-4C-1 medium (WO 12/103350). The cultures were shaken on a rotary table at 100 RPM at 30° C. for 4 days. The culture broth was subsequently separated from cellular material by passage through a 0.22 μm filter.

Purification of the recombinant Aspergillus aculeatus mannanase Filtrated broth was adjusted to pH7.0 and filtrated on 0.22 μm PES filter (Nalge Nunc International, Nalgene labware cat #595-4520). Following, the filtrate was added 1.8M ammonium sulphate. The filtrate was loaded onto a Phenyl Sepharose™ 6 Fast Flow column (high sub) (GE Healthcare, Piscataway, NJ, USA) equilibrated with 1.8M ammonium sulphate, 25 mM HEPES pH7.0. The bound protein was eluted with 1.0M ammonium sulphate, 25 mM HEPES pH 7.0. Fractions were collected and analyzed by SDS-PAGE. The fractions were pooled and applied to a Sephadex™ G-25 (medium) (GE Healthcare, Piscataway, NJ, USA) column equilibrated in 25 mM HEPES pH 7.0. The fractions were applied to a SOURCE™ 15Q (GE Healthcare, Piscataway, NJ, USA) column equilibrated in 25 mM HEPES pH 7.0 and bound proteins were eluted with a linear gradient from 0-1000 mM sodium chloride over 20CV. Fractions were collected and analyzed by SDS-PAGE.

### Example 4. Cloning, Expression and Purification of Alpha-Mannan Degrading Enzymes from Fungal Strains

Seven genes encoding alpha-mannanases belonging to the CAZy defined protein family GH76 (www.cazy.org, Lombard V, et al. (2014) Nucleic Acids Res 42:D490-D495) were cloned from two fungal strains that were isolated from environmental samples and are described in Table 2 below. The Aspergillus aculeatus isolate is available from CBS-KNAW Fungal Biodiversity Centre, Utrecht, The Netherlands, as CBS 101.43.

TABLE 2

| | | Mature peptide |
|---|---|---|
| | | sequence |
| Strain | Source country | SEQ ID NO: |
| Aspergillus aculeatus | Unknown, isolated | SEQ ID NO: 36 |
| | on or prior to 1943. | SEQ ID NO: 39 |
| | | SEQ ID NO: 42 |
| Humicola insolens | Denmark | SEQ ID NO: 45 |
| | | SEQ ID NO: 48 |
| | | SEQ ID NO: 51 |
| | | SEQ ID NO: 54 |

Chromosomal DNA was isolated and used for whole genome sequencing by standard methods known to the person skilled in the art. The whole genome sequences were assembled with either IDBA or SPAdes genome assemblers (Peng, Y., et al. Bioinformatics. (2012), 28: 1420-1428 and Bankevich, A. et al. J Comput Biol. (2012) 19(5):455-77), and genes were annotated on the genomes with the GeneMark 2.3c or GeneMark ES v4.28 gene prediction software (Ter-Hovhannisyan V. et al. Genome Res. (2008) 18(12):1979-90.).

The set of peptide sequences predicted from genes annotated on the whole genome sequences were searched for similarity to the GH76 domain. Seven peptides identified in this search are listed in the table above with SEQ ID NOs for both the nucleotide and peptide sequences. The genes encoding these peptides were cloned by PCR amplification from genomic DNA using gene-specific primers that also append a Kozak translation initiation sequence “TCACC” immediately 5′ of the start codon. The predicted peptides for six of the alpha-manannases (corresponding to SEQ ID NO: 39, SEQ ID NO: 42, SEQ ID NO: 45, SEQ ID NO: 48, SEQ ID NO: 51, SEQ ID NO: 54) included a predicted GPI anchor or transmembrane domain at the C-terminus. To promote secretion of the recombinant proteins and avoid GPI or transmembrane domain mediated anchoring to the cell membrane, PCR primers were designed to amplify truncated peptides that included the full coding region for the predicted GH76 catalytic domain but did not include the C-terminal GPI or transmembrane domain. Stop codons were added in frame via the return PCR primer. The truncated nucleotide sequences amplified for expression are shown in SEQ ID NO: 39, SEQ ID NO: 42, SEQ ID NO: 45, SEQ ID NO: 48, SEQ ID NO: 51, SEQ ID NO: 54, respectively. The amplified DNA fragments were cloned into the Aspergillus expression vector pMStr57 (WO 04/032648) that had been digested with BamHI and XhoI.

The cloned genes were sequenced and confirmed to be identical to the corresponding genes found in the genome sequence, and transformed into the Aspergillus oryzae strain MT3568 (WO 11/057140) by the methods described in Christensen et al., 1988, Biotechnology 6, 1419-1422 and WO 04/032648. Transformants were selected during regeneration from protoplasts based on the ability, conferred by a selectable marker in the expression vector, to utilize acetamide as a nitrogen source, and were subsequently re-isolated twice under selection.

Production of the recombinant polypeptides was evaluated by culturing transformants in 96-well deep-well microtiter plates for 4 days at 30° C. in 0.25 ml of YPG medium (WO 05/066338) or DAP-4C-1 medium (WO 12/103350) and monitoring recombinant expression by SDS-PAGE.

Chromatographic Purification of Seven Family GH76 Alpha-Mannanases from Aspergillus aculeatus and Humicola insolens

For larger-scale production of the recombinant alpha-mannanases, a single Aspergillus transformant was selected for each mannanase based on recombinant yield, the transformants were cultured in 500 ml baffled flasks containing 150 ml of DAP-4C-1 medium. The cultures were shaken on a rotary table at 150 RPM at a temperature of 30° C. for 4 days. Culture broth was separated from cellular material by passage through a 0.22 um filtration unit.

To purify the samples, the pH of the filtered samples was adjusted to around pH 7.5 and 1.8M ammonium sulfate (AMS) was added. The samples were applied to a 5 ml HiTrap™ Phenyl (HS) column on an Äkta Explorer. Prior to loading, the column had been equilibrated in 5 column volumes (CV) of 50 mM HEPES+1.8M AMS pH 7. In order to remove unbound material, the column was washed with 5 CV of 50 mM HEPES+1.8M AMS pH 7. The target proteins were eluted from the column into a 10 ml loop using 50 mM HEPES+20% isopropanol pH 7. From the loop, the samples were loaded onto a desalting column (HiPrep™ 26/10 Desalting), which had been equilibrated with 3CV of 50 mM HEPES+100 mM NaCl pH 7.0. The target proteins were eluted with 50 mM HEPES+100 mM NaCl pH 7.0 and relevant fractions were selected and pooled based on the chromatograms. The flow rate was 5 ml/min.

Protein concentration in the final samples were estimated by measuring absorption at 280 nm.

### Example 5: Milliscale Wash Assay (MISWA96)

### Preparation of Biofilm Swatches

Biofilm swatches (10 cm×10 cm) were made by growing Brevundimonas sp. on polyester swatches for three days. The biofilm swatches were rinsed twice in water and dried for 2 h in a laf bench with flow and subsequently punched into round swatches circles (0.6 cm×0.6 cm) and placed into the wells of a MTP96, and stored at 4° C. for further use.

### Washing Experiment

The swatches were transferred from the MTP96 to a deep-well MTP96, for the washing assay. The deep-well plate was placed in a Hamilton robot and subjected to a wash simulation program using the following conditions: shaking speed: 30 sec at 1000 rpm. Duration of wash cycle: 30 minutes with shaking; temperature 30° C.; Volume of wash liquor (total): 500 μl per well (490 μl wash liquor of Model Detergent A+10 μl sample). For screening of wash performance of alpha-mannan degrading enzymes, wash liquor Model Detergent A was prepared by dissolving 3.3 g/L in water hardness 15° dH. Soil was subsequently added to reach a concentration of 0.7 g soil/L (WFK 09V pigment soil).

A 96 deep-well plate was filled with each enzyme sample, and the program was started on the robot. Alpha-mannan degrading enzymes were tested in concentration 0.5 ppm. The blank consisted of biofilm swatches without any enzyme addition. After completion of the wash simulation cycle, the swatches were removed from the wash liquor and dried on a filter paper. The dried swatches were fixed on a sheet of white paper for scanning. The scanned picture was further used with the software color-analyzer. Each sample have an intensity measurement from the color analyzer software analysis that will be used to calculate the delta intensity by subtracting the intensity of the blank without enzyme. Values over 20 delta intensity represents a visual cleaning effect. These data are shown in Table 3.

TABLE 3
Wash performance of alpha-mannan degrading enzymes.

| | | Intensity | | |
|---|---|---|---|---|
| | | (with alpha- | | |
| | | mannan | Intensity | |
| SEQ ID | | degrading | (without | Delta |
| NO: | Family | enzyme) | enzyme) | Intensity |
| 33 | GH76 | 202 | 180 | 22 |
| 36 | GH76 | 209 | 178 | 31 |
| 45 | GH76 | 205 | 178 | 27 |
| 48 | GH76 | 228 | 180 | 48 |
| 39 | GH76 | 215 | 178 | 37 |
| 51 | GH76 | 194 | 178 | 16 |
| 21 | GH76 | 252 | 212 | 57 |
| 24 | GH76 | 228 | 228 | 40 |
| 3 | GH76 | 228 | 228 | 36 |
| 6 | GH76 | 228 | 228 | 29 |
| 9 | GH76 | 228 | 228 | 9 |
| 12 | GH76 | 228 | 228 | 19 |
| 15 | GH76 | 228 | 228 | 28 |
| 18 | GH76 | 228 | 228 | 23 |
| 27 | GH92 | 217 | 206 | 11 |
| 30 | GH99 | 290 | 260 | 31 |

The combined effect of separate different alpha-mannan degrading enzymes was also tested, as described above, but with each alpha-mannan degrading enzyme tested in concentration 0.5 ppm (total enzyme concentration 1 ppm). These data are shown in Table 4.

TABLE 4
Wash performance of alpha-mannan degrading enzymes.

| | | Intensity | | |
|---|---|---|---|---|
| | | (when combined | Intensity | |
| SEQ ID | | with GH99 | (without | Delta |
| NO: | Family | SEQ ID NO: 30) | enzyme) | Intensity |
| 33 | GH76 | 284 | 179 | 105 |
| 36 | GH76 | 282 | 178 | 104 |
| 45 | GH76 | 264 | 178 | 86 |
| 48 | GH76 | 280 | 179 | 101 |
| 42 | GH76 | 285 | 178 | 107 |
| 39 | GH76 | 275 | 178 | 97 |
| 51 | GH76 | 261 | 178 | 83 |
| 54 | GH76 | 250 | 178 | 72 |
| 21 | GH76 | 327 | 181 | 146 |
| 27 | GH92 | 278.5 | 181 | 97.5 |

### Example 6: Construction of Clades and Phylogenetic Trees

### GH76 Phylogenetic Tree

A phylogenetic tree was constructed, of polypeptide sequences containing a GH76 domain, as defined in CAZY (GH76, Glycoside Hydrolase Family 76, CAZy database, www.cazy.org, Lombard V, et al. 2014, Nucleic Acids Res 42:D490-D495). The phylogenetic tree was constructed from a multiple alignment of mature polypeptide sequences containing at least one GH76 domain. The sequences were aligned using the MUSCLE algorithm version 3.8.31 (Edgar, 2004. Nucleic Acids Research 32(5): 1792-1797), and the trees were constructed using FastTree version 2.1.8 (Price et al., 2010, PloS one 5(3)) and visualized using iTOL (Letunic & Bork, 2007. Bioinformatics 23(1): 127-128).

The polypeptide containing a GH76 domain comprises several motifs. One example is [YND]DD[QINLEM] (SEQ ID NO: 60) situated in positions 124 to 127 in Paenibacillus glycanilyticus (SEQ ID NO: 3), where D at position 124 is the catalytic nucleophile of the family, and D in position 125 is the general acid/base. and fully conserved in the polypeptides of the invention. Another motif which may be comprised by the polypeptides of the invention is GG[ILMV]X[WS](SEQ ID NO: 61) situated in positions corresponding to positions 167 to 171 in Paenibacillus glycanilyticus (SEQ ID NO: 3).

The polypeptides comprising a GH76 domain can be further separated into multiple distinct sub-clusters, or clades, where the denoted clades are listed below.

### The AMXAAE Clade (GH76 Fungal)

The polypeptides containing a GH76 domain can be separated into distinct sub-clusters. The sub-clusters are defined by one or more short sequence motifs, as well as containing a GH76 domain as defined in CAZY (GH76, CAZy database, www.cazy.org, Lombard V, et al. 2014, Nucleic Acids Res 42:D490-D495).

We denoted one sub-cluster comprising the motif [GA]XX[AVL][ML]X[MA][ATV][EATV] (SEQ ID NO: 62) as the AMXAAE clade. It is situated at positions 99 to 107 in Aspergillus aculeatus (SEQ ID NO: 36). All polypeptide sequences containing a GH76 domain as well as the motif will be denoted as belonging to the AMXAAE clade. The AMXAAE clade comprises polypeptides of primarily fungal origin.

Another motif which may be comprised by members of the AMXAAE clade is LA[EQ]X[VL][YF] (SEQ ID NO: 63) situated in positions corresponding to positions 122 to 127 in Aspergillus aculeatus (SEQ ID NO: 36).

Examples of polypeptides of the AMXAAE clade includes SEQ ID NO: 33, SEQ ID NO: 36, SEQ ID NO: 39, SEQ ID NO: 42, SEQ ID NO: 45, SEQ ID NO: 48, SEQ ID NO: 51, and SEQ ID NO: 54.

### The KNTPA Clade (GH76 Bacterial)

The polypeptides containing a GH76 domain can be separated into additional distinct sub-clusters, using the phylogenetic tree described above. One clade is denote KNTPA, which comprises polypeptides of bacterial origin.

Members of the KNTPA clade must be of bacterial origin and contain the motif [RK][NLT]XXX[NTV]XP[GTLYISAVFNM] (SEQ ID NO: 64), corresponding to amino acids KNTPANAPA (SEQ ID NO: 65) at positions 147 to 155 in SEQ ID NO 3 from Paenibacillus glycanilyticus.

Examples of polypeptides of the KNTPA clade includes SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, and SEQ ID NO: 57.

### GH99 Phylogenetic Tree and the NEWHE Clade

A phylogenetic tree was constructed, of polypeptide sequences containing a GH99 domain, as defined in CAZY (GH99, Glycoside Hydrolase Family 99, CAZy database, www.cazy.org, Lombard V, et al. 2014, Nucleic Acids Res 42:D490-D495). The phylogenetic tree was constructed from a multiple alignment of mature polypeptide sequences containing at least one GH92 domain. The sequences were aligned using the MUSCLE algorithm version 3.8.31 (Edgar, 2004. Nucleic Acids Research 32(5): 1792-1797), and the trees were constructed using FastTree version 2.1.8 (Price et al., 2010, PloS one 5(3)) and visualized using iTOL (Letunic & Bork, 2007. Bioinformatics 23(1): 127-128).

The polypeptide containing a GH99 domain comprises several motifs. One example is N[EQD][WFY][HG]E (SEQ ID NO: 66) situated in positions 296 to 300 in Chryseobacterium sp. (SEQ ID NO: 30), where E at position 322 is the general acid/base of the family and fully conserved in the family.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

1. A cleaning composition comprising:
(a) at least one polypeptide having alpha-mannan degrading activity or a variant or a fragment thereof having alpha-mannan degrading activity; and
(b) at least one cleaning component, preferably selected from a surfactant, a builder, a bleach component, a polymer, a dispersing agent and/or an additional enzyme.

2. The cleaning composition of claim 1, wherein the polypeptide has alpha-mannanase and/or alpha-mannosidase activity.

3. The cleaning composition of claim 1, wherein the polypeptide having alpha-mannan degrading activity belongs to glycosyl hydrolase family 76 (GH76), glycosyl hydrolase family 92 (GH92), glycosyl hydrolase family 99 (GH99), in particular to glycosyl hydrolase family 76.

4. The cleaning composition of claim 1, wherein the polypeptide comprises one or more of the motifs [YND]DD[QINLEM] (SEQ ID NO: 60) and GG[ILMV]X[WS] (SEQ ID NO: 61).

5. The cleaning composition of claim 4, wherein the polypeptide further comprises the motif [RK] [NLT]XXX[NTV]XP[GTLYISAVFNM] (SEQ ID NO: 64).

6. The cleaning composition claim 1, wherein the polypeptide is of the KNTPA clade and is of bacterial origin.

7. The cleaning composition of claim 4, wherein the polypeptide further comprises the motif GA]XX[AVL][ML]X[MA][ATV][EATV] (SEQ ID NO: 62) or the motif LA[EQ]X[VL][YF] (SEQ ID NO: 63).

8. The cleaning composition of claim 1, wherein the polypeptide is of the AMXAAE clade and is of fungal origin.

9. The cleaning composition of claim 1, wherein the polypeptide comprises the motif N[EQD][WFY][HG]E (SEQ ID NO: 66).

10. The cleaning composition of claim 1, comprising at least two polypeptides having alpha-mannan degrading activity.

11. The cleaning composition of claim 1, comprising a polypeptide belonging to GH family 76 and a polypeptide belonging to GH family 99; a polypeptide belonging to GH family 76 and a polypeptide belonging to GH family 92; a polypeptide belonging to GH family 92 and a polypeptide belonging to GH family 99; in particular, a polypeptide belong to GH family 76 and a polypeptide belonging to GH family 99.

12. The cleaning composition of claim 1, wherein the polypeptide having alpha-mannan degrading activity is obtained from a strain of Bacillus, e.g., Bacillus acidicola, Bacillus novalis, or Bacillus sp., a strain of Paenibacillus, e.g., Paenibacillus glycanilyticus, or Paenibacillus sp., or from a strain of Aspergillus, e.g., Aspergillus aculeatus, or from a strain of Humicola, e.g., Humicola insolens.

13. The cleaning composition of claim 1, wherein the polypeptide is selected from the group consisting of:
(a) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 24;
(b) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 3;
(c) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 6;
(d) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 9;
(e) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 12;
(f) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 15;
(g) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 18;
(h) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 21;
(i) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 27;
(j) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 30;
(k) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 33;
(l) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 36;
(m) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 39;
(n) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 42;
(o) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 45;
(p) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 48;
(q) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 51;
(r) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 54, and;
(s) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 57.

14. The cleaning composition of claim 1, comprising at least 0.001 ppm of the polypeptide having alpha-mannan degrading activity.

15. A process for degrading alpha-mannan, comprising applying the cleaning composition of claim 1 to the alpha-mannan.

16. A method of cleaning an item, comprising the steps of:
(a) contacting the item with a cleaning composition claim 1; and
(b) and optionally rinsing the item, wherein the item is preferably a textile or a hard surface.

17. An isolated or purified polypeptide having alpha-mannan degrading activity, wherein the polypeptide having alpha-mannan degrading activity belongs to glycosyl hydrolase family 76 (GH76), glycosyl hydrolase family 92 (GH92), glycosyl hydrolase family 99 (GH99), in particular to glycosyl hydrolase family 76.

18. The polypeptide of claim 17, which comprises one or more of the motifs [YND]DD[QINLEM] (SEQ ID NO: 60) and GG[ILMV]X[WS] (SEQ ID NO: 61).

19. The polypeptide of claim 18, which further comprises the motif [RK][NLT]XXX[NTV]XP[GTLYISAVFNM] (SEQ ID NO: 64).

20. The polypeptide of claim 17, which is of the KNTPA clade and is of bacterial origin.

21. The polypeptide of claim 18, further comprising the motif GA]XX[AVL][ML]X[MA][ATV][EATV] (SEQ ID NO: 62) or the motif LA[EQ]X[VL][YF] (SEQ ID NO: 63).

22. The polypeptide of claim 17, which is of the AMXAAE clade and is of fungal origin.

23. The polypeptide of claim 17, which comprises the motif N[EQD][WFY][HG]E (SEQ ID NO: 66).

24. The polypeptide of claim 17, selected from the group consisting of:
(a) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 24 or a fragment thereof having alpha-mannan degrading activity;
(b) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 3 or a fragment thereof having alpha-mannan degrading activity;
(c) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 6 or a fragment thereof having alpha-mannan degrading activity;
(d) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 9 or a fragment thereof having alpha-mannan degrading activity;
(e) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 12 or a fragment thereof having alpha-mannan degrading activity;
(f) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 15 or a fragment thereof having alpha-mannan degrading activity;
(g) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 18 or a fragment thereof having alpha-mannan degrading activity;
(h) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 21 or a fragment thereof having alpha-mannan degrading activity;
(i) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 27 or a fragment thereof having alpha-mannan degrading activity;
(j) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 30 or a fragment thereof having alpha-mannan degrading activity;
(k) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 33 or a fragment thereof having alpha-mannan degrading activity;
(l) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 36 or a fragment thereof having alpha-mannan degrading activity;
(m) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 39 or a fragment thereof having alpha-mannan degrading activity;
(n) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 42 or a fragment thereof having alpha-mannan degrading activity;
(o) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 45 or a fragment thereof having alpha-mannan degrading activity;
(p) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 48 or a fragment thereof having alpha-mannan degrading activity;
(q) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 51 or a fragment thereof having alpha-mannan degrading activity;
(r) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 54 or a fragment thereof having alpha-mannan degrading activity; and
(s) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 57 or a fragment thereof having alpha-mannan degrading activity.

25. An isolated or purified polypeptide having alpha-mannan degrading activity, selected from the group consisting of:
(a) a polypeptide having at least 60% sequence identity to the mature polypeptide of SEQ ID NO: 2, SEQ ID NO: 5, SEQ ID NO: 8, SEQ ID NO: 11, SEQ ID NO: 14, SEQ ID NO: 17, SEQ ID NO: 20, SEQ ID NO: 23, SEQ ID NO: 26, SEQ ID NO: 29, SEQ ID NO: 32, SEQ ID NO: 35, SEQ ID NO: 38, SEQ ID NO: 41, SEQ ID NO: 44, SEQ ID NO: 47, SEQ ID NO: 50, SEQ ID NO: 53, SEQ ID NO: 56;
(b) a polypeptide encoded by a polynucleotide that hybridizes under medium stringency conditions with the full-length complement of the mature polypeptide coding sequence of SEQ ID NO: 1, SEQ ID NO: 4, SEQ ID NO: 7, SEQ ID NO: 10, SEQ ID NO: 13, SEQ ID NO: 16, SEQ ID NO: 19, SEQ ID NO: 22, SEQ ID NO: 25, SEQ ID NO: 28, SEQ ID NO: 31 or the cDNA sequence thereof, SEQ ID NO: 34 or the cDNA sequence thereof, SEQ ID NO: 37 or the cDNA sequence thereof, SEQ ID NO: 40 or the cDNA sequence thereof, SEQ ID NO: 43 or the cDNA sequence thereof, SEQ ID NO: 46 or the cDNA sequence thereof, SEQ ID NO: 46 or the cDNA sequence thereof, SEQ ID NO: 49 or the cDNA sequence thereof, SEQ ID NO: 52 or the cDNA sequence thereof, or SEQ ID NO: 55;
(c) a polypeptide encoded by a polynucleotide having at least 60% sequence identity to the mature polypeptide coding sequence of SEQ ID NO: 1, SEQ ID NO: 4, SEQ ID NO: 7, SEQ ID NO: 10, SEQ ID NO: 13, SEQ ID NO: 16, SEQ ID NO: 19, SEQ ID NO: 22, SEQ ID NO: 25, SEQ ID NO: 28, SEQ ID NO: 31 or the cDNA sequence thereof, SEQ ID NO: 34 or the cDNA sequence thereof, SEQ ID NO: 37 or the cDNA sequence thereof, SEQ ID NO: 40 or the cDNA sequence thereof, SEQ ID NO: 43 or the cDNA sequence thereof, SEQ ID NO: 46 or the cDNA sequence thereof, SEQ ID NO: 46 or the cDNA sequence thereof, SEQ ID NO: 49 or the cDNA sequence thereof, SEQ ID NO: 52 or the cDNA sequence thereof, or SEQ ID NO: 55;
(d) a fragment of the polypeptide of (a), (b), or (c), that has alpha-mannan degrading activity.


SEQUENCE LISTING (69 sequences)

SEQ ID NO: 1 - DNA, 1158 bp, Paenibacillus glycanilyticus
  sig_peptide 1..96
  mat_peptide 97..1155
  source 1..1158: mol_type = genomic DNA
  CDS 1..1155
ttgaatcgcaacaaacgatatttatggctggccgttgcttcggcagttatcctcatagcgttggtctggtcattggtctggaaggataaaacggcagcaacctccgtcatttggcaggaacgggcggaagaagcgcagcttgaactgaccaagtccttctgggacgacaagcgcgggctctacaacaatgccgcgccgtgtattgcccagctatgcaccgatccgtttaactattggtggctggcgcatgcggtggatgcgctggtcgacggctacgagcgcagcggggatgaacggtacgcggagcaaatcgccaagctgcatcaaggattgcttgatcgcaacgccggcgtgatgatcaacgattattacgatgatatggaatggatggcgctggcctggctgagagcttacgatgccaccaaggacgagaagtataagcaggaagcacttgaactgtgggaagagattaaaggcggctggaacgaggagatgggcggcggcatcgcttggcgcaaggagcagctcgattacaagaatacgccggcgaatgctcctgcggctattttggcggcacggctgtacggccatttccataacggggaagatctggcctgggcgaaaaaaatttacgattggcaaaaagagacgcttgtggatccggataccggtctcgtatgggacggaatcaaccggacgggcgacggcaatatcgacaaggaatggcgattcacgtatggccaaggcgtgtttatcggggccggcgttgaactgttccgggcaacggaggataaggcttatctggaggatgcccgccggacggcggctcatctgaaggaggcgttcctgtctccggcaaccggcatgcttccatcggaaggcgacggcgacgggggcttgttcaaaggcgtattaatccgctatttgggagagctgattgccgctgatccggatgagccggagcgcaaagaatggattggcatgcttgagaccaacgcgaacagcttatggcaatacggaaaagcggaagataaagcggtattcagcaattcatgggccgaagcgccggatacgatcgtgcaattaagcacggaactaagcgggataatgctgctcgagcagatggccgtattggagaaaaacggacaattacagtag

SEQ ID NO: 2 - protein, 385 aa, Paenibacillus glycanilyticus
LNRNKRYLWLAVASAVILIALVWSLVWKDKTAATSVIWQERAEEAQLELTKSFWDDKRGLYNNAAPCIAQLCTDPFNYWWLAHAVDALVDGYERSGDERYAEQIAKLHQGLLDRNAGVMINDYYDDMEWMALAWLRAYDATKDEKYKQEALELWEEIKGGWNEEMGGGIAWRKEQLDYKNTPANAPAAILAARLYGHFHNGEDLAWAKKIYDWQKETLVDPDTGLVWDGINRTGDGNIDKEWRFTYGQGVFIGAGVELFRATEDKAYLEDARRTAAHLKEAFLSPATGMLPSEGDGDGGLFKGVLIRYLGELIAADPDEPERKEWIGMLETNANSLWQYGKAEDKAVFSNSWAEAPDTIVQLSTELSGIMLLEQMAVLEKNGQLQ

SEQ ID NO: 3 - protein, 353 aa, Paenibacillus glycanilyticus
ATSVIWQERAEEAQLELTKSFWDDKRGLYNNAAPCIAQLCTDPFNYWWLAHAVDALVDGYERSGDERYAEQIAKLHQGLLDRNAGVMINDYYDDMEWMALAWLRAYDATKDEKYKQEALELWEEIKGGWNEEMGGGIAWRKEQLDYKNTPANAPAAILAARLYGHFHNGEDLAWAKKIYDWQKETLVDPDTGLVWDGINRTGDGNIDKEWRFTYGQGVFIGAGVELFRATEDKAYLEDARRTAAHLKEAFLSPATGMLPSEGDGDGGLFKGVLIRYLGELIAADPDEPERKEWIGMLETNANSLWQYGKAEDKAVFSNSWAEAPDTIVQLSTELSGIMLLEQMAVLEKNGQLQ

SEQ ID NO: 4 - DNA, 1137 bp, Bacillus acidicola
  sig_peptide 1..87
  mat_peptide 88..1134
  source 1..1137: mol_type = genomic DNA
  CDS 1..1134
atgtctaaatttcttaaaccaagtattgtgtttactctgattgtgatgctaatcgcgttagtggcacctggcacgtctacatatgctgcatcctatcataagaaaaatgttgaaagagcccttgcctcttatgaattaatgcaaaagtacttctatcagcctagtgttaaactttatactgaagaatttcctaacgttatcgggaattcatattcttatttatggccgttttcccaagcaatggctgcaacaactgatgtgtctagactccctaaaataggtaaaaattatgtatttgatagaaatgacaggctggatggactgaaattgtactggaataatggaacaaaccctgccggttacgattcctatgttcgtcctcctcttggtcagggcggggataagttttatgatgataatgattggattgcccttaatctcatcaaattgtatcaattaactggtgatcaggctgtattagaacgggtcaaggatattttcaaattggaagtgtacggatgggacaatgatccatcacacccttatcgaggaggaatattttggacgcaagcatcctggagtcaggatcggaatacgatttccaacgcacctttggcacaaatcgggctttatttataccaaattacccgagataaatcttattttgactgggcaaaaaaagcctacgactgggtaaataattctatgctggcaccaaacggtttatattgggatcatgttgatcttaaaggaaatattgataaaactcagtggacttacaatcaaggaatgatgattggggcaaacgtattgttctataaaacaacaggcaatgaaacatatttagatcttgcaaaatctattgcaaataaagctatccaatattatggcgatgttcttctttataataatcctccagaattcaacgcaatcttctttgaaaacttacagctattggattccatcaatcataacaatatatatcgaaaatatatacagtcgtatgctgatcaaatgtgggatacagaaagaaatacggaaacaggcttatttcagcgtgataatcaaaatccagtgcctctaattgagcaagcagcaatagttgaaatttattctaatttggcttatcaaaaataa

SEQ ID NO: 5 - protein, 378 aa, Bacillus acidicola
MSKFLKPSIVFTLIVMLIALVAPGTSTYAASYHKKNVERALASYELMQKYFYQPSVKLYTEEFPNVIGNSYSYLWPFSQAMAATTDVSRLPKIGKNYVFDRNDRLDGLKLYWNNGTNPAGYDSYVRPPLGQGGDKFYDDNDWIALNLIKLYQLTGDQAVLERVKDIFKLEVYGWDNDPSHPYRGGIFWTQASWSQDRNTISNAPLAQIGLYLYQITRDKSYFDWAKKAYDWVNNSMLAPNGLYWDHVDLKGNIDKTQWTYNQGMMIGANVLFYKTTGNETYLDLAKSIANKAIQYYGDVLLYNNPPEFNAIFFENLQLLDSINHNNIYRKYIQSYADQMWDTERNTETGLFQRDNQNPVPLIEQAAIVEIYSNLAYQK

SEQ ID NO: 6 - protein, 349 aa, Bacillus acidicola
ASYHKKNVERALASYELMQKYFYQPSVKLYTEEFPNVIGNSYSYLWPFSQAMAATTDVSRLPKIGKNYVFDRNDRLDGLKLYWNNGTNPAGYDSYVRPPLGQGGDKFYDDNDWIALNLIKLYQLTGDQAVLERVKDIFKLEVYGWDNDPSHPYRGGIFWTQASWSQDRNTISNAPLAQIGLYLYQITRDKSYFDWAKKAYDWVNNSMLAPNGLYWDHVDLKGNIDKTQWTYNQGMMIGANVLFYKTTGNETYLDLAKSIANKAIQYYGDVLLYNNPPEFNAIFFENLQLLDSINHNNIYRKYIQSYADQMWDTERNTETGLFQRDNQNPVPLIEQAAIVEIYSNLAYQK

SEQ ID NO: 7 - DNA, 1143 bp, Bacillus sp.
  sig_peptide 1..90
  mat_peptide 91..1140
  source 1..1143: mol_type = genomic DNA
  CDS 1..1140
atgcgtaaaaaatttcttaaaacatgtttggtacttactctggttgtcatggcagcgttatggatggttcctggtacgccaacacttgcagcgtcccatcaaaagaaaaatacgcttagatccataaccacttatgaatcacttcaaaagtacttctatcagccaagtgcaaagctttataccgaagaatatcccagggagggagggaatccatattcctatgtgtggcctttttcccgggcaatggctgccacgattgatatgacaaggattccgaaaatagggaaagcatacgtatcggatcgaaacgaacggctcgagggactgaagctgtattggaataatgaaaccaaccctgccggctacgattcatatgttcgtcctcctgtaggccagggcggggacaaattctatgatgacaatgattggattgcccttaacttcctcaaactgtatcaattaacaggtgaccattccgcattaaagcgggccaaggatatctttaagctggaggtgcttgggtgggacaatgatccatcgcatccatatccaggaggagtattctggacgcaggcaccgtggagccaggaccggaataccatttccaatgcgcctgtggcacagattgggttatatctataccaaatcactggtgataaatactattttgactgggcaaagaaaacctatgactgggtaaacaattctatgctcgcgccaaacggcttgtattgggaccatgtcgatctaaaagggaatatagagaaaactcagtggacctataaccagggaatgatgatcggggcaaacgttcttttctataaaacaacaggtgacaaaatgtatcttgaacgtgcagagtcccttgcagaaaagtctatccaatattatggcgacacccagctgtataaaaatcctccagaattcaacgccatcttcttcgaaaacctgcagctattgtactcagtcaaacataataatgaatatagaaaatatactcaagcctatgcggataaaatgtgggatacggtgcgaaatccgaaaacgggcttattccaacgtgatcctcaaaagccggtgccgcttatagagcaggctgcgatggttgagatttatgcgaatctagcatataaaaaacaataa

SEQ ID NO: 8 - protein, 380 aa, Bacillus sp.
MRKKFLKTCLVLTLVVMAALWMVPGTPTLAASHQKKNTLRSITTYESLQKYFYQPSAKLYTEEYPREGGNPYSYVWPFSRAMAATIDMTRIPKIGKAYVSDRNERLEGLKLYWNNETNPAGYDSYVRPPVGQGGDKFYDDNDWIALNFLKLYQLTGDHSALKRAKDIFKLEVLGWDNDPSHPYPGGVFWTQAPWSQDRNTISNAPVAQIGLYLYQITGDKYYFDWAKKTYDWVNNSMLAPNGLYWDHVDLKGNIEKTQWTYNQGMMIGANVLFYKTTGDKMYLERAESLAEKSIQYYGDTQLYKNPPEFNAIFFENLQLLYSVKHNNEYRKYTQAYADKMWDTVRNPKTGLFQRDPQKPVPLIEQAAMVEIYANLAYKKQ

SEQ ID NO: 9 - protein, 350 aa, Bacillus sp.
ASHQKKNTLRSITTYESLQKYFYQPSAKLYTEEYPREGGNPYSYVWPFSRAMAATIDMTRIPKIGKAYVSDRNERLEGLKLYWNNETNPAGYDSYVRPPVGQGGDKFYDDNDWIALNFLKLYQLTGDHSALKRAKDIFKLEVLGWDNDPSHPYPGGVFWTQAPWSQDRNTISNAPVAQIGLYLYQITGDKYYFDWAKKTYDWVNNSMLAPNGLYWDHVDLKGNIEKTQWTYNQGMMIGANVLFYKTTGDKMYLERAESLAEKSIQYYGDTQLYKNPPEFNAIFFENLQLLYSVKHNNEYRKYTQAYADKMWDTVRNPKTGLFQRDPQKPVPLIEQAAMVEIYANLAYKKQ

SEQ ID NO: 10 - DNA, 1533 bp, Paenibacillus sp.
  sig_peptide 1..93
  mat_peptide 94..1530
  source 1..1533: mol_type = genomic DNA
  CDS 1..1530
atggcaaagttcggcaagaaagcatctgctatcgccagtggtattttggctgctgcacttctgattgcaccctttgggggagaacaacgtgcagctgcgtttaccgcagcgaatgccgacacggcgatgaacagttttgtcagtaccttttatgatcctgtagcgaaatatttctataccaacagcgaccatctgattcattccgagcatgcgcatggacctgacggtggtctgtataccgacttctggtgggaggcccagctctgggagacggtgatggatgcgtatgagcggactggcagcagcacctaccgtgccatgatcgatgatgtttataccggatttaatgcgaaatacccggatatgatggccaatgattttaatgatgatctcggctggtgggcgctcgcctgtatgcgggcttatgaactgactggcaccgatgagtaccgcaaccgtgcgtcgttcctgttcgaccagatctggggcgattgggacagtacgtatggcggcggtatctggtggaagcgggatggcacttcgccgcagaagaatatggcgaccaatgcgccgatggttatgacggctattaagctgaagaatgccacgggcaacaacgattatttgacaaaagcgcagagtatctacagctggattcagagcagactggtctccggcagcaaaatcaatgaccacgtcgaaggctccggctccgggacggtggtagattgggactttacctataattacggcacgtatctcggcgcagcactggccctgaatcaagctacggggaatgcatcctatctcacggatgcgaacacagccgcagcttatgtcatggataaaatgacgctctcctattcgcttatgtacgaaggagagaatgattcgcctggcttccgcatggtgttcgcccgcaatttgaaccagctgcggaaggcgacagggaacgcttcctacttgaatttcttgcaacagaatgctacgcaagcctttaaccaccgccgtgcgtctgacgggattattgacagcgactggacagcgcctgccagaagcggctatatccagtctatcgccgcagccgcaggtgcttcgatcctgcaactggtccctgcggacaattacaccggaccgattgccggcaacggtacctatgaggctgagaacgcccgccgctacggcatcaacaatgaatcctcgcagccaggcttctcgggacgggggtatacagcaggctggaacaccgataatacgaaaattgtgttccatgttaatcagaacagtgccagtacaaggaccattagctttcgttataccgctgcgggggggaatgctggacggtacgtcaaagtgaacggtacggtggtcagcaataatttgatttttaacggtaccagcagttggtctgcctggaacacggtgacgctaaacgtaccgcttaatgcaggctacaactcgattgagctcggatttgatagcaccaagggcaacagcaattatctgaatgtagacaaaatgacgggactttaa

SEQ ID NO: 11 - protein, 510 aa, Paenibacillus sp.
MAKFGKKASAIASGILAAALLIAPFGGEQRAAAFTAANADTAMNSFVSTFYDPVAKYFYTNSDHLIHSEHAHGPDGGLYTDFWWEAQLWETVMDAYERTGSSTYRAMIDDVYTGFNAKYPDMMANDFNDDLGWWALACMRAYELTGTDEYRNRASFLFDQIWGDWDSTYGGGIWWKRDGTSPQKNMATNAPMVMTAIKLKNATGNNDYLTKAQSIYSWIQSRLVSGSKINDHVEGSGSGTVVDWDFTYNYGTYLGAALALNQATGNASYLTDANTAAAYVMDKMTLSYSLMYEGENDSPGFRMVFARNLNQLRKATGNASYLNFLQQNATQAFNHRRASDGIIDSDWTAPARSGYIQSIAAAAGASILQLVPADNYTGPIAGNGTYEAENARRYGINNESSQPGFSGRGYTAGWNTDNTKIVFHVNQNSASTRTISFRYTAAGGNAGRYVKVNGTVVSNNLIFNGTSSWSAWNTVTLNVPLNAGYNSIELGFDSTKGNSNYLNVDKMTGL

SEQ ID NO: 12 - protein, 479 aa, Paenibacillus sp. A1
AAFTAANADTAMNSFVSTFYDPVAKYFYTNSDHLIHSEHAHGPDGGLYTDFWWEAQLWETVMDAYERTGSSTYRAMIDDVYTGFNAKYPDMMANDFNDDLGWWALACMRAYELTGTDEYRNRASFLFDQIWGDWDSTYGGGIWWKRDGTSPQKNMATNAPMVMTAIKLKNATGNNDYLTKAQSIYSWIQSRLVSGSKINDHVEGSGSGTVVDWDFTYNYGTYLGAALALNQATGNASYLTDANTAAAYVMDKMTLSYSLMYEGENDSPGFRMVFARNLNQLRKATGNASYLNFLQQNATQAFNHRRASDGIIDSDWTAPARSGYIQSIAAAAGASILQLVPADNYTGPIAGNGTYEAENARRYGINNESSQPGFSGRGYTAGWNTDNTKIVFHVNQNSASTRTISFRYTAAGGNAGRYVKVNGTVVSNNLIFNGTSSWSAWNTVTLNVPLNAGYNSIELGFDSTKGNSNYLNVDKMTGL

SEQ ID NO: 13 - DNA, 1563 bp, Paenibacillus sp. B
  sig_peptide 1..93
  mat_peptide 94..1560
  source 1..1563: mol_type = genomic DNA
  CDS 1..1560
atgattgtctggaaaaaacgtgtgaccctcgctatggcttttctgcttgtcttgtccctgctgggcccgaccggcagccgaaaagcggaagcgttcaccgcagccaatgccaatgatgctatgcaagccttcattaacgttttctatgacccgacggccaagtatttctacacgaacagtgaccaccagattcacacgcacgcgcatggtccgaacggcgggctgtataccgacttctggtgggaggctcagttgtgggagacggtaatggatgcctatgagcgaacaggcaacgccacttaccgcaccatgattgatgatatttacaccggcttcaatgccaagtacccggatatgatgcagaatgtgttcaacgacgacctcggctggtgggcgcaggctgccctgcgggcctatgagctgacggggacagcggagtatcgcaaccggggctcatttctcttcgacaagatctatgaggaatgggacacgagctactacggcggcggcatctggtggcggagagatgctcataacccgaatgtctcgtccaatgcccagaagaacgtagctacgaacgcaccaatggtcattacagctgtgaagctgtaccaggcgacgggcgacagcgcttatctgacgaaggcgacgcagatctacaactgggtcaaaaccaagctcgtcgggtcaggcggcaagatcaatgaccatctggaaggtccgggggcaggcacgctcatcgactgggacttctcgtacaactacggcaactacctgggcgcggcggtgtcgctgtatcaggcaacgggcaacagtgcctacatcaccgatgccaacacagccgcgacctatgccattaataacctggtgtcggcgcagacccttatgtatgagggtgagaatgatgccgccggcttcaaaatgatcttcgctcgcaacctgaaccggctgcgcgtcatcggtggccagtcgcagtatctgaacttcttgcagcagaacgcgacacaggcctggaatcaccgccgcaccagcgataatatcatcggcagcgactggctccggcctaccggctccggttatatccagagtctagccgcagcggcgggtgtctcgatcctgcagctgacgccgccagacaactatacgggttacatcgccggcaacggtgcctacgaggctgagaacgcccagcggacgctggtgagcggcgggggcatgatcaacgagagcacgcagggcggatatacgggccgcggctatgtcgcgggctggaatacgaccggcacttcgctcaacttctacgtgaaccagaacaccgccggcaatcgcaccatcaccttccgctatgcggcgggtgcgggcaacgcctcgcggtatgtacgtgtgaacggcgtctatgtggccaacaatctgagcttcagcggcacctcaggctggggcagctggaacaccgtctccgtcacagtcccgctgaacgccggctcgaacacgatccagctcggctacgacagttcccgaggcaatagtaacttcttgaacgtagatattctgagcggtttgtaa

SEQ ID NO: 14 - protein, 520 aa, Paenibacillus sp. B
MIVWKKRVTLAMAFLLVLSLLGPTGSRKAEAFTAANANDAMQAFINVFYDPTAKYFYTNSDHQIHTHAHGPNGGLYTDFWWEAQLWETVMDAYERTGNATYRTMIDDIYTGFNAKYPDMMQNVFNDDLGWWAQAALRAYELTGTAEYRNRGSFLFDKIYEEWDTSYYGGGIWWRRDAHNPNVSSNAQKNVATNAPMVITAVKLYQATGDSAYLTKATQIYNWVKTKLVGSGGKINDHLEGPGAGTLIDWDFSYNYGNYLGAAVSLYQATGNSAYITDANTAATYAINNLVSAQTLMYEGENDAAGFKMIFARNLNRLRVIGGQSQYLNFLQQNATQAWNHRRTSDNIIGSDWLRPTGSGYIQSLAAAAGVSILQLTPPDNYTGYIAGNGAYEAENAQRTLVSGGGMINESTQGGYTGRGYVAGWNTTGTSLNFYVNQNTAGNRTITFRYAAGAGNASRYVRVNGVYVANNLSFSGTSGWGSWNTVSVTVPLNAGSNTIQLGYDSSRGNSNFLNVDILSGL

SEQ ID NO: 15 - protein, 489 aa, Paenibacillus sp. B
FTAANANDAMQAFINVFYDPTAKYFYTNSDHQIHTHAHGPNGGLYTDFWWEAQLWETVMDAYERTGNATYRTMIDDIYTGFNAKYPDMMQNVFNDDLGWWAQAALRAYELTGTAEYRNRGSFLFDKIYEEWDTSYYGGGIWWRRDAHNPNVSSNAQKNVATNAPMVITAVKLYQATGDSAYLTKATQIYNWVKTKLVGSGGKINDHLEGPGAGTLIDWDFSYNYGNYLGAAVSLYQATGNSAYITDANTAATYAINNLVSAQTLMYEGENDAAGFKMIFARNLNRLRVIGGQSQYLNFLQQNATQAWNHRRTSDNIIGSDWLRPTGSGYIQSLAAAAGVSILQLTPPDNYTGYIAGNGAYEAENAQRTLVSGGGMINESTQGGYTGRGYVAGWNTTGTSLNFYVNQNTAGNRTITFRYAAGAGNASRYVRVNGVYVANNLSFSGTSGWGSWNTVSVTVPLNAGSNTIQLGYDSSRGNSNFLNVDILSGL

SEQ ID NO: 16 - DNA, 1110 bp, Paenibacillus sp. C
  sig_peptide 1..63
  mat_peptide 64..1107
  source 1..1110: mol_type = genomic DNA
  CDS 1..1107
atgaaaagcatatgcatgctgcttgtcggcagttttttcttgacaggctgcaatggaaatgaggagcgcttggaaaattacaataatcatgcggaatgggcagaagagaagctcatcgagcattattggaatgagaatgggaagctgatgaacaacgcttatccttatagcaaagaacgcgaggagagcctaaactattggtggaaggcgcatgccgtggatgccatgatggatgggtatgagagaacgggtgatgctgcttatacggatagggcagaggggatcgtcaaaagcattattaaaaaaaatgggtcattgtttaatgaattttatgatgatatggaatggctcgctcttgcagcgctgaggctatatgatgcaaccaaaagcgagacggttaaaggttatgttttaaagctgtggaatgatattaagaccgcttggtgggaagatgagctgggcggcatggcttggaagaaggatcaacgagacagccggaatgcatgctcgaacgggccggcagctattttggctgccaggatgtatgagtattttggcgataaggaagatctggaatgggccaaaaaaatatatgattggcagaagaaaaatttggttgatcctgaaagcggtcttgtgtatgatggccttaagctggaagagaatggtgatcttaatgttaataaaaattggatattcacctataaccaaggcacttatataggcgcagcagttgagctttataagcatacaaatgacaagacgtatttggcagatgcggagaaaaccgctgcctcggctatgaaatatcatacggttgctgagaccggaatgatgaaggaagaaggaaccggagacggtggactgttcaaaggcatattcattcgttatctgatccatttgtatgaagtcaacgaaagcgtccaaattaaagaaatgatctttcataatgcggatacactgctctcgaaaggcagcacgaaggaaattggcttgtttggtcctgcttgggatatgcctcatcaggagcctctggatatatccgtgcaattgagcggagttttcttaatagaagccgcagccaaaatagaaagactagattcggaataa

SEQ ID NO: 17 - protein, 369 aa, Paenibacillus sp. C
MKSICMLLVGSFFLTGCNGNEERLENYNNHAEWAEEKLIEHYWNENGKLMNNAYPYSKEREESLNYWWKAHAVDAMMDGYERTGDAAYTDRAEGIVKSIIKKNGSLFNEFYDDMEWLALAALRLYDATKSETVKGYVLKLWNDIKTAWWEDELGGMAWKKDQRDSRNACSNGPAAILAARMYEYFGDKEDLEWAKKIYDWQKKNLVDPESGLVYDGLKLEENGDLNVNKNWIFTYNQGTYIGAAVELYKHTNDKTYLADAEKTAASAMKYHTVAETGMMKEEGTGDGGLFKGIFIRYLIHLYEVNESVQIKEMIFHNADTLLSKGSTKEIGLFGPAWDMPHQEPLDISVQLSGVFLIEAAAKIERLDSE

SEQ ID NO: 18 - protein, 348 aa, Paenibacillus sp. C
ERLENYNNHAEWAEEKLIEHYWNENGKLMNNAYPYSKEREESLNYWWKAHAVDAMMDGYERTGDAAYTDRAEGIVKSIIKKNGSLFNEFYDDMEWLALAALRLYDATKSETVKGYVLKLWNDIKTAWWEDELGGMAWKKDQRDSRNACSNGPAAILAARMYEYFGDKEDLEWAKKIYDWQKKNLVDPESGLVYDGLKLEENGDLNVNKNWIFTYNQGTYIGAAVELYKHTNDKTYLADAEKTAASAMKYHTVAETGMMKEEGTGDGGLFKGIFIRYLIHLYEVNESVQIKEMIFHNADTLLSKGSTKEIGLFGPAWDMPHQEPLDISVQLSGVFLIEAAAKIERLDSE

SEQ ID NO: 19 - DNA, 1719 bp, synthetic construct
  misc_feature 1..1719: note = Synthetic construct
  sig_peptide 1..105
  mat_peptide 106..1719
  source 1..1719: mol_type = other DNA
  CDS 1..1719
atgtcacttagatcaggtcaacttttcaggcttctggcggtgcctctggctatcgcacttatgctcggttccatgccgggcatcggatcgtccaaagcctacgcctataccgcatcggacggggatactgcgatgagagcttttaacgatacattctgggacccgaacgccaagatgttctggaaggactcgaagcgagaaaagcatcaagacttctgggtggaggcagagctgtgggaattggtcatggatgcatatcagcatacctccgatcctgccttgaaagccgagcttaaaacgcagatcgacgatgtatatgacggcaccgtcgccaagtacggccaagattggacgaataatcccttcaatgacgatattatgtggtgggcgatgggaagcgccagagcctatcaaatcaccgggaatccaagatatttgaaggccgcaaaggatcatttcgattttgtgtacgatacgcagtgggatgaagagttcgcgagcggcggcatttggtggctgaacagcgaccataataccaagaatgcgtgcattaacttcccggcggcggaagctgcactttatctttacgatattacaaaagatgagcattatttgaacacggcaaccaaaatattcagatggggcaaaacgatgcttacggacggaaacggaaaagtgttcgaccgtatcgaagttgaacatggcgcggttccggatgccactcactacaaccagggtacatacatcggagcggccgtcggattgtatgaagcgaccggaaatgccgtttacctcgatgacgcggtcaaggccgctaaattcaccaagaatcacctggtggattcaaacggggtattgaattacgaaggccccaacggagatctgaaaggcggcaaaacgatcctgatgcgcaatctggcctatctgcaaaagacactggatgaaaccggccagcaccccgaattcagcgctgaattcgacgaatggcttgcattcaatgtcgaaatggcctggagccaccgcaatccggatcatatcgtggatggaaactgggcaggacagctgctgtccgggacctatgaatcctggtcctcggccgctgccgttcaagctttaaacgttatcaagccgatggaagcggagcttcgttatgccgtcaaaaacccctttgagaaaatcgaagcggaacgctacaacattggggccggattcgtgctggagggttcatttgaaggttcactgcaattaggcggaatccagcatggctcttatgccgcttacaaaaatgtggatttcggatcagacggcgcgatcggcttcattgccagagcgtctagcggaacgggcggaggccaaattgagatccggcttgattccaaggatggccccaaagtcgggaccttgaacgtagagggaacgggcgattggaatcattatatcgatgccgtcaccctccttaaagatgaccagggagcgccaagcaccataaccggcgtccatgatgtgtatcttgtcttcaccaagacaaatgacgattatttattcaatttaaactgggttcaatttacgacgacggacccgaccgaaaccgacgcctatgccaagctgaaggccggaaactatgacagcagcgaaggacttagtaaacatgccgagttcggatatttggacggaatccatcacaatgcctatgcctcttatgaaggaattgattttgga

SEQ ID NO: 20 - protein, 573 aa, synthetic construct
  REGION 1..573: note = Synthetic Construct
MSLRSGQLFRLLAVPLAIALMLGSMPGIGSSKAYAYTASDGDTAMRAFNDTFWDPNAKMFWKDSKREKHQDFWVEAELWELVMDAYQHTSDPALKAELKTQIDDVYDGTVAKYGQDWTNNPFNDDIMWWAMGSARAYQITGNPRYLKAAKDHFDFVYDTQWDEEFASGGIWWLNSDHNTKNACINFPAAEAALYLYDITKDEHYLNTATKIFRWGKTMLTDGNGKVFDRIEVEHGAVPDATHYNQGTYIGAAVGLYEATGNAVYLDDAVKAAKFTKNHLVDSNGVLNYEGPNGDLKGGKTILMRNLAYLQKTLDETGQHPEFSAEFDEWLAFNVEMAWSHRNPDHIVDGNWAGQLLSGTYESWSSAAAVQALNVIKPMEAELRYAVKNPFEKIEAERYNIGAGFVLEGSFEGSLQLGGIQHGSYAAYKNVDFGSDGAIGFIARASSGTGGGQIEIRLDSKDGPKVGTLNVEGTGDWNHYIDAVTLLKDDQGAPSTITGVHDVYLVFTKTNDDYLFNLNWVQFTTTDPTETDAYAKLKAGNYDSSEGLSKHAEFGYLDGIHHNAYASYEGIDFG

SEQ ID NO: 21 - protein, 538 aa, synthetic construct
  REGION 1..538: note = Synthetic construct
YTASDGDTAMRAFNDTFWDPNAKMFWKDSKREKHQDFWVEAELWELVMDAYQHTSDPALKAELKTQIDDVYDGTVAKYGQDWTNNPFNDDIMWWAMGSARAYQITGNPRYLKAAKDHFDFVYDTQWDEEFASGGIWWLNSDHNTKNACINFPAAEAALYLYDITKDEHYLNTATKIFRWGKTMLTDGNGKVFDRIEVEHGAVPDATHYNQGTYIGAAVGLYEATGNAVYLDDAVKAAKFTKNHLVDSNGVLNYEGPNGDLKGGKTILMRNLAYLQKTLDETGQHPEFSAEFDEWLAFNVEMAWSHRNPDHIVDGNWAGQLLSGTYESWSSAAAVQALNVIKPMEAELRYAVKNPFEKIEAERYNIGAGFVLEGSFEGSLQLGGIQHGSYAAYKNVDFGSDGAIGFIARASSGTGGGQIEIRLDSKDGPKVGTLNVEGTGDWNHYIDAVTLLKDDQGAPSTITGVHDVYLVFTKTNDDYLFNLNWVQFTTTDPTETDAYAKLKAGNYDSSEGLSKHAEFGYLDGIHHNAYASYEGIDFG

SEQ ID NO: 22 - DNA, 1731 bp, synthetic construct
  misc_feature 1..1731: note = Synthetic construct
  sig_peptide 1..105
  mat_peptide 106..1731
  source 1..1731: mol_type = other DNA
  CDS 1..1731
ttggccagtaaggttagaagaagattcaagcttatcgtattgcttgttgttcttgcactctcgatcagtaccctatctccaagcggaatgcctcgcgcccatgcttttgaagcggaggatgcgaagacggcaatcgttgcctacaatgatgcattctgggatgcaaacgcgaaatacttctggaaatccaccaatcgtaccgattatcaggacttttggatagaggccgagctttgggagctcgttatggatgcctatctgcatacatcggacccggagctcaaggcgcagctgcggacccagattgacgatgtgttcgatggagccgtaacgaggtacggcgaggactggacatataatccgtataacgatgacatcatgtggtgggcaatggcaagtgcaagagcctatcagattacgaatgacgaacggtatttggaacaagcggaatattatttcaattatgtctatgataatgaatgggatactgaatttgccggaggcggcatttggtggaaaagcgatgatcgcacgacaaaaaatgcatgcatcaactttcctgccgctcaaaccgctgtctttttgtataacgttacccaagacgaacaatacctggatgcagccgaaaccatctaccattggggaaaaacaatactgactgacggcaacggcaaagtattcgatcgaattgaaacgcaaaatggagctattcaaggcgcgactcactataatcaaggtgcgtttattgggtcagccgcaggtctctacgagattacgggagacacggattatctggacgatgcgatcaaagcggccacgtatacgaaggagcatatggttgacgtcaacggactgctgcgatatgagggccctaacggagatttgaagggcggcaaaacgattctgctgcgcaacctcggctattttcaagcggcgatagacgctcgccaggaagaaaattaccaatccttcgccgaaagctacaatgagtggctggcttttaatgccgatatggcatggaacaatcgcaatgcggccaatctcgtcgacggcaactgggcgggacaacagttatccggcgccatcgagtcatggagcgcggcagcggccgtacaagctttgatatccctaaagccgcagaacgcggtacagctgggatatgccgttaagaacccttataacaggatcgaagcggaaagttacaatattataaacggccctggcctggaggacagcaatgaaggctcgcaacaactggccggtattcaggacagtcattatgcggcatataaaaatgtcgattttggttcggaagatggtgcaagcggtttcattgcgcgagcatcgagcggaacgggcggcggccagatcgaaatcagactggatgctttggacggaccaaaggcaggtacgctaaatgtaaatggtaccggaggctggaacaactacatcgacgcggctgtactgctgaaggatgagcaagggaatccgagtcctgtgaccggcgtacacgatgtgtatctggtgtttaagaggacaaatgacacatatctattcaatctcaattggttccaatttacgaaggtcgatccgaccctgatttctgcttatacgatattgcaggccgagcattttgcaagcagtgacgggctgagtattaactcaacagggcaatatgcagacggcattcaaaataccgcatatgcttcctacgaaaatattgattttggc

SEQ ID NO: 23 - protein, 577 aa, synthetic construct
  REGION 1..577: note = Synthetic Construct
LASKVRRRFKLIVLLVVLALSISTLSPSGMPRAHAFEAEDAKTAIVAYNDAFWDANAKYFWKSTNRTDYQDFWIEAELWELVMDAYLHTSDPELKAQLRTQIDDVFDGAVTRYGEDWTYNPYNDDIMWWAMASARAYQITNDERYLEQAEYYFNYVYDNEWDTEFAGGGIWWKSDDRTTKNACINFPAAQTAVFLYNVTQDEQYLDAAETIYHWGKTILTDGNGKVFDRIETQNGAIQGATHYNQGAFIGSAAGLYEITGDTDYLDDAIKAATYTKEHMVDVNGLLRYEGPNGDLKGGKTILLRNLGYFQAAIDARQEENYQSFAESYNEWLAFNADMAWNNRNAANLVDGNWAGQQLSGAIESWSAAAAVQALISLKPQNAVQLGYAVKNPYNRIEAESYNIINGPGLEDSNEGSQQLAGIQDSHYAAYKNVDFGSEDGASGFIARASSGTGGGQIEIRLDALDGPKAGTLNVNGTGGWNNYIDAAVLLKDEQGNPSPVTGVHDVYLVFKRTNDTYLFNLNWFQFTKVDPTLISAYTILQAEHFASSDGLSINSTGQYADGIQNTAYASYENIDFG

SEQ ID NO: 24 - protein, 542 aa, synthetic construct
  REGION 1..542: note = Synthetic construct
FEAEDAKTAIVAYNDAFWDANAKYFWKSTNRTDYQDFWIEAELWELVMDAYLHTSDPELKAQLRTQIDDVFDGAVTRYGEDWTYNPYNDDIMWWAMASARAYQITNDERYLEQAEYYFNYVYDNEWDTEFAGGGIWWKSDDRTTKNACINFPAAQTAVFLYNVTQDEQYLDAAETIYHWGKTILTDGNGKVFDRIETQNGAIQGATHYNQGAFIGSAAGLYEITGDTDYLDDAIKAATYTKEHMVDVNGLLRYEGPNGDLKGGKTILLRNLGYFQAAIDARQEENYQSFAESYNEWLAFNADMAWNNRNAANLVDGNWAGQQLSGAIESWSAAAAVQALISLKPQNAVQLGYAVKNPYNRIEAESYNIINGPGLEDSNEGSQQLAGIQDSHYAAYKNVDFGSEDGASGFIARASSGTGGGQIEIRLDALDGPKAGTLNVNGTGGWNNYIDAAVLLKDEQGNPSPVTGVHDVYLVFKRTNDTYLFNLNWFQFTKVDPTLISAYTILQAEHFASSDGLSINSTGQYADGIQNTAYASYENIDFG

SEQ ID NO: 25 - DNA, 4236 bp, Bacillus novalis
  sig_peptide 1..99
  mat_peptide 100..4233
  source 1..4236: mol_type = genomic DNA
  CDS 1..4233
atgaaaaaaagagcgatccaaaagatcatttcgagtattgtatccctcgcagttctgaccagtgtccctgttatgcaaaaaccatctgtattggcagcatcgtctgattccacttcagcatcgaaaacggatttctttagctcgtttgaaaaatccgatctgcaactcacttggactaatacagtggagacagatgctaacggaaagaaaatgtcctcaggaattgatgggaatgttaagcgggatttaatccttggcgatattaccgataaagttgttcaagtaacagcgagtgcaaacaatcccccgaatgaaatcgattctaagttgatcgatggggacccgacaacaaaatggttagcatttgaaccgactgctaacatcgtccttaagctggctgaaccagtggccgttgtcaaatatgccttaacatctgctaatgacgctaaaggaagggatccaaaaaactggacattatacggttccttagatggaacgaattggacagccgtcgatacacgagaaggggaagattttaaagatcgcttccaacgaaatatgtatgatttaaaaaatactacaaagtacttgtattacaaattggacattacgaaaaatgctggtgacagtattacccagttagcggaaatttctttatcagacggaattgaagttccagcaccaccgccgggagatatgaagtcgctaataggaaaaggcccaactagttcttataccgctaaaacaaatgtgggctggaccggattaggtgcccttaattattcaggtacacatctttcagacggaagggcttattcttacaacaagctttatgatgtcgacatccttgttaccccagcaacagagctttcgtattttattgccccagaatttacagataaaaatcataatgactattcaagcacatatgtctctgtggaccttgccttttcagatggaacctaccttcatgatcttaaagcagtagaccagtatggtgtaggacttaatccgaaagaccaaggtgattcaaaatacttatacgtaaaccaatggaacacaattaaatctactattggttctgttgcggcaggtaaaaccataaaaaggattctagtagcctacgataatccaaaaggccctggagcctttagaggaagcatcgatgatattaaaattgatggaaaacctgttcagaaagcatttggttctccaattgactatgtaaatatccttcgcgggacacaatcgaacggatcattttctcgtggaaataacttcccagccgtggcgattccgcatggatttaacttttggacaccaactactaatgcaggatctagttggatttatcaatatcatgaaagcaacagtgtcaataatctcccacaaatccaagctttttctgtcagccatgaaccaagcccttggatgggggatcgtcagacattccaagtgatgccatccgcttcgacggcagctacaccaaatgcaaatcgtgattcacgtgcattagaatttaaccatgcgaatgaaattgcccagccacactattacagcgtgaaatttgaaaatgggattcgtacggaaatgactccaacagaccatgcagctatgtttaagtttacttttacaggtgccacatctaacctcatttttgataatgttaacaataatggaggccttacaatcgatgctaaatcaggagagatcaccggctattctgatgtgaaaagcggcctctctacaggagcaacgcgcttgttcgtctacgcagcatttgataagcctgttattaaaagtggcaaattaacaggtgaaagtagaaacaatgtcacaggatatgtccgattcgatacttctaaagatgaagacaaagttgtcactatgaaaattgcaacatccttaattagtgtcgagcaagcaaagaaaaatctcgaacaggaaatcggattgaatgatacgtttgagggactaaaagaaaaagcgaaaacagagtggaataaaaagttgggcatcatcgaagtagaaggagcatcggaggatcagctcgttacgctttattctaatttataccgcttgttcttgtatccgaattctgcctttgaaaatgtcggaacgacaactgacccagtctataaatatgcaagtccgtattcagcggcaactggccaagatacagcgacaacaactggtgcaaaaatcgtcgacggaaaaacgtatgtcaataacggcttctgggacacttacagaacagcatggcccgcctattctttattaacaccaacatttgctggtgaactgattgatggatttgtccagcaatacagagatggcggctggatagcacggtggtcttcaccaggatttgcaaacttaatgccgggtacgagttcggatgtcgcctttgcggatgcttatcttaaaggggtaacgaattttgatgtccagagcttttaccaatcggcgattcgaaatgcagaggctgtcagtcctaatgccggcacaggccgaaaagggctaacaacttcgatatttgatggatataccaatacttcaacgggtgaaggattagcatgggcaatggatggctatattaacgacttcggtatcgctaatttggcaaaggctctaaaagaaaagggcgacaagagtgatccctactatgctaactatgctgcagattatcagtacttcctgaatcgtgcgcaaaactatgtgcatatgttcaatccatccattgaattttttaatggcagaacagcaaatggggcatggagatcaacaccggataacttcaatcctgccgtttgggggtccgattatacagaaacaaatggctggaatatggcgttccatgtaccacaagacggtcaggggctagccaatctctacggtggaaaagaggggctggcgactaagcttgatcaattctttagcacatcagaaacaggattgtttcctggctcatatggcgggacaatccatgaaatgagagaggcaagagatgtcagaatggggatgtatggccatagcaatcagccgtcccatcatatcgcctacatgtatgattacgctggacagccatggaagacacaggaaaaagtccgtgaagcccttaatcgactttacattggcagtgcaatcggccaaggatattccggggatgaggataacggtgaaatgtccgcctggtatatcttaagcgcaatgggattctatccgcttaaaatgggtacacctgaatatgcgattggtgcaccgttatttaagaaggcaaccatccatttggaaaatggaaaatcaattgttataaacgctcctaacaatagcaaggaaaacaaatatgtgcaaagtatgaaagtaaatggaaaagcctatgctaaaacctccatattacatgcggatatcgccaatggtgccgtaattgattttgaaatgggatccaagccatccaaatggggcagcggagatcaggatattttacaatcgattactccggggtctacagatgggacatcattgtctccactgccattacgtgatgtaacagatcgtttaattgcagcggaaaaaggagctgtaactgtaagtgatgagggaaatggacaattattgtttgataacacatcaaacactcaattatctatgaagagtaaaaccccatcgattgtgtatcaatttaaggaaggcaaacaaaacgtaaaaatgtatacgttgacttcttcaaaagcttcacagaatgaggatccaaaatcttgggtattaaaaggttcaaatgatgggaagagttggagcgtccttgatcaaagaaaaaatgaaacgttccaatggcgtcaatatacgagggcctttacgattcaacatccagggaagtattcacagtataagctcgaaataacagaaaatgctggagcggaggttactactttagcagagcttgagctcctaggctatgatgatgtaaccaatagctatcaggctgtgtatgagctaatggaacaatttaaacagtccaaagacttaacaggaccaatggctgtacaattgaacaacagtttaaccacatcgctagatcatttcaaaaaggatcataaggatcaggcaatcaagcatctggaagatttcttaaagcaccttaataataaaggtttacaggaccggatttcttctaaggcaaaaggggtcctctctgccgatgcgaatcaactgattgtattactagcaagagattaa

SEQ ID NO: 26 - protein, 1411 aa, Bacillus novalis
MKKRAIQKIISSIVSLAVLTSVPVMQKPSVLAASSDSTSASKTDFFSSFEKSDLQLTWTNTVETDANGKKMSSGIDGNVKRDLILGDITDKVVQVTASANNPPNEIDSKLIDGDPTTKWLAFEPTANIVLKLAEPVAVVKYALTSANDAKGRDPKNWTLYGSLDGTNWTAVDTREGEDFKDRFQRNMYDLKNTTKYLYYKLDITKNAGDSITQLAEISLSDGIEVPAPPPGDMKSLIGKGPTSSYTAKTNVGWTGLGALNYSGTHLSDGRAYSYNKLYDVDILVTPATELSYFIAPEFTDKNHNDYSSTYVSVDLAFSDGTYLHDLKAVDQYGVGLNPKDQGDSKYLYVNQWNTIKSTIGSVAAGKTIKRILVAYDNPKGPGAFRGSIDDIKIDGKPVQKAFGSPIDYVNILRGTQSNGSFSRGNNFPAVAIPHGFNFWTPTTNAGSSWIYQYHESNSVNNLPQIQAFSVSHEPSPWMGDRQTFQVMPSASTAATPNANRDSRALEFNHANEIAQPHYYSVKFENGIRTEMTPTDHAAMFKFTFTGATSNLIFDNVNNNGGLTIDAKSGEITGYSDVKSGLSTGATRLFVYAAFDKPVIKSGKLTGESRNNVTGYVRFDTSKDEDKVVTMKIATSLISVEQAKKNLEQEIGLNDTFEGLKEKAKTEWNKKLGIIEVEGASEDQLVTLYSNLYRLFLYPNSAFENVGTTTDPVYKYASPYSAATGQDTATTTGAKIVDGKTYVNNGFWDTYRTAWPAYSLLTPTFAGELIDGFVQQYRDGGWIARWSSPGFANLMPGTSSDVAFADAYLKGVTNFDVQSFYQSAIRNAEAVSPNAGTGRKGLTTSIFDGYTNTSTGEGLAWAMDGYINDFGIANLAKALKEKGDKSDPYYANYAADYQYFLNRAQNYVHMFNPSIEFFNGRTANGAWRSTPDNFNPAVWGSDYTETNGWNMAFHVPQDGQGLANLYGGKEGLATKLDQFFSTSETGLFPGSYGGTIHEMREARDVRMGMYGHSNQPSHHIAYMYDYAGQPWKTQEKVREALNRLYIGSAIGQGYSGDEDNGEMSAWYILSAMGFYPLKMGTPEYAIGAPLFKKATIHLENGKSIVINAPNNSKENKYVQSMKVNGKAYAKTSILHADIANGAVIDFEMGSKPSKWGSGDQDILQSITPGSTDGTSLSPLPLRDVTDRLIAAEKGAVTVSDEGNGQLLFDNTSNTQLSMKSKTPSIVYQFKEGKQNVKMYTLTSSKASQNEDPKSWVLKGSNDGKSWSVLDQRKNETFQWRQYTRAFTIQHPGKYSQYKLEITENAGAEVTTLAELELLGYDDVTNSYQAVYELMEQFKQSKDLTGPMAVQLNNSLTTSLDHFKKDHKDQAIKHLEDFLKHLNNKGLQDRISSKAKGVLSADANQLIVLLARD

SEQ ID NO: 27 - protein, 1378 aa, Bacillus novalis
SSDSTSASKTDFFSSFEKSDLQLTWTNTVETDANGKKMSSGIDGNVKRDLILGDITDKVVQVTASANNPPNEIDSKLIDGDPTTKWLAFEPTANIVLKLAEPVAVVKYALTSANDAKGRDPKNWTLYGSLDGTNWTAVDTREGEDFKDRFQRNMYDLKNTTKYLYYKLDITKNAGDSITQLAEISLSDGIEVPAPPPGDMKSLIGKGPTSSYTAKTNVGWTGLGALNYSGTHLSDGRAYSYNKLYDVDILVTPATELSYFIAPEFTDKNHNDYSSTYVSVDLAFSDGTYLHDLKAVDQYGVGLNPKDQGDSKYLYVNQWNTIKSTIGSVAAGKTIKRILVAYDNPKGPGAFRGSIDDIKIDGKPVQKAFGSPIDYVNILRGTQSNGSFSRGNNFPAVAIPHGFNFWTPTTNAGSSWIYQYHESNSVNNLPQIQAFSVSHEPSPWMGDRQTFQVMPSASTAATPNANRDSRALEFNHANEIAQPHYYSVKFENGIRTEMTPTDHAAMFKFTFTGATSNLIFDNVNNNGGLTIDAKSGEITGYSDVKSGLSTGATRLFVYAAFDKPVIKSGKLTGESRNNVTGYVRFDTSKDEDKVVTMKIATSLISVEQAKKNLEQEIGLNDTFEGLKEKAKTEWNKKLGIIEVEGASEDQLVTLYSNLYRLFLYPNSAFENVGTTTDPVYKYASPYSAATGQDTATTTGAKIVDGKTYVNNGFWDTYRTAWPAYSLLTPTFAGELIDGFVQQYRDGGWIARWSSPGFANLMPGTSSDVAFADAYLKGVTNFDVQSFYQSAIRNAEAVSPNAGTGRKGLTTSIFDGYTNTSTGEGLAWAMDGYINDFGIANLAKALKEKGDKSDPYYANYAADYQYFLNRAQNYVHMFNPSIEFFNGRTANGAWRSTPDNFNPAVWGSDYTETNGWNMAFHVPQDGQGLANLYGGKEGLATKLDQFFSTSETGLFPGSYGGTIHEMREARDVRMGMYGHSNQPSHHIAYMYDYAGQPWKTQEKVREALNRLYIGSAIGQGYSGDEDNGEMSAWYILSAMGFYPLKMGTPEYAIGAPLFKKATIHLENGKSIVINAPNNSKENKYVQSMKVNGKAYAKTSILHADIANGAVIDFEMGSKPSKWGSGDQDILQSITPGSTDGTSLSPLPLRDVTDRLIAAEKGAVTVSDEGNGQLLFDNTSNTQLSMKSKTPSIVYQFKEGKQNVKMYTLTSSKASQNEDPKSWVLKGSNDGKSWSVLDQRKNETFQWRQYTRAFTIQHPGKYSQYKLEITENAGAEVTTLAELELLGYDDVTNSYQAVYELMEQFKQSKDLTGPMAVQLNNSLTTSLDHFKKDHKDQAIKHLEDFLKHLNNKGLQDRISSKAKGVLSADANQLIVLLARD

SEQ ID NO: 28 - DNA, 1095 bp, Chryseobacterium sp.
  sig_peptide 1..66
  mat_peptide 67..1092
  source 1..1095: mol_type = genomic DNA
  CDS 1..1092
atgaaacatttaaagaactttctgttaatactggtatgtatattttctttttcaaaaacctttgctcaaaaggagactgctttaagagataaagttcagatattctattatggatggtatggaaaccaacaaacagatggaagtctgcagcattggaaccatgagatcattccgcattggagcaatccgaaatggaataatcttggacattataaaggagggaatgatattggcgctaatttttatccgggattaggaaattatagttccaatgacaaaaagattataaagaaacacatgcagatgatgaaggattcaggagtgggtgttgtggtgataagctggttgggaaaagattcttttacagataaaagtgtgatgcagtacctggatattgcacaacagtttaatttgaaaatagcatttcacatagaacctttctataaaacaattactgaacttagagatcagctttcttatcttgttgaaaaatattctcagcatccggcattttacaaaaaagacggaaagcctatgtattatgtatatgacagttataaaatcgctccggaagaatggtcaaaactactttccgaaaacggtgaaaaaacagtacgtaatacgaaacttgatgcattgtatatcgggctatgggtagaaaaaaatgattcagagttttttaataagtcaggattcgatggtttttacacttactttgccagtgaaggttttgtgttcgggagtacaacttccaactggaaggatatggctcaatatgcaaaagatcatcatcttatttttattccttgcgtgggtcctgggtattcagatacgaggatcagaccttggaatgaggctaattttaaaagcagagacaatggtaaatattacgaaaaaatgtttgatgctgccactaaagtgaatccggaatttataggaattacttcatttaatgaatggcatgaaggtacgcagatagagcccgcaattcctaagaaaatagacaatttcatctacgaagattacggaaaagatccatggatgtatatcaaagaaacaaaacgcttgacggataagtttctgaaaggaaagtga

SEQ ID NO: 29 - protein, 364 aa, Chryseobacterium sp.
MKHLKNFLLILVCIFSFSKTFAQKETALRDKVQIFYYGWYGNQQTDGSLQHWNHEIIPHWSNPKWNNLGHYKGGNDIGANFYPGLGNYSSNDKKIIKKHMQMMKDSGVGVVVISWLGKDSFTDKSVMQYLDIAQQFNLKIAFHIEPFYKTITELRDQLSYLVEKYSQHPAFYKKDGKPMYYVYDSYKIAPEEWSKLLSENGEKTVRNTKLDALYIGLWVEKNDSEFFNKSGFDGFYTYFASEGFVFGSTTSNWKDMAQYAKDHHLIFIPCVGPGYSDTRIRPWNEANFKSRDNGKYYEKMFDAATKVNPEFIGITSFNEWHEGTQIEPAIPKKIDNFIYEDYGKDPWMYIKETKRLTDKFLKGK

SEQ ID NO: 30 - protein, 342 aa, Chryseobacterium sp.
QKETALRDKVQIFYYGWYGNQQTDGSLQHWNHEIIPHWSNPKWNNLGHYKGGNDIGANFYPGLGNYSSNDKKIIKKHMQMMKDSGVGVVVISWLGKDSFTDKSVMQYLDIAQQFNLKIAFHIEPFYKTITELRDQLSYLVEKYSQHPAFYKKDGKPMYYVYDSYKIAPEEWSKLLSENGEKTVRNTKLDALYIGLWVEKNDSEFFNKSGFDGFYTYFASEGFVFGSTTSNWKDMAQYAKDHHLIFIPCVGPGYSDTRIRPWNEANFKSRDNGKYYEKMFDAATKVNPEFIGITSFNEWHEGTQIEPAIPKKIDNFIYEDYGKDPWMYIKETKRLTDKFLKGK

SEQ ID NO: 31 - DNA, 1267 bp, Aspergillus aculeatus
  sig_peptide 1..54
  mat_peptide 55..1264
  source 1..1267: mol_type = genomic DNA
  CDS 1..483
  CDS 587..1264
atgcacaccacaacactagccctggccctcgccctcgccagcggcacttctgcgacaaactacacggccatgacccgcgccgtgaccgcgctcaacaccctccagacctactacaaccccacgaccgggatctggaacacctgcggctggtggaatggcgccaactgcctaaccacgctggcgaatttgtccctcaagaacagcactgtcaacgacaccgccaccggggtgttcgagaacaccttccgggtggccacgaacacgaacccgtacccggcgcgcgggatcgacgccgactacaccgccgccaacggcaccgcgtacaccatctcgggccagcctacgggggccgccaacgcctcgctgtggctggacgggagctacgacgacgacatgtggtgggggatggcctgggtggccgcgtatgatgtgacgggggtgacggattatctcgatctcgcggagggggtattttaccatcttgtacgttctctatcccccgttcattccactacagggtgtcattcccccttcgatttgagttagggctgtgttgtgtcggaatgcatgctaatggaaacgatagagccgcgcttggccctccctctgcggcaacggcggtctcgactcggactacacgcacgtgtacgtcggcgcgatcagcaacgagctcttcctggcgctgggcgcctcactcgccaaccgcgtcgcgaccaacagcagccgcgagtactacctggactgggcgaagcggcaatgggcgtggtttgagagcagcggcctcatcaacgctaaccacaccatcaacgacgggctgacgggggcgtgcaccaacaacggaatgaccgtgtggtcgtacaaccagggcgtcatcctggggggactggtggagctgcaccgcgccacgggcagccacgcctccaactccacgtacctgactgctgcgggcaagatcgcgcagggggcgatcgccgcgctcgccgacgaggacgacgtcatccatgaatcgtgcgagccggacgcctgtgactcgaacgagacgcagttcaaggggatcttcatccgcaacctcaaggtgttgcagggcgtcgcgccgaatgagacctacgcccgggtgatcaatgcgtcggcggcgagtctgtgggcgaatgatcgcactgacgctacggggttcgggatcgattggtcggggcccgtcgatgcggccacggtcaacgcctcgacgcagagttcggcgctggatgcgctggtcgcggcgatttggtag

SEQ ID NO: 32 - protein, 387 aa, Aspergillus aculeatus
MHTTTLALALALASGTSATNYTAMTRAVTALNTLQTYYNPTTGIWNTCGWWNGANCLTTLANLSLKNSTVNDTATGVFENTFRVATNTNPYPARGIDADYTAANGTAYTISGQPTGAANASLWLDGSYDDDMWWGMAWVAAYDVTGVTDYLDLAEGVFYHLSRAWPSLCGNGGLDSDYTHVYVGAISNELFLALGASLANRVATNSSREYYLDWAKRQWAWFESSGLINANHTINDGLTGACTNNGMTVWSYNQGVILGGLVELHRATGSHASNSTYLTAAGKIAQGAIAALADEDDVIHESCEPDACDSNETQFKGIFIRNLKVLQGVAPNETYARVINASAASLWANDRTDATGFGIDWSGPVDAATVNASTQSSALDALVAAIW

SEQ ID NO: 33 - protein, 369 aa, Aspergillus aculeatus
TNYTAMTRAVTALNTLQTYYNPTTGIWNTCGWWNGANCLTTLANLSLKNSTVNDTATGVFENTFRVATNTNPYPARGIDADYTAANGTAYTISGQPTGAANASLWLDGSYDDDMWWGMAWVAAYDVTGVTDYLDLAEGVFYHLSRAWPSLCGNGGLDSDYTHVYVGAISNELFLALGASLANRVATNSSREYYLDWAKRQWAWFESSGLINANHTINDGLTGACTNNGMTVWSYNQGVILGGLVELHRATGSHASNSTYLTAAGKIAQGAIAALADEDDVIHESCEPDACDSNETQFKGIFIRNLKVLQGVAPNETYARVINASAASLWANDRTDATGFGIDWSGPVDAATVNASTQSSALDALVAAIW

SEQ ID NO: 34 - DNA, 1483 bp, Aspergillus aculeatus
  sig_peptide 1..75
  mat_peptide 76..1480
  source 1..1483: mol_type = genomic DNA
  CDS 1..100
  CDS 159..853
  CDS 905..1480
atggcgagaactaccttccagaacctcctgaaagtggcttatgccgctgcccttgtggcgggcaccgtgtctgccatcgatttggacatcaacaatgaacgtatgatttcaatgaactctactcctcttgtctcctgctgaaatcatcccatctgcagagtccattaaagatgcagccgccaccgccgcgttcaacaccatgcagcactacaacggtaacaagaccggagagattccaggtgtcatccccagcctctggggtgagggcggcgttctcttcaatctcatgatccaatactggtacttcaccggcgacgcctcctacaaccccgccgtcagccagggcatgtactggcagatcggcgacgacgattacatgccgtcgaactggagctcgcagataggcaatgacgaccagatggcctggggccttgccgcgatgacagccgcagagctggactaccctcaggacgttaatcagacatcgtggttgactctggccgaaggcgttttcaacacccaggttgcacggtgggacacgagcaactgcgggggcggactgcgctggcagatctggccctttgagtccggatacacccagaagaatgccatcagcaacggtggtctcttccagctctctgcgcggctggctcggtacacccacaaccagacgtacgcagactgggcggataagatctgggactggagtgccagcgtgccgctcttgaacaacaaaacctggagtatcgccgacagcacgaatgtggacaatggttgcacgacccagggaaacaaccagtggacggccaattacgggccgtacatcagcggtgcagcctatatgtacaactacgtaagtcatttgcaagccagcttagagcaacaacgactaacgagaacgaagaccaacggtcaaaacagcaaatggaagtccggcctcgacggcttgctcaacgtctcgttcgaaaccttcttccccgagaagtacggcggtctgatcctgtcggaaatcctttgtgagcccgcagaggtttgcaactcgctcgaagacacgtacaaggggacttttgtctcggatttggcgctggcaagtctcgtcgctccgtacatctcgtctgaggtgtcttcgcgtctgcaggcgtcggctgtcggagcggcgaagcaatgcacgggtggcaacaatcagacactctgcggtcgtcgctggtattcggacgaatgggacgggactgatgggagagaggaacagctcagtgcaaccagcatctttttcgcgaatatggctggcttcactgggaagggcgttgccactgctgccgcggctgttgatcagacgaccgggtctgccggcacgaatgggacttccacaaacggcactggtgttcctgttgccttgaatggtgctggcagatcgtctcagggacaatttggtgttgccactgctggcgttttagcaggtcttgtattgcttcttgtgctttga

SEQ ID NO: 35 - protein, 457 aa, Aspergillus aculeatus
MARTTFQNLLKVAYAAALVAGTVSAIDLDINNEQSIKDAAATAAFNTMQHYNGNKTGEIPGVIPSLWGEGGVLFNLMIQYWYFTGDASYNPAVSQGMYWQIGDDDYMPSNWSSQIGNDDQMAWGLAAMTAAELDYPQDVNQTSWLTLAEGVFNTQVARWDTSNCGGGLRWQIWPFESGYTQKNAISNGGLFQLSARLARYTHNQTYADWADKIWDWSASVPLLNNKTWSIADSTNVDNGCTTQGNNQWTANYGPYISGAAYMYNYTNGQNSKWKSGLDGLLNVSFETFFPEKYGGLILSEILCEPAEVCNSLEDTYKGTFVSDLALASLVAPYISSEVSSRLQASAVGAAKQCTGGNNQTLCGRRWYSDEWDGTDGREEQLSATSIFFANMAGFTGKGVATAAAAVDQTTGSAGTNGTSTNGTGVPVALNGAGRSSQGQFGVATAGVLAGLVLLLVL

SEQ ID NO: 36 - protein, 432 aa, Aspergillus aculeatus
IDLDINNEQSIKDAAATAAFNTMQHYNGNKTGEIPGVIPSLWGEGGVLFNLMIQYWYFTGDASYNPAVSQGMYWQIGDDDYMPSNWSSQIGNDDQMAWGLAAMTAAELDYPQDVNQTSWLTLAEGVFNTQVARWDTSNCGGGLRWQIWPFESGYTQKNAISNGGLFQLSARLARYTHNQTYADWADKIWDWSASVPLLNNKTWSIADSTNVDNGCTTQGNNQWTANYGPYISGAAYMYNYTNGQNSKWKSGLDGLLNVSFETFFPEKYGGLILSEILCEPAEVCNSLEDTYKGTFVSDLALASLVAPYISSEVSSRLQASAVGAAKQCTGGNNQTLCGRRWYSDEWDGTDGREEQLSATSIFFANMAGFTGKGVATAAAAVDQTTGSAGTNGTSTNGTGVPVALNGAGRSSQGQFGVATAGVLAGLVLLLVL

SEQ ID NO: 37 - DNA, 1352 bp, Aspergillus aculeatus
  sig_peptide 1..72
  mat_peptide 73..1304
  source 1..1352: mol_type = genomic DNA
  CDS 1..97
  CDS 163..857
  CDS 921..1352
atgatcttgtccccgcgctggtggacggctctgctggccgtcctcaccgtcgggcagcacacagtcagagccttacagttagatctcaatgatgagcgtaagcgcatacttcctgttcacgagtatgattcatgcgccaagcactgaccttcttctacatagaatcgatcaagaatgcggccgcgacggcagcctacaacatgatgagctactaccacggcaacgaatctgggcagatcccgggcaaattggtagatacatggtgggaaggaggcgccatgtttatgacgctgattcaatattggtattggaccggcgacacatcctacaacgccgtcaccaccgagggtatgctctggcagaagggccagaacgattatttccccgccaattacagtaactacctcggaaacgatgaccaggtgttctgggggctggccgcaatgacggccgccgagttgaattacccagaggaggacggccaaccgtcttggctgtcgcttgcgcagggggtcttcaacactcaagtaccccgctgggacacgaccagttgtcaaggtggtttgcgctggcagctgtggccctaccaggctggatacaccaccaagaatgcgatctccaacgggggtctcttccaattagcggcgcgactggggcgctacaccaacaatgagacgtacagcaactgggcggagaagatctacgactggatggcgaccaccccgctcctgagggaggaccagtggtccattgctgatacgaccaccacgcagacggagtgtaaagaccatggcgatctccagtggacgtacaactacggaacgtacatcagtggagccgcctatatgtataaccacgtatgacaccgtccttaccataggatctccctcaagtgactgcaggaagctaactagacttagaccaacgggggagacaagtggaagaaggcgttagacggcttgctcggaactaccttgcagaagttcttccctcaagagttcggcggcaacatcatgtccgagatctcctgcgaaccgaacatgatgtgcgatcgcaatcaagactgcttcaaggggtttctgtcatcctggttgaccttcaccaccaccattgcgcctcacaccgcgggcgagatcatccccaaaatccagcaatcggccctggcagcggccaagcaatgctctggcggcaaatcgggcactgaatgcggccgccgctggcaccaggcgacttgggatggggagacttcgctggagtctgatatgagtgcgctgagcgtcttttcttccactatgattgcccacaagggccaagaacagagtcaccaaggaccgctgacttcggagactggg

SEQ ID NO: 38 - protein, 408 aa, Aspergillus aculeatus
MILSPRWWTALLAVLTVGQHTVRALQLDLNDEQSIKNAAATAAYNMMSYYHGNESGQIPGKLVDTWWEGGAMFMTLIQYWYWTGDTSYNAVTTEGMLWQKGQNDYFPANYSNYLGNDDQVFWGLAAMTAAELNYPEEDGQPSWLSLAQGVFNTQVPRWDTTSCQGGLRWQLWPYQAGYTTKNAISNGGLFQLAARLGRYTNNETYSNWAEKIYDWMATTPLLREDQWSIADTTTTQTECKDHGDLQWTYNYGTYISGAAYMYNHTNGGDKWKKALDGLLGTTLQKFFPQEFGGNIMSEISCEPNMMCDRNQDCFKGFLSSWLTFTTTIAPHTAGEIIPKIQQSALAAAKQCSGGKSGTECGRRWHQATWDGETSLESDMSALSVFSSTMIAHKGQEQSHQGPLTSETG

SEQ ID NO: 39 - protein, 384 aa, Aspergillus aculeatus
LQLDLNDEQSIKNAAATAAYNMMSYYHGNESGQIPGKLVDTWWEGGAMFMTLIQYWYWTGDTSYNAVTTEGMLWQKGQNDYFPANYSNYLGNDDQVFWGLAAMTAAELNYPEEDGQPSWLSLAQGVFNTQVPRWDTTSCQGGLRWQLWPYQAGYTTKNAISNGGLFQLAARLGRYTNNETYSNWAEKIYDWMATTPLLREDQWSIADTTTTQTECKDHGDLQWTYNYGTYISGAAYMYNHTNGGDKWKKALDGLLGTTLQKFFPQEFGGNIMSEISCEPNMMCDRNQDCFKGFLSSWLTFTTTIAPHTAGEIIPKIQQSALAAAKQCSGGKSGTECGRRWHQATWDGETSLESDMSALSVFSSTMIAHKGQEQSHQGPLTSETG

SEQ ID NO: 40 - DNA, 1473 bp, Aspergillus aculeatus
  sig_peptide 1..75
  mat_peptide 76..1473
  source 1..1473: mol_type = genomic DNA
  CDS 1..100
  CDS 233..477
  CDS 543..992
  CDS 1054..1473
atgcgcttcctccatggtgctctccatggagggctgcttttaaatgccgctctcttcggccatgttgctcatgccattgatattgatatcagcagtgaatgtatgccttccgctctttgattctatcgcgtcctccagcctgccgtctacctttccataccaatcggggataacttgtggcattctcggataagctgtcacacagaacactgaccatcgtctatcgcaacagcatctataaaggctgcggcaagcaaaacagcatacgggtcaatgacctggtaccacggcaatgagaccggccaaattcctggtgcattccccaccaagtggtgggaaggcagtgcgctgttcatgagtttgcttctctactattattataccggagacagcacctacaacgatgaagttcgccaaggtatgcaatggcaagcaggtgattgcgactacatgccgagcaactacagtagttatctggtatgtaatccggctcgcgtgggatcagactttcggacggaatctcacataaggcatctgggtagggtaacgacgatcaaatgttctggggccttgcggcgatgaccgcggcggagatcgacttcgccgattcgaccgacggatattcctggctcgctcttgctcagggcgtgtacaacacccaagtcgcacgctgggactcgtcgaactgtggaggcggcctgcgctggcagatctggccgtacgaggctggttacgatatgaagaactcaatctcgaacggaggcttgtttcagctggctgcccgtctcgcccgttacacgaacaacgatacgtacgccgactgggccgagaagatcttcgattggtcggcatctgttcctctgttgaataacgaaacgtggaacgttgcagactctaccgacatcgataacggctgcacaacgcagggcaacaaccaatggtcttacaactatggaacatatctgatgggtgccgcatacatgtacaactacgtaaggacgaaacccgactgacctttggttgtgtggacgctgacctgagacaaaatcgcagacgggcaaggccaaatggaagaccgccgtggatggtcttttgaacgtcactctgaccacattcttcccgtccaagtacggcggcaacatcatgtctgaggaactatgtgaacccctggaggtttgcaacgacaacgaaatcctcttcaagggcctgcttagcggatggctcgggtttgtggcgttggtggttccctcgacatatgaccagatcttgccgaagctgcagggctccgccgaggccgctgccgcgtcttgcagcggaatgagcaacaacacgtgtggtgtacggtggtacccgaagtcgtgggatggatggaacggcatggaggaagaaatcgccgtcaccaatgttctttcatctgtcctcattacgacgaagaagtccggtccagtgacctccactaccggcggtaacagt

SEQ ID NO: 41 - protein, 405 aa, Aspergillus aculeatus
MRFLHGALHGGLLLNAALFGHVAHAIDIDISSESSIKAAASKTAYGSMTWYHGNETGQIPGAFPTKWWEGSALFMSLLLYYYYTGDSTYNDEVRQGMQWQAGDCDYMPSNYSSYLGNDDQMFWGLAAMTAAEIDFADSTDGYSWLALAQGVYNTQVARWDSSNCGGGLRWQIWPYEAGYDMKNSISNGGLFQLAARLARYTNNDTYADWAEKIFDWSASVPLLNNETWNVADSTDIDNGCTTQGNNQWSYNYGTYLMGAAYMYNYTGKAKWKTAVDGLLNVTLTTFFPSKYGGNIMSEELCEPLEVCNDNEILFKGLLSGWLGFVALVVPSTYDQILPKLQGSAEAAAASCSGMSNNTCGVRWYPKSWDGWNGMEEEIAVTNVLSSVLITTKKSGPVTSTTGGNS

SEQ ID NO: 42 - protein, 380 aa, Aspergillus aculeatus
IDIDISSESSIKAAASKTAYGSMTWYHGNETGQIPGAFPTKWWEGSALFMSLLLYYYYTGDSTYNDEVRQGMQWQAGDCDYMPSNYSSYLGNDDQMFWGLAAMTAAEIDFADSTDGYSWLALAQGVYNTQVARWDSSNCGGGLRWQIWPYEAGYDMKNSISNGGLFQLAARLARYTNNDTYADWAEKIFDWSASVPLLNNETWNVADSTDIDNGCTTQGNNQWSYNYGTYLMGAAYMYNYTGKAKWKTAVDGLLNVTLTTFFPSKYGGNIMSEELCEPLEVCNDNEILFKGLLSGWLGFVALVVPSTYDQILPKLQGSAEAAAASCSGMSNNTCGVRWYPKSWDGWNGMEEEIAVTNVLSSVLITTKKSGPVTSTTGGNS

SEQ ID NO: 43 - DNA, 1389 bp, Humicola insolens
  sig_peptide 1..69
  mat_peptide 70..1389
  source 1..1389: mol_type = genomic DNA
  CDS 1..100
  CDS 179..900
  CDS 967..1389
atgctccccaagccagcggcgctccttcgcgccatgctgcttgcggccatcgccgccgatgcccagtccgacaagctccaagtcgacctcaactctccaggtgcgtctttgacacggtagttcgcctccgttgcttcgaggataattctcgctgacccaagcgcctctgcatctccagcctcgatcaagaaagccgcaaaactggtcgctgccaacctcatgtcccactaccatggcgacgaacccggtgctactcctggtatcctcccaggcccgcctcctgctggtccatactactggtggcaagccggtgctatgtgggggaccatagtcgactactggcactacacgggagacgaaacatacaacgccgaagcgctgcggtccatggtcttccaggctgaacctcccgccaacgcctacatgccccgcaactggaccgcgtccctaggtaacgacgatcaaggtttctggggtatggccgccatgctcgccgccgagacgaacttcaccaacccaccagaagaccagcctcaatggctagcccttgcccaggccgtcttcaacacccaggtcccgcgttgggagatggactactgcaacggtggtctgcgatggcaaatcgtgcaggccaacaacggctacaactacaagaacaccatcgctgctgccgtcttcctcaacattgcttcccgtctggcccgatacaccggcaacgactcgtacgcggaatgggctgaaagggcttgggactggatggagggtgtcggatacatcaccgaggacttcaatgttaaggatggtgcccacgtcgaatccaactgcaccgacatcaaccccgtccagttctctgccaacgctgccatcctgatccacggcgtttccgtcatgtacaactacgtatgttcactacctgcttgacttgctccattatctcccgacctattccttgctaacccaactcagacatccgggagcgcccgcgataaatggcgttaccgcgtcgttggccttgtcaaccgcaccctcgaacactttttccccgacggcatcatggtcgaacgcccctgcgaactcgaagaccgcatgcaatgcaacacagaccagcactccttcaaagggtacatgcaccgcgccctcgccacagccgccgtcgtcgcccccttcatgcgcgataccatcgtccccgtcctccgctcttccaccgaaggctgcgtctcctcctgcctcgccgacggaacctgcggtttccgttggaacattggccgctacgacggcgacgtcgaccacggtcccgccggtcagcagatgagcgccctcgccgcgctgtcgaccttgctgatcgatcaggatcgggttctgcgcgggccgttgacgaacgcgaccggc

SEQ ID NO: 44 - protein, 415 aa, Humicola insolens
MLPKPAALLRAMLLAAIAADAQSDKLQVDLNSPASIKKAAKLVAANLMSHYHGDEPGATPGILPGPPPAGPYYWWQAGAMWGTIVDYWHYTGDETYNAEALRSMVFQAEPPANAYMPRNWTASLGNDDQGFWGMAAMLAAETNFTNPPEDQPQWLALAQAVFNTQVPRWEMDYCNGGLRWQIVQANNGYNYKNTIAAAVFLNIASRLARYTGNDSYAEWAERAWDWMEGVGYITEDFNVKDGAHVESNCTDINPVQFSANAAILIHGVSVMYNYTSGSARDKWRYRVVGLVNRTLEHFFPDGIMVERPCELEDRMQCNTDQHSFKGYMHRALATAAVVAPFMRDTIVPVLRSSTEGCVSSCLADGTCGFRWNIGRYDGDVDHGPAGQQMSALAALSTLLIDQDRVLRGPLTNATG

SEQ ID NO: 45 - protein, 392 aa, Humicola insolens
DKLQVDLNSPASIKKAAKLVAANLMSHYHGDEPGATPGILPGPPPAGPYYWWQAGAMWGTIVDYWHYTGDETYNAEALRSMVFQAEPPANAYMPRNWTASLGNDDQGFWGMAAMLAAETNFTNPPEDQPQWLALAQAVFNTQVPRWEMDYCNGGLRWQIVQANNGYNYKNTIAAAVFLNIASRLARYTGNDSYAEWAERAWDWMEGVGYITEDFNVKDGAHVESNCTDINPVQFSANAAILIHGVSVMYNYTSGSARDKWRYRVVGLVNRTLEHFFPDGIMVERPCELEDRMQCNTDQHSFKGYMHRALATAAVVAPFMRDTIVPVLRSSTEGCVSSCLADGTCGFRWNIGRYDGDVDHGPAGQQMSALAALSTLLIDQDRVLRGPLTNATG

SEQ ID NO: 46 - DNA, 1336 bp, Humicola insolens
  sig_peptide 1..90
  mat_peptide 91..1336
  source 1..1336: mol_type = genomic DNA
  CDS 1..118
  CDS 180..1336
atgtcgccatcagccagacaacggggcccggcgcgatggctggctgccgccctcgcgacaagcagtgtgctgttttcgacgacacgggcgcaacagtattataagatcgacacaataggtagcgaagctgatgccctcgtatgctttgcgcggtgggctcgagactaacaggtgaccagaggagatcaaggagtcggcgcgcactcttgcatacgacttgatgctctattacaagggcaaccagtctggcgaaatccccggtatcttgccaggtcccccgaccgaacacaaaggcgactactactggtgggaaggcggcgcgatgatgggtacatacgtcgactactggtttttgacccgcgacccaagctacaaccacgtcgtcatggaaggcatgcttcaccaagtcggtcccaacgctgattacatgccacccaaccataccgcttctctcggtaacgacgaccaaggattctggggcatgtcggctatgctcgccgccgagaacaagtttcccaacccacccgaagaccagccacagtggttggcccttgcccaggccgtcttcaacacgcaggctgccccggagcggcacgacggcacctgcaacggcggtctccgttggcaggttccccccacgaatgccgggtacaactacaaaaacaccatcgccaacgcctgtttcttcgacctcggagcccgcctggcccgttacaccaagaacgagacgtacgccaattgggccaacaacattttcgattggttgatgggcgtcggatacatcgacaccagggaacccggctggcgagtttatgacggcgcccatgtcgagcataactgcaccgatatcaacaaagcccagttcagctacaacgctgcccttctcttgcacggcgcggccttcatgtacaactacaccaacggcgaggagaaatggaagacgcgcatcgatggcctgatcgagggtatcttgcgcgactttttcaaggacggcgctgcctatgagttgccgtgcgagggtcgtcagggtgcgtgtaccacggacatgttgagcttcaagggttacatgcaccgctggatggcggtcgtgaccaaggtggctccttatacggcggaaaagatcctccctgcgcttcgcacgtccaccgaggccgcggtggctcaatgcacgggtccgccgacgggccgtcgttgcggcttctactggtccacccgtcaatatgtcgataccgccgtcgacaagaccagcggcgccggagaggccatgaatgtccttgctgcagtctcgagcttgctcattgagtacgcggatccaccagcgaccaacgagacgggc

SEQ ID NO: 47 - protein, 425 aa, Humicola insolens
MSPSARQRGPARWLAAALATSSVLFSTTRAQQYYKIDTIEEIKESARTLAYDLMLYYKGNQSGEIPGILPGPPTEHKGDYYWWEGGAMMGTYVDYWFLTRDPSYNHVVMEGMLHQVGPNADYMPPNHTASLGNDDQGFWGMSAMLAAENKFPNPPEDQPQWLALAQAVFNTQAAPERHDGTCNGGLRWQVPPTNAGYNYKNTIANACFFDLGARLARYTKNETYANWANNIFDWLMGVGYIDTREPGWRVYDGAHVEHNCTDINKAQFSYNAALLLHGAAFMYNYTNGEEKWKTRIDGLIEGILRDFFKDGAAYELPCEGRQGACTTDMLSFKGYMHRWMAVVTKVAPYTAEKILPALRTSTEAAVAQCTGPPTGRRCGFYWSTRQYVDTAVDKTSGAGEAMNVLAAVSSLLIEYADPPATNETG

SEQ ID NO: 48 - protein, 395 aa, Humicola insolens
QQYYKIDTIEEIKESARTLAYDLMLYYKGNQSGEIPGILPGPPTEHKGDYYWWEGGAMMGTYVDYWFLTRDPSYNHVVMEGMLHQVGPNADYMPPNHTASLGNDDQGFWGMSAMLAAENKFPNPPEDQPQWLALAQAVFNTQAAPERHDGTCNGGLRWQVPPTNAGYNYKNTIANACFFDLGARLARYTKNETYANWANNIFDWLMGVGYIDTREPGWRVYDGAHVEHNCTDINKAQFSYNAALLLHGAAFMYNYTNGEEKWKTRIDGLIEGILRDFFKDGAAYELPCEGRQGACTTDMLSFKGYMHRWMAVVTKVAPYTAEKILPALRTSTEAAVAQCTGPPTGRRCGFYWSTRQYVDTAVDKTSGAGEAMNVLAAVSSLLIEYADPPATNETG

SEQ ID NO: 49 - DNA, 1422 bp, Humicola insolens
  sig_peptide 1..63
  mat_peptide 64..1422
  source 1..1422: mol_type = genomic DNA
  CDS 1..85
  CDS 161..640
  CDS 696..937
  CDS 1015..1422
atgtacaccacaagctccgtagtagcggcggccgccctcctcggggccggcgctgcgaatgccgcctacagcatcgacactgtcggtacgtatatttcctacctcagttctttgatgcgccgccttctaagtacctagatatctaactccgctttcccagccgacataaaaaagaccgccgccaccgtcgcctgggacctgatgcagtactaccacggcaacgagacgggccaaacccccggcatcctccccggcccccctcccgcgggggactactactggtgggaggccggcgccatgtgggggacgctgatcgactactggaagtacacgggcgacgactcgtacaacgccgtcatcacccaggccatggtgcaccaggccggccccaaccgcgactacatgcctcccaacgtgaccctctctctgggcaacgacgaccaaggcttctggggcatgtcggccatgctggccgccgagctccgcttccccgatccgccgccggaccagccccagtggctggctttggcgcaggccgtgtttaatacgcaggcgagcccagaccgccatgacgagacgtgcaacggcgggttgaggtggcagatcccgtggtcgaatcccggttatgactacaagaacagtgagttggtctgtatcggaaacaggaccgagacagagttgctgacggggaccagctatcgccaacggctgcttcttcaacctcggcgcccgtctggcccgctacaccagaaacaagacgtatgccgagtgggctgagaagacgtgggactgggtcgagggcgtcgggtatatcaccaaggactaccaagtgtatgacggcgcccatgtggaccataactgcaccgacctcaaccgggcccagttctcctacaacaacgccatctttctcctcggcgcggccttcatgtacaactatgtaagcccgtccatttcccccggttcctgcgcgcatttacctcctctgacttgctaacctccacttcctccacccagaccgacggcagccccaaatggcgcgaccgcgtcgaaggcctcgtcgacggcgccatccgcgatttcttcccggacggcgtcgccttcgaggtgccctgtgagaccaacatgacttgcacgaccgacatgctcagcttcaaggggtacctgcaccgctggctggcggcggccaccacggtggcgcccttcatcgcgcccaaggtgctgcccgtgctgcgcagctcggccgaggcggccatctcgacctgcacgggcgaggccgacggccgcacgtgcggcttccagtgggccaagcgccagtatgacggcagcaagggcgccgggcagcagatgaacgtcctgggcgccgtgtcggcgctgatggtggagcataatccggattatgtgatggtgacggcggactcgggc

SEQ ID NO: 50 - protein, 405 aa, Humicola insolens
MYTTSSVVAAAALLGAGAANAAYSIDTVADIKKTAATVAWDLMQYYHGNETGQTPGILPGPPPAGDYYWWEAGAMWGTLIDYWKYTGDDSYNAVITQAMVHQAGPNRDYMPPNVTLSLGNDDQGFWGMSAMLAAELRFPDPPPDQPQWLALAQAVFNTQASPDRHDETCNGGLRWQIPWSNPGYDYKNTIANGCFFNLGARLARYTRNKTYAEWAEKTWDWVEGVGYITKDYQVYDGAHVDHNCTDLNRAQFSYNNAIFLLGAAFMYNYTDGSPKWRDRVEGLVDGAIRDFFPDGVAFEVPCETNMTCTTDMLSFKGYLHRWLAAATTVAPFIAPKVLPVLRSSAEAAISTCTGEADGRTCGFQWAKRQYDGSKGAGQQMNVLGAVSALMVEHNPDYVMVTADSG

SEQ ID NO: 51 - protein, 384 aa, Humicola insolens
AYSIDTVADIKKTAATVAWDLMQYYHGNETGQTPGILPGPPPAGDYYWWEAGAMWGTLIDYWKYTGDDSYNAVITQAMVHQAGPNRDYMPPNVTLSLGNDDQGFWGMSAMLAAELRFPDPPPDQPQWLALAQAVFNTQASPDRHDETCNGGLRWQIPWSNPGYDYKNTIANGCFFNLGARLARYTRNKTYAEWAEKTWDWVEGVGYITKDYQVYDGAHVDHNCTDLNRAQFSYNNAIFLLGAAFMYNYTDGSPKWRDRVEGLVDGAIRDFFPDGVAFEVPCETNMTCTTDMLSFKGYLHRWLAAATTVAPFIAPKVLPVLRSSAEAAISTCTGEADGRTCGFQWAKRQYDGSKGAGQQMNVLGAVSALMVEHNPDYVMVTADSG

SEQ ID NO: 52 - DNA, 1337 bp, Humicola insolens
  sig_peptide 1..60
  mat_peptide 61..1337
  source 1..1337: mol_type = genomic DNA
  CDS 1..85
  CDS 154..1337
atgaggttactaccttggttgacggcgtctcttggtgtgctggtgaggtcggcggcggctatcgagttggatttggacaatgaaggtgaggaggcacttgtccaaggactgtctttgatattcatgtctttcagaagctgacacccccctcagaatccatcaaggccgccgcgagcaccatcgccttcggcctggtccgctactacaccggcaactacaccggcgacactcccggcaacctgccggacccctacttctggtgggaggccggcgccatgttcggcaccctcgtcgactactgggctctgaccggcgacgagtcctacaacgccatcaccctccaagccatggtccatcagggcaccgagaagggcgacttcatgccgcggaaccagacccggaccctcggcaacgacgaccaaggcttctggggcatggcggccatgtcggccgccgagaacaacttccccaacccgccgcacgaccagccccaatggctcgccctcgcccagtccctcttcaaccagtgggcgtcccgctgggagcccgaaacctgcggcggcggcctgcgctggcaaatctttgccttcaacaacggcttcaactacaaaaactccatctccaacggctgtttcttcaacatcgccgcccgcctggcccgctacacgggcaaccagacctacgccgactgggccgcccgcatctgggactgggaggagggcatcgggctgatcacgcccgactacgccgtgcacgacggcgtcggcatcaaccctctgaacggcgagtgcctggtcggcagcatggacacgaaccagtggacctacaacgccggcatcttcctccacggcgcggccgtcatgtacaacctcaccaacggcagcgccgactggcgcgcgcgcgtcgacggcatcctctccaacaccatcaacaccttttacaccaccccggaacccggcagccccgccggcgccaaccccgtcctgagggagctctgcgaagggccccaaaacttctgcaacatcgaccagcgctccttcaaaggctacctcacgcgctggctggccggcacctccctgctcgccccgcacacccagcccgtcatccagcccctcctgcgctcctccgccctcgccgccgcccacgcttgctccggcccgacccagcccccggaattcaaaggccacacgggaaccgcctgcggtctccgctggaccaccgccgccgggttcgacggcctcgtcggcgtcggcgagcagatgaatgccttgtcggccgtcatgtacacgctcgccgcgcgcccgggcgcgccggagccgttgaccgcggatacgggc

SEQ ID NO: 53 - protein, 423 aa, Humicola insolens
MRLLPWLTASLGVLVRSAAAIELDLDNEESIKAAASTIAFGLVRYYTGNYTGDTPGNLPDPYFWWEAGAMFGTLVDYWALTGDESYNAITLQAMVHQGTEKGDFMPRNQTRTLGNDDQGFWGMAAMSAAENNFPNPPHDQPQWLALAQSLFNQWASRWEPETCGGGLRWQIFAFNNGFNYKNSISNGCFFNIAARLARYTGNQTYADWAARIWDWEEGIGLITPDYAVHDGVGINPLNGECLVGSMDTNQWTYNAGIFLHGAAVMYNLTNGSADWRARVDGILSNTINTFYTTPEPGSPAGANPVLRELCEGPQNFCNIDQRSFKGYLTRWLAGTSLLAPHTQPVIQPLLRSSALAAAHACSGPTQPPEFKGHTGTACGLRWTTAAGFDGLVGVGEQMNALSAVMYTLAARPGAPEPLTADTG

SEQ ID NO: 54 - protein, 403 aa, Humicola insolens
IELDLDNEESIKAAASTIAFGLVRYYTGNYTGDTPGNLPDPYFWWEAGAMFGTLVDYWALTGDESYNAITLQAMVHQGTEKGDFMPRNQTRTLGNDDQGFWGMAAMSAAENNFPNPPHDQPQWLALAQSLFNQWASRWEPETCGGGLRWQIFAFNNGFNYKNSISNGCFFNIAARLARYTGNQTYADWAARIWDWEEGIGLITPDYAVHDGVGINPLNGECLVGSMDTNQWTYNAGIFLHGAAVMYNLTNGSADWRARVDGILSNTINTFYTTPEPGSPAGANPVLRELCEGPQNFCNIDQRSFKGYLTRWLAGTSLLAPHTQPVIQPLLRSSALAAAHACSGPTQPPEFKGHTGTACGLRWTTAAGFDGLVGVGEQMNALSAVMYTLAARPGAPEPLTADTG

SEQ ID NO: 55 - DNA, 1605 bp, synthetic construct
  misc_feature 1..1605: note = Synthetic Construct
  sig_peptide 1..105
  mat_peptide 106..1605
  source 1..1605: mol_type = other DNA
  CDS 1..1605
atggccagtaaggttagaagaagattcaagcttatcgtattgcttgttgttcttgcactctcgatcagtaccctatctccaagcggaatgcctcgcgcccatgcttttgaagcggaggatgcgaagacggcaatcgttgcctacaatgatgcattctgggatgcaaacgcgaaatacttctggaaatccaccaatcgtaccgattatcaggacttttggatagaggccgagctttgggagctcgttatggatgcctatctgcatacatcggacccggagctcaaggcgcagctgcggacccagattgacgatgtgttcgatggagccgtaacgaggtacggcgaggactggacatataatccgtataacgatgacatcatgtggtgggcaatggcaagtgcaagagcctatcagattacgaatgacgaacggtatttggaacaagcggaatattatttcaattatgtctatgataatgaatgggatactgaatttgccggaggcggcatttggtggaaaagcgatgatcgcacgacaaaaaatgcatgcatcaactttcctgccgctcaaaccgctgtctttttgtataacgttacccaagacgaacaatacctggatgcagccgaaaccatctaccattggggaaaaacaatactgactgacggcaacggcaaagtattcgatcgaattgaaacgcaaaatggagctattcaaggcgcgactcactataatcaaggtgcgtttattgggtcagccgcaggtctctacgagattacgggagacacggattatctggacgatgcgatcaaagcggccacgtatacgaaggagcatatggttgacgtcaacggactgctgcgatatgagggccctaacggagatttgaagggcggcaaaacgattctgctgcgcaacctcggctattttcaagcggcgatagacgctcgccaggaagaaaattaccaatccttcgccgaaagctacaatgagtggctggcttttaatgccgatatggcatggaacaatcgcaatgcggccaatctcgtcgacggcaactgggcgggacaacagttatccggcgccatcgagtcatggagcgcggcagcggccgtacaagctttgatatccctaaagccgcagaacgcggtacagctgggatatgccgttaagaacccttataacaggatcgaagcggaaagttacaatattataaacggccctggcctggaggacagcaatgaaggctcgcaacaactggccggtattcaggacagtcattatgcggcatataaaaatgtcgattttggttcggaagatggtgcaagcggtttcattgcgcgagcatcgagcggaacgggcggcggccagatcgaaatcagactggatgctttggacggaccaaaggcaggtacgctaaatgtaaatggtaccggaggctggaacaactacatcgacgcggctgtactgctgaaggatgagcaagggaatccgagtcctgtgaccggcgtacacgatgtgtatctggtgtttaagaggacaaatgacacatatctattcaatctcaattggttccaatttacgaaggtcgatccgaccctgatttct

SEQ ID NO: 56 - protein, 535 aa, synthetic construct
  REGION 1..535: note = Synthetic Construct
MASKVRRRFKLIVLLVVLALSISTLSPSGMPRAHAFEAEDAKTAIVAYNDAFWDANAKYFWKSTNRTDYQDFWIEAELWELVMDAYLHTSDPELKAQLRTQIDDVFDGAVTRYGEDWTYNPYNDDIMWWAMASARAYQITNDERYLEQAEYYFNYVYDNEWDTEFAGGGIWWKSDDRTTKNACINFPAAQTAVFLYNVTQDEQYLDAAETIYHWGKTILTDGNGKVFDRIETQNGAIQGATHYNQGAFIGSAAGLYEITGDTDYLDDAIKAATYTKEHMVDVNGLLRYEGPNGDLKGGKTILLRNLGYFQAAIDARQEENYQSFAESYNEWLAFNADMAWNNRNAANLVDGNWAGQQLSGAIESWSAAAAVQALISLKPQNAVQLGYAVKNPYNRIEAESYNIINGPGLEDSNEGSQQLAGIQDSHYAAYKNVDFGSEDGASGFIARASSGTGGGQIEIRLDALDGPKAGTLNVNGTGGWNNYIDAAVLLKDEQGNPSPVTGVHDVYLVFKRTNDTYLFNLNWFQFTKVDPTLIS

SEQ ID NO: 57 - protein, 500 aa, synthetic construct
  REGION 1..500: note = Synthetic Construct
FEAEDAKTAIVAYNDAFWDANAKYFWKSTNRTDYQDFWIEAELWELVMDAYLHTSDPELKAQLRTQIDDVFDGAVTRYGEDWTYNPYNDDIMWWAMASARAYQITNDERYLEQAEYYFNYVYDNEWDTEFAGGGIWWKSDDRTTKNACINFPAAQTAVFLYNVTQDEQYLDAAETIYHWGKTILTDGNGKVFDRIETQNGAIQGATHYNQGAFIGSAAGLYEITGDTDYLDDAIKAATYTKEHMVDVNGLLRYEGPNGDLKGGKTILLRNLGYFQAAIDARQEENYQSFAESYNEWLAFNADMAWNNRNAANLVDGNWAGQQLSGAIESWSAAAAVQALISLKPQNAVQLGYAVKNPYNRIEAESYNIINGPGLEDSNEGSQQLAGIQDSHYAAYKNVDFGSEDGASGFIARASSGTGGGQIEIRLDALDGPKAGTLNVNGTGGWNNYIDAAVLLKDEQGNPSPVTGVHDVYLVFKRTNDTYLFNLNWFQFTKVDPTLIS

SEQ ID NO: 58 - protein, 27 aa, synthetic construct
  REGION 1..27: note = Synthetic construct
MKKPLGKIVASTALLISVAFSSSIASA

SEQ ID NO: 59 - protein, 8 aa, synthetic construct
  REGION 1..8: note = Synthetic construct
HHHHHHPR

SEQ ID NO: 60-64 - skipped (5 entries)

SEQ ID NO: 65 - protein, 9 aa, synthetic construct
  REGION 1..9: note = Synthetic construct
KNTPANAPA

SEQ ID NO: 66 - skipped

SEQ ID NO: 67 - protein, 299 aa, Bacillus bogoriensis
ANSGFYVSGTTLYDANGNPFVMRGINHGHAWYKDQATTAIEGIANTGANTVRIVLSDGGQWTKDDIHTVRNLISLAEDNHLVAVLEVHDATGYDSIASLNRAVDYWIEMRSALIGKEDTVIINIANEWFGSWEGDAWADGYKQAIPRLRNAGLNHTLMVDAAGWGQFPQSIHDYGREVFNADPQRNTMFSIHMYEYAGGNASQVRTNIDRVLNQDLALVIGEFGHRHTNGDVDEATIMSYSEQRGVGWLAWSWKGNGPEWEYLDLSNDWAGNNLTAWGNTIVNGPYGLRETSRLSTVFQ

SEQ ID NO: 68 - protein, 297 aa, Paenibacillus species
MATGFYVSGNKLYDSTGKPFVMRGVNHGHSWFKNDLNTAIPAIAKTGANTVRIVLSNGSLYTKDDLNAVKNIINVVNQNKMIAVLEVHDATGKDDYNSLDAAVNYWISIKEALIGKEDRVIVNIANEWYGTWNGSAWADGYKKAIPKLRNAGIKNTLIVDAAGWGQFPQSIVDYGQSVFAADSQKNTVFSIHMYEYAGKDAATVKANMENVLNKGLALIIGEFGGYHTNGDVDEYAIMRYGQEKGVGWLAWSWYGNSSGLNYLDMATGPNGSLTSFGNTVVNDTYGIKNTSQKAGIF

SEQ ID NO: 69 - protein, 464 aa, Bacillus hemicellulosilyticus
QTHSGFYIEGSTLYDANGEPFVMRGINHGHAWYKHDSNVAIPAIANQGANTIRIVLSDGGQWAKDDINTLNQVLDLAEEHEMIAVVEVHDATGSNSMADLNRAVDYWIEMKDALIGKEDRVIINIANEWYGAWDGQGWANGYKEVIPRLRNAGFTHTLMVDAAGWGQYPQSIHDYGQEVFNADPLANTMFSIHMYEYAGGNASMVQSNIDGVVDQGLALVIGEFGHMHTDGDVDEATILSYSQQRGVGWLAWSWKGNGTQWEYLDLSYDWQGTNLTSWGNTIVHGPNGLLETSIPSSIFHTAPNNGDPPPHNGNETILYDFEHGTQGWSGSSLLGGPWTTNEWSTNGNHSLKADIFLSANSKHELAKVENRNLSGYSTLQATVRHAHWGNVGNLTARMYVKTGSNYSWFNGDPIPVNSANGTTVTLPLSSIPNLNDVKEIGVEFIGASNSNGQTAIYLDHVTIQ